SEQUENCE LISTING (117 sequences)

SEQ ID NO: 1 - protein, 572 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGAPVEIRRVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGGMGGGMF

SEQ ID NO: 2 - protein, 9 aa, Homo sapiens
QMRPVSRVL

SEQ ID NO: 3 - protein, 9 aa, Homo sapiens
VMAPRTVLL

SEQ ID NO: 4 - protein, 9 aa, Homo sapiens
VMAPRTLVL

SEQ ID NO: 5 - protein, 9 aa, Homo sapiens
VMAPRTLLL

SEQ ID NO: 6 - protein, 9 aa, Homo sapiens
IMAPRTLVL

SEQ ID NO: 7 - protein, 9 aa, Homo sapiens
VMAPRTLIL

SEQ ID NO: 8 - protein, 9 aa, Homo sapiens
VMAPRTLFL

SEQ ID NO: 9 - protein, 9 aa, Homo sapiens
LMLQGVDLL

SEQ ID NO: 10 - protein, 9 aa, Homo sapiens
VMLAVDAVI

SEQ ID NO: 11 - protein, 9 aa, Homo sapiens
GMKFDRGYI

SEQ ID NO: 12 - protein, 9 aa, Homo sapiens
KMKISHKRL

SEQ ID NO: 13 - protein, 9 aa, Homo sapiens
QMRPVSRVL

SEQ ID NO: 14 - protein, 9 aa, Homo sapiens
LMLQGVDLL

SEQ ID NO: 15 - protein, 9 aa, Homo sapiens
VMLAVDAVI

SEQ ID NO: 16 - protein, 9 aa, Homo sapiens
GMQFDRGYL

SEQ ID NO: 17 - protein, 9 aa, Homo sapiens
AMTKDNNLL

SEQ ID NO: 18 - protein, 9 aa, Homo sapiens
TMKPVQKVL

SEQ ID NO: 19 - protein, 9 aa, Homo sapiens
PMGRGTKVI

SEQ ID NO: 20 - protein, 9 aa, Homo sapiens
IMDNCEELI

SEQ ID NO: 21 - protein, 9 aa, Homo sapiens
EMLQQSKIL

SEQ ID NO: 22 - protein, 9 aa, Homo sapiens
RMKENQKHI

SEQ ID NO: 23 - protein, 9 aa, Homo sapiens
RMIKLGLGI

SEQ ID NO: 24 - protein, 9 aa, Homo sapiens
MMKLIINSL

SEQ ID NO: 25 - protein, 9 aa, Homo sapiens
RMKEKQDKI

SEQ ID NO: 26 - protein, 9 aa, Homo sapiens
RMLRLSLNI

SEQ ID NO: 27 - protein, 20 aa, Homo sapiens
MLRLPTVFRQMRPVSRVLAP

SEQ ID NO: 28 - protein, 14 aa, Homo sapiens
MLLPTVFQMRPVSR

SEQ ID NO: 29 - protein, 20 aa, Homo sapiens
MLPLPTVFHQMRPVSRLLAP

SEQ ID NO: 30 - protein, 9 aa, Homo sapiens
AMGPRGLLL

SEQ ID NO: 31 - protein, 9 aa, Homo sapiens
QMSPALTCL

SEQ ID NO: 32 - protein, 9 aa, Homo sapiens
GMAPALRHL

SEQ ID NO: 33 - protein, 9 aa, Homo sapiens
KMWPVLWTL

SEQ ID NO: 34 - protein, 9 aa, Homo sapiens
EMLPTAILL

SEQ ID NO: 35 - protein, 9 aa, Homo sapiens
QMLPLLWLL

SEQ ID NO: 36 - protein, 9 aa, Homo sapiens
LMPPPLLLL

SEQ ID NO: 37 - protein, 9 aa, Homo sapiens
AMAPRSRLL

SEQ ID NO: 38 - protein, 9 aa, Homo sapiens
TMMPRLPTL

SEQ ID NO: 39 - protein, 9 aa, Homo sapiens
KMTPRSRIL

SEQ ID NO: 40 - protein, 9 aa, Homo sapiens
TMDPQLRLL

SEQ ID NO: 41 - protein, 9 aa, Homo sapiens
GMTPSKGVL

SEQ ID NO: 42 - protein, 9 aa, Homo sapiens
PMIPLYRNL

SEQ ID NO: 43 - protein, 9 aa, Homo sapiens
IMVPLARIL

SEQ ID NO: 44 - protein, 9 aa, Homo sapiens
DMAPLRSKL

SEQ ID NO: 45 - protein, 9 aa, Homo sapiens
PMNPNTNDL

SEQ ID NO: 46 - protein, 27 aa, Homo sapiens
QMGPQEENLEMCPDIPVLYMEPALRCL

SEQ ID NO: 47 - protein, 9 aa, Homo sapiens
GMGPDYRVL

SEQ ID NO: 48 - protein, 9 aa, Homo sapiens
FMKPGKVVL

SEQ ID NO: 49 - protein, 9 aa, Homo sapiens
RMGPGIDRL

SEQ ID NO: 50 - protein, 9 aa, Homo sapiens
FMPPGLQEL

SEQ ID NO: 51 - protein, 9 aa, Homo sapiens
QMRPLFLEL

SEQ ID NO: 52 - protein, 9 aa, Homo sapiens
VMDPKKDVL

SEQ ID NO: 53 - protein, 9 aa, Homo sapiens
MMGPYRQDL

SEQ ID NO: 54 - protein, 9 aa, Homo sapiens
RMFPNAPYL

SEQ ID NO: 55 - protein, 9 aa, Homo sapiens
GMPPGIPPL

SEQ ID NO: 56 - protein, 9 aa, Homo sapiens
IMNPSYYNL

SEQ ID NO: 57 - protein, 9 aa, Homo sapiens
QMKPRQLII

SEQ ID NO: 58 - protein, 9 aa, Homo sapiens
QMKPVKSVL

SEQ ID NO: 59 - protein, 9 aa, Homo sapiens
SMGPVGSPL

SEQ ID NO: 60 - protein, 9 aa, Homo sapiens
GMDPVARRL

SEQ ID NO: 61 - protein, 9 aa, Homo sapiens
TMPPVGTDL

SEQ ID NO: 62 - protein, 9 aa, Homo sapiens
LMTPVAALL

SEQ ID NO: 63 - protein, 9 aa, Homo sapiens
RMRPVKAAL

SEQ ID NO: 64 - protein, 9 aa, Homo sapiens
RMRPVKKAL

SEQ ID NO: 65 - protein, 9 aa, Homo sapiens
SMEPVVEQL

SEQ ID NO: 66 - protein, 9 aa, Homo sapiens
LMTPVQPML

SEQ ID NO: 67 - protein, 9 aa, Homo sapiens
AMNPVIYTL

SEQ ID NO: 68 - protein, 9 aa, Homo sapiens
EMTPVTIDL

SEQ ID NO: 69 - protein, 9 aa, Homo sapiens
EMPPVQFKL

SEQ ID NO: 70 - protein, 9 aa, Homo sapiens
RMAPVCESL

SEQ ID NO: 71 - protein, 9 aa, Homo sapiens
SMHPVTAML

SEQ ID NO: 72 - protein, 9 aa, Homo sapiens
KMGPVLCHL

SEQ ID NO: 73 - protein, 9 aa, Homo sapiens
EMQPVVFVL

SEQ ID NO: 74 - protein, 9 aa, Homo sapiens
HMNPVSNGL

SEQ ID NO: 75 - protein, 9 aa, Homo sapiens
LMDPVKQML

SEQ ID NO: 76 - protein, 9 aa, Homo sapiens
SMEPVVRRL

SEQ ID NO: 77 - protein, 9 aa, Homo sapiens
LMHPVLESL

SEQ ID NO: 78 - protein, 9 aa, Homo sapiens
TMIPVLLNL

SEQ ID NO: 79 - protein, 9 aa, Homo sapiens
SMVPVQVQL

SEQ ID NO: 80 - protein, 9 aa, Homo sapiens
CMEPVDAAL

SEQ ID NO: 81 - protein, 9 aa, Homo sapiens
FMVPVACGL

SEQ ID NO: 82 - protein, 9 aa, Homo sapiens
VMAPVDVDL

SEQ ID NO: 83 - protein, 9 aa, Homo sapiens
VMIPVFDIL

SEQ ID NO: 84 - protein, 9 aa, Homo sapiens
QMAPVQKNL

SEQ ID NO: 85 - protein, 9 aa, Homo sapiens
LMRPVQKEL

SEQ ID NO: 86 - protein, 9 aa, Homo sapiens
EMAPWFALL

SEQ ID NO: 87 - protein, 9 aa, Homo sapiens
LMLPDFDLL

SEQ ID NO: 88 - protein, 9 aa, Homo sapiens
TMDPDHRLL

SEQ ID NO: 89 - protein, 9 aa, Homo sapiens
VMDPDHRLL

SEQ ID NO: 90 - protein, 9 aa, Homo sapiens
LMEPPAVLL

SEQ ID NO: 91 - protein, 9 aa, Homo sapiens
NMVPSSRLL

SEQ ID NO: 92 - protein, 9 aa, Homo sapiens
WMGPISPLL

SEQ ID NO: 93 - protein, 9 aa, Homo sapiens
AMSPWLLLL

SEQ ID NO: 94 - protein, 9 aa, Homo sapiens
DMKPENVLL

SEQ ID NO: 95 - protein, 9 aa, Homo sapiens
RMEPIRWLL

SEQ ID NO: 96 - protein, 9 aa, Homo sapiens
AMGPYVFLL

SEQ ID NO: 97 - protein, 9 aa, Homo sapiens
LMLPDFDLL

SEQ ID NO: 98 - protein, 9 aa, Homo sapiens
LMQPNFELL

SEQ ID NO: 99 - protein, 9 aa, Homo sapiens
RMTPALLLL

SEQ ID NO: 100 - protein, 9 aa, Homo sapiens
EMGPYPALL

SEQ ID NO: 101 - protein, 9 aa, Homo sapiens
HMGPSRNLL

SEQ ID NO: 102 - protein, 9 aa, Homo sapiens
LMRPLWLLL

SEQ ID NO: 103 - protein, 9 aa, Homo sapiens
VMLPKAALL

SEQ ID NO: 104 - protein, 9 aa, Homo sapiens
KMLPEASLL

SEQ ID NO: 105 - protein, 9 aa, Homo sapiens
EMLPAFDLL

SEQ ID NO: 106 - protein, 9 aa, Homo sapiens
WMQPSVVLL

SEQ ID NO: 107 - protein, 9 aa, Homo sapiens
TMDPIRDLL

SEQ ID NO: 108 - protein, 9 aa, Homo sapiens
FMDPLKLLL

SEQ ID NO: 109 - protein, 9 aa, Homo sapiens
PMAPPTALL

SEQ ID NO: 110 - protein, 9 aa, Homo sapiens
AMCPIAMLL

SEQ ID NO: 111 - DNA, 94 bp, Homo sapiens
cggaattcatgcttcggttacccacagtctttcgccagatgagaccggtgtccagggtactggctcctcatctcactcgggcttatggatccgc

SEQ ID NO: 112 - DNA, 31 bp, Homo sapiens
cagtctttcgccaggggagaccggtgtccag

SEQ ID NO: 113 - protein, 10 aa, Homo sapiens
RGPGRAFVTI

SEQ ID NO: 114 - protein, 9 aa, Homo sapiens
VMAPVTVLL

SEQ ID NO: 115 - protein, 9 aa, Homo sapiens
AMAPRTLLL

SEQ ID NO: 116 - protein, 9 aa, Homo sapiens
QMRPVSRAL

SEQ ID NO: 117 - protein, 9 aa, Homo sapiens
AMAPVTLLL

(19) United States
(12) Patent Application Publication
(10) Pub. No.: US 2007/0081991 A1
(43) Pub. Date: Apr. 12, 2007

(54) Compositions and methods for modulation of immune responses

(75) Inventor: Karl Petter Soderstrom, San Francisco, CA (US)

Correspondence Address: PERKINS COIE LLP, P.O. BOX 2168, MENLO PARK, CA, 94026, US

(73) Assignee: Pepgen Corporation

(21) Appl. No.: 11/522,051

(22) Filed: Sep. 15, 2006

Related U.S. Application Data
(63) Continuation of application No. 10/210,148, filed on Jul. 31, 2002, now abandoned.
(60) Provisional application No. 60/308,598, filed on Jul. 31, 2001.

(51) Int. Cl. A61K 39/395; A61K 38/10; C07K 7/08; C40B 40/10; C40B 30/06

(52) U.S. Cl. 424/133.1; 514/15; 530/328; 435/7.1

(57) ABSTRACT

The present invention relates to a novel mechanism for modulation of immune response. More closely, the present invention relates to modulation of CD94/NKG2 receptor yfunction by HLA-E+bound peptides causing either inhibition or absence of inhibition of said receptors. In a preferred embodiment the invention relates to HLA-E binding hsp (heat shock protein) 60 peptides.

### CROSS-REFERENCE TO RELATIVE APPLICATION

This application is a continuation of application Ser. No. 10/210,148, filed Jul. 31, 2002, which claims the benefit of U.S. Provisional Application No. 60/308,598, filed Jul. 31, 2001, each of which is hereby incorporated herein by reference in its entirety.

### TECHNICAL FIELD

The present invention relates to novel compositions and methods for modulating immune responses in mammalian subjects. More specifically, the invention relates to modulation of CD94/NKG2 receptor function by HLA-E+ binding peptides causing either inhibition or absence of inhibition of said receptors.

### SEQUENCE LISTING

The instant application contains a sequence listing which has been submitted in a computer readable form (CRF), along with a paper copy as part of the specification, and is hereby incorporated by reference in its entirety. The CRF is identical to the paper copy of the sequence listings.

### BACKGROUND OF THE INVENTION

Natural killer (NK) cells are lymphocytes involved in the innate immune response against certain microbial and parasitic infections. Recent reports also suggest important roles for NK cells in experimental autoimmune models, but still little is known about the function of NK cells during autoimmune disease in man. In this paper we have studied the expression of killer cell immunoglobulin (Ig)-like (KIR) and C-type lectin-like (CD94/NKG2) receptors specific for MHC class I molecules on NK cells, as well as on ab T cells and gd T cells derived from synovial fluid (SF) and peripheral blood (PB) of patients with arthritis, mainly rheumatoid arthritis (RA). We found that the SF of arthritic patients contained an increased proportion of NK cells as compared to paired PB. In contrast to PB-NK cells, the SF-NK cell population almost uniformly expressed the CD94/NKG2A cell surface receptor and contained drastically reduced proportions of KIR+ NK cells. Functional analysis revealed that both in vitro cultured polyclonal SF-NK cells and PB-NK cells from patients are fully capable of killing a range of target cells. SF-NK cell cytolysis was, however, inhibitied by the presence of HLA-E on transfected target cells. When blocking CD94 on the SF-NK cells or by masking HLA on autologous cells, the SF-NK cells were capable to-perform self-directed lysis. Thus, HLA-E may play a fundamental role in the regulation of a major NK cell population in the inflamed joint.

MHC class I molecules regulate natural killer (NK) cell functions such as their capability to mediate lysis of target cells (Ljunggren et al., Immunol. Today 11: 237-244, 1990, incorporated herein by reference). This regulation is controlled by a complex repertoire of MHC class I specific receptors displayed on the NK cell surface. These receptors monitor expression of MHC class I on neighboring cells and deliver an inhibitory signal that blocks NK cell-mediated cytotoxicity of MHC class I-expressing, normal cells (Lanier et al., Immunity 6:371-378, 1997, incorporated herein by reference).

HLA-E is a widely distributed, non-classical MHC class I molecule expressed on the cell surface in association with beta 2-microglobulin. HLA-E is widely expressed in association with b2-microglobulin and peptide on the surface of cells, albeit at low levels (Wei et al., Hum. Immunol. 29:131, 1990, incorporated herein by reference). The peptide loading of HLA-E is believed to be TAP-dependent, although there are reports of TAP-independent presentation. In contrast to classical MHC class I molecules, HLA-E displays a rather limited polymorphism, and its peptide binding cleft is primarily occupied by nonameric peptides derived from the signal sequence of certain HLA-A, -B, -C, and -G molecules (Lazetic et al., J. Immunol. 157:4741-4745, 1996, incorporated herein by reference). These peptides generally share a common motif: methionine at position 2, and leucine or isoleucine at position 9 (Arnett et al., Arthritis Rheum. 31:315-324, 1988, incorporated herein by reference). Analysis of the crystal structure of HLA-E has clarified the peptide selectivity of this molecule (Söderström et al., J. Immunol. 159:1072-1075, 1997, incorporated herein by reference). The murine homologue of HLA-E, designated Qa-1b, also primarily presents peptides derived from the signal sequence of some mouse MHC class I molecules, with similarly conserved anchor residues at positions 2 and 9 (Miller et al. Proc. Natl. Acad. Sci. USA. 70:190-194, 1973; Hendrich et al., Arthritis Rheum. 34:423-431, 1991, incorporated herein by reference). It has, however, recently been demonstrated that both HLA-E and Qa-1b can bind a diverse array of peptides derived from random peptide libraries (Fort et al., J. Immunol. 161: 3256-3261, 1998; Phillips et al., Immunity 5:163-72, 1996, each incorporated herein by reference). In addition, it has been reported that Qa-1b can present peptides derived from a mouse and bacterial heat shock protein 60 (hsp60), and that these complexes can be detected by T cells via their antigen-specific T cell receptor (TCR) (Litwin et al., J. Exp. Med. 180:537-543, 1994, incorporated herein by reference).

The conserved anchor motif found within signal sequences of some MHC class I molecules is thought to be important for binding to pockets in the HLA-E peptide binding cleft. The HLA-E molecule, when loaded with these HLA class I signal peptides, it thought to form a functional ligand for C-type lectin like receptor dimers designated CD94/INKG2A, -B, -C, -E, which are expressed on NK cells and subsets of T cells.

At least two distinct types of inhibitory receptors have been described in man, the killer cell immunoglobulin (Ig)-like receptors (KIR) and the C-type lectin-like receptors. There are several distinct KIRs that are characterized by either two (2D) or three (3D) extracellular Ig-like domains, with either short (S) or long (L) cytoplasmic tails. Based on their structure, KIRs are subgrouped in families, and certain members having three Ig-domains (KIR3DL) specifically recognize groups of HLA-B molecules, whereas other KIRs with two Ig-domains (KIR2DL) recognize subgroups of HLA-C molecules. In addition, a homodimer of two KIR3DL molecules is reported to recognize HLA-A molecules (Long et al., http://www.ncbi.nlm.nih.gov/prow/guide/679664748 g.htm, 1999, incorporated herein by reference). The C-type lectin-like receptors that comprise CD94 covalently associated with members in the NKG2 family (NKG2A, -B, and -C) (Chang et al., Eur. J. Immunol. 25:2433-2437, 1995; Lazetic et al., J. Immunol. 157:47414745, 1996, incorporated herein by reference) specifically recognize the relatively non-polymorphic HLA-E molecule (Braud et al., Nature 391:795-799, 1991, incorporated herein by reference). The CD94/NKG2A receptor is believed to mediate an inhibitory signal to NK cells upon recognition by the cells of HLA-E loaded with proper peptides expressed on bystander target cells. This CD94/NKG2A mediated signal is thought to prevent NK cell activation (e.g. cytotoxicity and cytokine release) during encounter with normal autologous cells. NK cells bearing CD94/NKG2A receptors that regulate their self-tolerance are capable of killing cells that have lost the expression of protective HLA-E molecules. Protective HLA-E molecules are those that are loaded with peptides derived from the signal sequence of certain other MHC class I molecules.

Earlier, it was appreciated that a hybrid construct consisting of a HLA-G leader sequence grafted onto HLA-B*5801 transfected into 721.221 cells significantly upregulated protective endogenous HLA-E levels in this cell line (Braud et al., 1991 supra). These experiments suggested that an HLA class I leader must be present for stable mature HLA-E protein to form and migrate to the cell surface to be detected by CD94/NKG2A inhibitory receptors.

CD94/NKG2 receptors are expressed by a large proportion of NK cells, both in human and mouse, and interact with the non-classical MHC class I molecule HLA-E and its murine homologue Qa-1b, respectively Vance et al., J. Exp. Med. 188:1841, 1998; Braud et al., Nature 391:6669:795, 1998, each incorporated herein by reference). NKG2A contains an intracellular immunoreceptor tyrosine-based inhibitory motif (ITIM) mediating inhibitory signals (Brooks et al., J. Exp. Med. 185:795, 1997, incorporated herein by reference), whereas NKG2C associates with the immunoreceptor tyrosine-based activating motif (ITAM) bearing adaptor molecule DAP-12, and mediates positive signaling (Lanier et al., Immunity 8:693, 1998, incorporated herein by reference). CD94/NKG2A/C receptors have been reported to discriminate between different HLA-E and Qa-1b binding peptides (Kraft et al., J. Exp. Med. 192:613, 2000; Llano et al., Eur. J. Immunol. 28:2854, 1998; Vales-Gomez et al., Embo J. 18:4250, 1999; Brooks et al., J. Immunol. 162: 305, 1999, each inch), but the physiological significance of this selectivity remains unclear.

In order to avoid autoimmune attack mediated by NK cells, it has been proposed that at least one MHC class I-specific inhibitory receptor for one self-MHC class I molecule should be expressed by each single NK cell (Lanier et al., Immunity 6:371-378, 1997, incorporated herein by reference). Since most normal cells usually express sufficient levels of all MHC class I molecules they are therefore protected from NK cell-mediated attack. However, the loss or down-regulation of one or several MHC class I molecule(s), which is common during certain viral infections and neoplastic transformation, may render such cells susceptible to destruction by NK cells (Id.) Also, lymphocytes from patients with autoimmune disease, including rheumatoid arthritis (RA), show a defective expression of MHC class I (Fu et al., J. Clin. Invest. 91:2301-2307, 1993, incorporated herein by reference). It is unknown if this has an impact on the NK cell tolerance, and the role for NK cells, in general, in RA remains still obscure.

Recent studies, however, in various experimental models of autoimmune diseases have pointed to a regulatory role for NK cells that appears to be of pathological significance. For example, NK cells seem to play an important role in down-regulating TH1-mediated colitis by controlling the responses of effector T cells in a perforin dependent manner (Fort et al., J. Immunol. 161:3256-3261, 1998, incorporated herein by reference). In experimental autoimmune encephalomyelitis (EAE), a model for human multiple sclerosis (MS), administration of the NK cell stimulatory compound linomide can protect mice from developing disease, and in the same model depletion of NK cells led to increased production of THI cytokines and an exacerbation of disease (Matsumoto et al., Eur. J. Immunol. 28:1681-1688, 1998; Zhang et al., J. Exp. Med. 186:1677-1687, 1997, each incorporated herein by reference). These reports suggest that the presence of NK cells is beneficial for the protection against prototype TH1-mediated diseases. In contrast, a pathogenic role for NK cells was suggested in a murine model of asthma, a prototype TH2-mediated disease, where depletion of NK cells protected mice from developing allergen-induced inflammation in the airway epithelium (Korsgren et al., J. Exp. Med. 189:553-562, 1999, incorporated herein by reference).

RA is an autoimmune disease characterized by chronic inflammation of joints leading to progressive destruction of cartilage and bone. After the onset of RA, the synovial compartment contains not only activated T cells but also granzyme-positive NK cells (Tak et al., Arthritis Rheum. 37:1735-1743, 1994, incorporated herein by reference). Although potent NK cell stimulating cytokines such as IL-15 can be found within the joint (Thurkow et al., J. Pathol. 181:444-450, 1997, incorporated herein by reference), freshly isolated synovial NK cells appear less cytotoxic, and less prone to produce IFN-γ, as compared to NK cells derived from peripheral blood (PB) (Lipsky Clin. Exp. Rheumatol. 4:303-305, 1986; Berg et al., Clin. Exn. Immunol. 1:174-182, 1999, each incorporated herein by reference). Since signalling through KIR- as well as CD94/NKG2 molecules is known to regulate both NK cell-mediated cytotoxicity and cytokine production, it is of importance to investigate the receptor usage for NK cells at inflammatory sites.

Heat shock proteins (hsps), exemplified by hsp60, are highly conserved through evolution between man and bacteria. Hsp60 is present in all living cellular organisms (Lindquist et al., Annu. Rev. Genet. 22:631, 1988; Bukau et al., Cell 92:351, 1998, each incorporated herein by reference). In eukaryotic cells it serves a vital function as a mitochondrial chaperone, and in bacteria as an intracellular protein involved in the assembly and disassembly of multi-subunit protein complexes (Fink, Physiol. Rev. 79:425, 1999, incorporated herein by reference). Increased levels of hsp60 are induced in response to a variety of stress stimuli, e.g. temperature increase, nutrient deprivation, exposure to toxic chemicals, inflammatory responses and allograft rejection (Lindquist, 1988, supra; Anderton et al., Eur. J. Immunol. 23:33, 1993; Birk et al., Proc. Natl. Acad. Sci. USA 96:5159, 1999, each incorporated herein by reference). Hsp60 is believed to play an important role in the protection of cells from the consequences of these harmful stimuli. At the same time it may render these cells more susceptible to attack by hsp60-directed innate and adaptive immune responses, and it is known that hsp60 is highly immunogenic. For example, an immune response elicited against bacterial-hsp60 during an infection may cross-react with self-hsp60.

Hsp60 is the dominant self antigen in mammalian autoimmunity. The fact that endogenous hsp60 expression is highly elevated in chronically inflamed tissues (such as, for example, in the rheumatoid joint) has generated considerable interest among research groups studying autoimmune mechanisms and disesease. Increased levels of hsp60 is also found during cellular stress, e.g. during hyperthermia. Whole-body hyperthermia is used as a therapy against cancer.

Based on these and other reports, there does not appear to be a clear teaching or suggestion in the art to use a common agent for modulating HLA-E/CD94/NKG2 cellular receptor interactions, or for controlling aberrant immune responses associated with changes in HLA-E/CD94/NKG2 cellular receptor interactions and/or with cellular stress factors and associated disease states, including inflammation and autoimmunity. Similarly, the role of stress-induced proteins and peptides that may be associated with modulation of HLA-E/CD94/NKG2 cellular receptor interactions and aberrant regulation of immune responses attending such conditions as chronic inflammation and autoimmunity is yet to be elucidated.

In view of the foregoing, there remains an urgent need in the art for additional tools and methods to modulate HLA-E/CD94/NKG2 cellular receptor interactions, and to control aberrant immune responses, particularly those associated with changes in HLA-E/CD94/NKG2 cellular receptor interactions potentially mediated by cellular stress factors. A related need exists for effective compositions and methods to alleviate symptoms of associated disease states, including inflammation, autoimmunity, and cancer. Surprisingly, the instant invention fulfills these objects and satisfies additional objects and advantages that will become apparent from the following description.

### SUMMARY OF THE INVENTION

The present invention provides methods and compositions that employ a proinflammatory or anti-inflammatory binding peptide to modulate an immune response in a mammalian subject. Typically, the binding peptide binds a major histocompatibility complex class I (MHC class I) molecule, for example a HLA-E MHC class I molecule, on an antigen presenting cell (APC) and the bound complex of the proinflammatory or anti-inflammatory binding peptide and HLA-E interacts with a MHC class I-specific inhibitory receptor. The MHC class I-specific inhibitory receptor will typically be a CD94/NKG2 cellular receptor. The interactions between the proinflammatory or anti-inflammatory binding peptide and HLA-E binding peptide modulates interactions between the binding peptide/HLA-E complex and the receptor to yield novel regulation of an immune response in a population of cells expressing the inhibitory receptor.

In more detailed aspects of the invention, the interactions between the proinflammatory or anti-inflammatory binding peptide and HLA-E binding peptide either facilitates a proinflammatory or anti-inflammatory response in a cell population or other subject, e.g., a mammalian subject with an autoimmune disease, inflammatory disease or condition (e.g., chronic inflammation, or inflammation attending surgery or trauma), graft rejection, viral infection, cancer, or other disease or condition amenable to treatment by modulating an immune reponse according to the invention.

In certain embodiments of the invention, an anti-inflammatory binding peptide interacts with an HLA-E molecule on the surface of a cell presenting the peptide bound to the HLA-E, and the resulting peptide-HLA-E complex is recognized by the MHC class I-specific inhibitory receptor. This recognition leads to a protective immune response, characterized by decreased cytotoxic activity and/or induction of expression of one or anti-inflammatory cytokine(s) by the cell bearing a CD94/NKG2 cellular receptor.

In additional aspects, the proinflammatory or anti-inflammatory binding peptide of the invention may exhibit activity of upregulating expression of HLA-E molecules on cells exposed to the peptide, in vitro or in vivo.

In other embodiments of the invention, a proinflammatory binding peptide binds with an HLA-E molecule on the surface of a cell that presents the peptide bound to the HLA-E, and the resulting peptide-HLA-E complex interferes with protective recognition by the MHC class I-specific inhibitory receptor. That is, the binding of the peptide inhibits a protective immune response mediated by the CD94/NKG2 cellular receptor. This inhibition of CD94/NKG2 receptor-mediated protection involves competion for binding HLA-E between the proinflammatory binding peptide and one or more protective (i.e., anti-inflammatory) peptides that are rendered ineffective or impaired by binding competition with the proinflammatory binding peptide. In this context, the proinflammatory binding peptide competitively occupies the HLA-E binding cleft, and the complex between the proinflammatory binding peptide and HLA-E is not recognized by the CD94/NKG2 cellular receptor. The inhibition of CD94/NKG2 cellular receptor-mediated protection is reflected by increased cytotoxic activity and/or induction of expression of one or more proinflammatory cytokine(s) by a cell bearing the CD94/NKG2 cellular receptor (e.g., a NK or T cell).

In the case of a proinflammatory binding peptide, the peptide will have biological activity if it competes with an anti-inflammatory binding peptide for binding to the MHC class I molecule, and/or stimulates a cytotoxic or proinflammatory cytokine induction response in cells expressing the CD94/NKG2 cellular receptor.

The phrase “antigen presenting cells” refers to a class of cells capable of presenting antigen to cells of the immune system that are capable of recognizing antigen when it is associated with a major histocompatibility complex molecule. Antigen presenting cells generally mediate an immune response to a specific antigen by processing the antigen into a form that is capable of associating with a major histocompatibility complex molecule on the surface of the antigen presenting cell. Antigen presenting cells include such diverse cell types as macrophages, T cells and synthetic (“artificial”) cells.

Typically, the immune response subject to modulation by the methods and compositions of the invention include cytotoxic responses and induction of proinflammatory and anti-inflammatory cytokines in cells expressing the MHC class I-specific inhibitory receptor. In exemplary embodiments, these cells are selected from natural killer (NK) cells and cytotoxic T lymphocytes (CTLs). The immune response induced may be suppression or enhancement of one or more activities of NK or T cells, including suppression or enhancement of cytotoxic activity, cytokine production, proliferation, chemotaxis, etc.

The methods of the invention generally comprise exposing a subject to an effective amount of a proinflammatory or anti-inflammatory binding peptide of the invention that will bind to HLA-E molecules on a surface of the subject and elevate or inhibit the binding of CD94/NKG2 cellular receptor to the peptide/HLA-E complex at the surface. Within certain methods of the invention, the subject is an isolated or bound CD94/NKG2 cellular receptor, a membrane or cell preparation comprising the receptor, a cell population, tissue or organ expressing the receptor, or a mammalian patient. In more detailed embodiments, the subject comprises a cell population, tissue or organ selected for in vivo or ex vivo treatment or diagnostic processing. Alternatively, the subject may be a mammalian patient susceptible to an inflammatory or autoimmune disesease or condition, viral infection, graft rejection or cancer. The proinflammatory or anti-inflammatory binding peptide may in these cases be administered in a prophylactic or therapeutic effective dose to prevent or inhibit a related disease condition or symptom.

In additional detailed embodiments of the invention, the proinflammatory or anti-inflammatory binding peptide is administered to the subject in an amount effective to elevate or inhibit one or more biological activities selected from (a) binding by a CD94/NKG2 cellular receptor to a cell surface, an HLA-E molecule, or an HLA-E/peptide complex (b) cytotoxic or cytokine induction activity of a APC (e.g., NK cell or CTL), or (c) a disease symptom or condition associated with an inflammatory or autoimmune disorder, viral infection, graft rejection, or cancer.

The proinflammatory or anti-inflammatory binding peptide may be naturally occurring or synthetic. Often, the peptide is a peptide analog or mimetic, or an allelic variant found among native proinflammatory or anti-inflammatory binding peptide sequences. The peptide, peptide analog or mimetic can be modified in a wide variety of ways, e.g., by addition, admixture, or conjugation of additional amino acids, peptides, proteins, chemical reagents or moieties which do not substantially alter the biological activity (e.g., HLA-E binding activity) of the peptide.

In additional aspects, the invention relates to an assay for HLA-E binding peptides or analogues, comprising the steps: a) providing a peptide library; b) forming HLA-E/peptide complexes; c) selecting stable complexes capable of inhibiting or activating CD94/NKG2 receptors on NK and T cells; and d) isolating of a stable peptide/peptide analogue from said complex.

In other aspects, the invention relates to a pharmaceutical composition comprising any of the peptides according to the invention in a pharmaceutically acceptable carrier. Within the methods and compositions of the invention, the proinflammatory or anti-inflammatory binding peptide may be formulated in various combinations with a pharmaceutically acceptable carrier, diluent, excipient, adjuvant or other active or inactive agents, in an amount or dosage form sufficient to prevent or alleviate one or more selected disease conditions or symptoms identified herein below.

In yet additional aspects of the invention, the proinflammatory or anti-inflammatory binding peptide is administered according to the foregoing methods in a combinatorial formulation or coordinate treatment protocol with one or more additional anti-viral, anti-inflammatory, anti-cancer, or anti-graft rejection therapeutic active agent(s). Within related methods and compositions, the proinflammatory or anti-inflammatory binding peptide is admixed or coadministered (simultaneously or sequentially) with one or more of these adjunct therapeutic agents to prevent or alleviate one or more selected disease conditions or symptoms identified herein below.

The instant invention also includes kits, packages and multicontainer units containing a proinflammatory or anti-inflammatory binding peptide, optionally with other active or inactive ingredients, and/or means for administering the same for use in the diagnosis, management and/or prevention and treatment of a selected disease condition or symptom identified herein below. Typically, these kits include a diagnostic or pharmaceutical preparation of the proinflammatory or anti-inflammatory binding peptide, typically formulated with a biologically suitable carrier and optionally contained in a bulk dispensing container or unit or multi-unit dosage form. Optional packaging materials may include a label or instruction which indicate a desired use of the kit as described herein below.

Additional aspects of the invention include polynucleotide molecules and vector constructs encoding proinflammatory or anti-inflammatory binding peptides of the invention, including peptide mimetics and analogs.

Also provided are vaccines and other immunogenic compositions that elicit an immune response involving production of antibodies targeting one or more proinflammatory or anti-inflammatory binding peptides of the invention, which may be useful for diagnostic and/or therapeutic purposes as described in further detail below. Also provided within the invention are a variety of additional diagnostic and therapeutic tools and reagents as set forth in detail in the following description.

### BRIEF DESCRIPTION OF THE DRAWINGS

FIG. 1 provides the protein sequence of human hsp60. The mitochondrial targeting signal is shown in gray. Boxed are the four peptide sequences displaying a methionine followed by leucine or isoleucine seven amino acids C-terminally, two important residues for binding to HLA-E pockets. Hsp60sp corresponds to residues 10-18 in the sequence (QMRPVSRVL) (SEQ ID NO: 2).

FIG. 2 depicts stabilization of HLA-E by hsp60sp and B7sp on K562 cells transfected either with HLA-E*0101 or HLA-E*01033. Cell surface expression of HLA-E*0101 (upper panel) and HLA-E*01033 (lower panel) after overnight incubation at 26° C. with 300 mM of either hsp60sp (left panel, bold line) or B7sp (right panels bold line). The dashed line represents HLA-E expression after incubation with 300 mM of a control peptide (P18I10). Cells were stained with anti-MHC class I mAb DX17, followed by RPE-conjugated goat-anti-mouse IgG. The HLA-E expression was confirmed by staining with the anti-HLA-E mAb 3D12. Staining with isotype matched control antibody is shown as shaded gray. One representative experiment out of more than 10 is shown.

FIG. 3 documents upregulation of HLA-E by overexpression of the full-length hsp60 signal peptide is enhanced by cellular stress. HLA-E surface expression was monitored on cells growing at increasing densities. Cells were collected and analyzed for HLA-E expression between day 1 and day 5 (as indicated on the top of the histograms). The numbers in the top right corner of each histogram indicate cell density (cells/ml) and percent viability at the time of analysis, respectively. The numbers in the lower right corner of each histogram in (a) indicate the MFI of HLA-E expression (top, black) and the MFI of GFP (bottom, gray). The numbers in the lower right corner of each histogram in (b) indicate the MFI of HLA-E expression. All cells were stained with an HLA specific antibody (DX17, dashed line) or with control Ig (gray histogram), followed by RPE-conjugated goat anti-mouse IgG. (a) K562 cells co-transfected with HLA-E*01033 and the full-length (residues 1-26) wild type hsp60 signal peptide-GFP construct (wild-type hsp60L, top panel) or mutant hsp60 signal peptide-GFP (mutated hsp60L, lower panel), cultured at increasing cell density. A gate was set on GFP positive cells and 10 000 events were acquired within this gate. (b) K562 cells (upper panel) and K562 transfected with HLA-E*01033 (K562 E*01033, lower panel) cultured at increasing cell density. Note that the K562-E*01033 cell line in (b) and the co-transfected cell lines presented in figure (a) were generated and selected independently, which may account for the higher HLA-E background level observed at dayl. Therefore the absolute levels of HLA-E should not be directly compared between FIGS. 3a and 3b.

FIG. 4 shows binding of soluble HLA-E tetrameric molecules to CD94/NKG2 receptors. (a) Ba/F3 cells transfected with CD94 and NKG2A were incubated with HLA-E/B7sp tetramers-(bold line), HLA-E/hsp60sp-tetramers (thin line), or control H-2Db/gp33-tetramers (dashed line). (b) Ba/F3 cells transfected with CD94, NKG2C and DAP-12 were incubated with HLA-E/B7sp-tetramers (bold line), HLA-E/hsp60sp-tetramers (thin line), or control H-2Db/gp33-tetramers (dashed line). (c) The NK cell line NKL was incubated with HLA-E/B7sp-tetramers (bold line), HLA-E/hsp60sp-tetramers (thin line), or control H-2Db/gp33-tetramers (dashed line). (d) HB-120 B-cell hybridoma (anti-MHC class I) was incubated with HLA-E/B7sp-tetramers (bold line), HLA-E/hsp60sp-tetramers (thin line), or control H-2Db/gp33-tetramers (dashed line). All incubations were done at 4° C. for 45 min in PBS supplemented with 1% FCS. HLA-E/hsp60sp-tetramers failed to bind both CD94/NKG2A+ and CD94/NKG2C+ cells over a range of HLA-E/hsp60sp-tetramer concentrations. This is one representative experiment of more than 5 that were conducted.

FIG. 5 shows that hsp60sp fails to protect K562-E*01033 cells from killing by NK cells. K562-E*01033 cells were incubated with the different peptides at 26° C. for 15-20 hours, and then tested in 2 h 51Cr release assays. In order to ensure that the levels of HLA-E with a protective peptide, and not the HLA-E levels as such, provided the protective capacity we kept the non-protective peptides, but omitted the B7sp, during the assays. (a) Killing of K562-E*01033 cells by NKL (left) or Nishi (right) after incubation with 300 mM P18I10 control peptide, 300 mM hsp60sp, or 30 mM B7sp. 50 mM of P18I10 control peptide and hsp60sp was also included during the assays. Data from an E:T ratio of 30:1 is shown. The figure represents the mean of at least three experiments. Error bars indicate standard error of the mean. (b) Killing of K562-E*01033 cells by NKL (left panel) or Nishi (right panel) incubated over night with 30 mM 7sp, 300 mM P18I10 (pCtrl), 300 mM B7 R5V, 300 mM hsp60sp, or 300 mM hsp60 V5R. 50 mM of all peptides, except B7sp, were included during the assay. Peptide concentrations were chosen according to FIG. 5c. The figure represents the mean of at least three experiments. Error bars indicate standard error of the mean. (c) HLA-E cell surface expression by K562-E*01033 after the assay. A cold target preparation was prepared in parallel as in (a) and (b), and then stained with DX17 mAb (anti-HLA class I), followed by RPE-conjugated goat-anti-mouse IgG. One representative example out of more than 5 is shown. Note that, as in (a) and (b), 50 mM of all peptides, except for B7sp, was present during the time of the assay, explaining the lower HLA-E expression with B7sp compared to Hsp60sp, Hsp60 V5R and B7 R5V. (d) Killing of K562-E*01033 cells by Nishi after incubation for 30 min at room temperature with 0.1 mM B7sp and increasing amounts of competing peptides (hsp60sp, hsp60.4, B7 R5V and P18I10). All the peptides were kept throughout the assay

FIG. 6 demonstrates increased HLA-E cell surface levels on K562-E*01033 after cellular stress does not protect from NK cell mediated killing. (a) Killing of K562-E*01033 cells (grown at increasing cell densities as in FIG. 3b) by NKL in a 2 h 51Cr release assay. (b) Same experimental setting as above, in the presence of 100 mM B7sp. Closed circles-high density; open squares-medium density; closed triangles-low density. (c) HLA-E expression on the K562-E*01033 cells after culture at increasing cell density.

FIG. 7 demonstrates an increased proportion of NK cells present in the synovial fluid (SF) of patients with rheumatoid arthritis (RA). Freshly isolated mononuclear cells from the SF and PB of RA patients and PB of healthy controls were stained with mAbs against CD56 (PE-conjugated) and CD3 (Cychrome-conjugated). A gate was set on the lymphocyte population and approximately 10,000 events were aquired and analyzed by flow cytometry. The results are shown as mean±SEM of the percentage of CD56⁺CD3⁻ cells within the lymphocyte gate. There was an increased proportion of CD56⁺CD3⁻ NK cells among lymphocytes in the SF (14.1±2.2%) as compared to patient PB (9.4±1.3%; p<0.05).

FIGS. 8A-8C demonstrates that SF-NK cells are CD94^{bright} and NKG2A⁺, phenotypically resembling a minor subset of CD56^{bright} PB-NK cells. FIG. 8A: Freshly isolated mononuclear cells from PB (upper histograms) and SF derived from the right and left knee (middle and lower histogram rows, respectively) of a representative RA patient, were triple-stained with antibodies against CD94 (DX22; thick line, middle histogram column), NKG2A (Z199; thick line, right histogram column) or cIg (dotted lines) followed by FITC-conjugated goat anti-mouse Ig and anti-CD3 (Cychrome conjugated) and anti-CD56 (PE conjugated; thick line, left histogram column). A gate was set on the CD56⁺CD3⁻ NK cell population within the lymphocyte gate. Note that the CD94 staining is markedly biphasic among PB-NK cells (divided into a CD94^{dim} and a CD94^{bright} subset), and that most SF-NK cells belong to a CD94^{bright} NKG2A⁺ subset, whereas only a fraction of PB-NK cells are NKG2A⁺. FIG. 8B: The percentages of CD94^{dim}, CD94^{bright} and NKG2A expressing cells within a CD56⁺CD3⁻ gated lymphocyte population were calculated (5000-10000 events within this NK cell gate were aquired). A marked increased fraction of SF-NK cells (white bars) belong to the CD94^{bright} (78.5±3.0%, n=17, p<0.001) subset when compared to PB-NK cells (black bars), which are mostly CD94^{dim} (69.2±4.9%, n=15). The increased fraction of CD94^{bright} SF-NK cells were accompanied by an increase of NKG2A⁺ cells (93.6%, n=6, p<0.001). FIG. 8C: A small subset of CD56^{bright} PB-NK cells phenotypically resembles the major SF-NK cell subset. Equal cell numbers of freshly isolated PB mononuclear cells derived from 7 healthy blood donors were pooled and immediately triple-stained with the following antibodies: control Ig (Y-axis, upper left), a cocktail of anti-KIR mAbs (DX9, DX27 and DX31 on Y-axis, upper right), anti-CD94 (DX22 on Y-axis, lower left), and anti-NKG2A (Z199 on Y-axis, lower right) followed by PE-conjugated goat anti-mouse antibodies and anti-CD3 (Cychrome conjugated) and anti-CD56 (FITC conjugated, X-axis). Approximately 10⁵ events within a lymphocyte gate were aquired to obtain at least 10³ events within the CD56^{bright} cell population and an analysis gate was set on the CD3− cells. Note that KIR expression is confined to the CD56^{dim} NK cell population whereas the CD56^{bright} NK cell population is KIR− and expresses high levels of CD94 and NKG2A.

FIGS. 9A-9C demonstrate that SF-NK cells functionally recognize HLA-E. FIG. 9A: In vitro cultured polyclonal SF-NK cell lines from two patients were used as effectors in an Alamar-blue cytotoxicity assay against untransfected 721.221 cells (HLA class I-, black bars), G_{L}-B*5801 transfected cells (721.221 cells expressing a chimeric protein where the HLA-G leader peptide has been grafted onto the HLA-B*5801 protein, hatched bars) and wild-type HLA-B*5801 transfected 721.221 cells (white bars). The E/T ratio was 1:1. FIG. 9B: The same two polyclonal SF-NK cell lines used in FIG. 8A were tested as effectors in an Alamar-blue cytotoxicity assay (E/T ratio was 1:1) against untransfected 721.221 cells (HLA class I-, white bars) and G_{L}-B*5801 transfected cells (black bars). Blocking MHC class I or CD94 with specific mAb reverse the protection conferred by HLA-E expression on G_{L}-B*5801 transfected cells. Anti-CD94 (DX22), anti-HLA class I (w6/32) or cIg was present during the cytoxicity assays at a concentration of 1 μg/ml. FIG. 9C: Tetrameric HLA-E molecules brightly stain most SF-NK cells. Freshly isolated cells from PB (left) and SF (right) of a representative RA patient were stained with control tetramers (mouse H2-K^{b} molecules conjugated to streptavidine-PE, Y-axis on upper contour plots) and HLA-E tetrameric molecules (which were refolded in the presence of a HLA-B*0701 nonamer-peptide conjugated to streptavidine-PE on Y-axis, lower contour plots) and CD56-FITC (X-axis). A gate was set on CD3-Cycrome-lymphocytes.

FIG. 10 demonstrates that CD94/NKG2A binding of self-HLA class I is the main receptor/ligand interaction protecting autologous cells from lysis by SF-NK cells. The polyclonal SF-NK and PB-NK cell lines analyzed in FIGS. 9A and 9B (patient 2), were used in a 4 hrs ⁵¹Cr-release cytotoxic assay against EBV-transformed autologous cells at an E/T ratio of 4:1. Blocking MHC class I (hatched bars) or CD94/NKG2A (black bars) with specific mAb induced killing of autologous cells but cIg (white bars) had no effect. When using SF-NK cell line as effector, similar levels of killing were observed in the presence either anti-MHC class I or anti-CD94 mAbs, suggesting that most of the self-protection is due to CD94/NKG2A interacting with HLA-class I on autologous cells. Anti-CD94 (DX22) or anti-HLA class I (w6/32), or cIg were present during the cytotoxicity assays at a concentration of 1 μg/ml.

FIG. 11 demonstrates that SF-NK cells bind to HLA-E in complex with an exemplary, VMAPRTVLL (SEQ ID NO: 3) peptide. Tetrameric HLA-E/B7sp molecules brightly stain most SF-NK cells. Freshly isolated cells from PB (left) and SF (right) of a representative RA patient were stained with control tetramers (mouse H2-Kb molecules conjugated to streptavidine-PE, Y-axis on upper contour plots) and HLA-E tetrameric molecules (which were refolded in the presence of a VMAPRTVLL(SEQ ID NO: 3) peptide) conjugated to streptavidine-PE on Y-axis, lower contour plots) and CD56-FITC (X-axis). A gate was set on CD3-Cycrome-negative lymphocytes.

FIG. 12 shows that SF-NK cells bind to HLA-E in complex with VMAPRTVLL (SEQ ID NO: 3) (B7sp) peptide but not to HLA-E in complex with QMRPVRSVL (SEQ ID NO: 2) (hsp60sp) peptide. Tetrameric HLA-E/B7sp molecules brightly stain most SF-NK-cells (upper row, middle contour plot) and a fraction of SF-T cells (lower row, middle contour plot). No staining of SF-NK cells or SF-T cells is observed with HLA-E/hsp60sp (upper row, right contour plot and lower row, right contour plot, respectively). Control tetramer staining (mouse H2-Kb molecules conjugated to streptavidine-PE) is shown to the left.

FIG. 13 demonstrates that SF-NK cells are more prone to produce IFN-gamma and TNF-alpha upon stimulation with LPS as compared to PB-NK cells of either RA patients or healthy individuals. PB and SF mononuclear cells (MC) were stimulated with LPS (10 mg/ml) over night, or with K562 (1:1 cell ratio) for 4 hrs in the presence of GolgiStop™. Cells were surface stained for CD3 and CD56 and thereafter stained intracellularly for IFN-gamma or TNF-alpha. Analysis was performed by flow cytometry.

FIG. 14 shows that SF-NK cells are more prone to produce IFN-gamma after stimulation with IL-2 as compared to PB-NK cells. PB and SF mononuclear cells (MC) were stimulated with IL-2 (200 U/ml) over night. Cells were surface stained for CD3 and CD56 and thereafter stained intracellularly for IFN-gamma or TNF-alpha. Analysis was performed by flow cytometry.

FIG. 15 demonstrates that HLA-E presenting B7 signal peptide (VMAPRTVLL) (SEQ ID NO: 3) are sufficient to inhibit NK cell IFN-gamma and TNF-alpha cytokine production. HLA-E expression was stabilized on K562 cells transfected with HLA-E*01033, by incubation with various doses of HLA-B7 signal sequence derived peptide (VMAPRTVLL) (SEQ ID NO: 3). PB and SF mononuclear cells (MC) of RA patients were then incubated with peptide stabilized K562 cells (1:1 cell ratio) for 4 hours in the presence of GolgiStop™. Cells were surface stained for CD3 and CD56 and thereafter stained intracellularly for IFN-gamma or TNF-alpha. Analysis was performed by flow cytometry.

### DESCRIPTION OF THE SPECIFIC EMBODIMENTS

The instant invention satisfies the foregoing needs and fulfills additional objects and advantages by providing novel methods and compositions for diagnosis and treatment of inflammatory diseases and conditions, autoimmune disorders, viral infection, graft rejection, and cancer, among other diseases and conditions. These compositions and methods employ a proinflammatory or anti-inflammatory binding peptide to modulate an immune response in a subject, typically a mammalian subject presenting with a disease or condition amenable to treatment according to the methods and compositions of the invention.

Peptides for use within the invention exhibit specific binding interations with a major histocompatibility complex class I (MHC class I) molecule, for example a HLA-E MHC class I molecule. Typically, these MHC I molecules will be present on an antigen presenting cell (APC). A complex between the MHC I molecule and the peptide bound in the binding cleft of the MHC I molecule forms upon exposure of the cell to the peptide. The resultant bound complex interacts with a MHC class I-specific inhibitory receptor, typically be a CD94/NKG2 cellular receptor (comprised of CD94 paired with NKG2A or its splice variant NKG2B). The interactions between the proinflammatory or anti-inflammatory binding peptide and HLA-E binding peptide (optionally involving an additional binding peptide), modulates interactions between the binding peptide/HLA-E complex and the receptor to yield novel regulation of an immune response in a population of cells expressing the inhibitory receptor.

The term “major histocompatibility complex molecule” refers to a molecule on an antigen presenting cell that has the ability to associate with the antigen to form an antigen-associated antigen presenting cell. Recognition of the antigen-associated presenting cell by the NK and T cells is mediated by the CD94/NKG2 cellular receptor.

The class I molecule, composed of a heavy chain and a noncovalently linked beta-2-microglobulin molecule, includes a cleft or crevice for receiving the proinflammatory or anti-inflammatory binding peptide. Accordingly, the peptide has a size and dimension that permits entry of the peptide into the crevice. The size and dimension of the crevice is known to those of ordinary skill in the art (F. Latron Science 257:964-967, 1992, incorporated herein by reference). Preferably, the peptide fits substantially within the crevice, but is still accessible to a NK or T cell capable of recognizing the antigen when it is associated with the class I molecule. In general, the peptide will comprise between about 4-24 amino acids in length, often between about 6-15 amino acids in length, and more commonly between about eight and ten amino acids in length. In typical embodiments, the peptide is a nonomer. Commonly, two of the amino acids of the peptide are hydrophobic residues for retaining the peptide in the crevice. The peptide may, for example, be derived from a tumor, a tissue, a viral protein or a bacterial protein.

In related embodiments of the invention, the proinflammatory or anti-inflammatory binding peptide prevents or induces NK cell activation (e.g. cytotoxicity and cytokine release) during encounter with normal and abnormal (e.g., cancerous or viral infected) cells. NK cells bearing CD94/NKG2A receptors that regulate their self-tolerance are capable of killing cells that have lost the expression of protective HLA-E molecules.

In more detailed aspects of the invention, proinflammatory binding peptides are peptides derived from a signal sequence of another MHC class I molecule. Antiinflammatory peptides are typically peptides derived from a stress-induced or stress-related protein, or a heat shock protein (hsp), for example hsp60. In contrast to classical MHC class I molecules, HLA-E displays a rather limited polymorphism, and its peptide binding cleft is primarily occupied by nonameric peptides derived from the signal sequence of certain HLA-A, -B, -C, and -G molecules (Lazetic et al., J. Immunol. 157:4741-4745, 1996, incorporated herein by reference). These peptides generally share a common motif: methionine at position 2, and leucine or isoleucine at position 9 (Arnett et al., Arthritis Rheum. 31:315-324, 1988, incorporated herein by reference). In addition, the peptides also exhibit a third common motif element, which is a proline residue at position 4. Peptides sharing this motif or similar structure are useful as candidate peptides for screening within the invention to identify operable proinflammatory and anti-inflammatory binding peptides capable of binding HLA-E and mediating regulation of immune responses by modulating interactions with CD94/NKG2 cellular receptors.

In certain embodiments of the invention, the HLA-E binding peptide is derived from a signal sequence of a stress induced protein. For example, exemplary peptides may be selected from the stress induced peptide hsp60. In one embodiment of the invention the hsp 60 peptide is a nonamer. Examples of preferred peptides are (standard one letter code) VMAPVTVLL (SEQ ID NO: 4) and QMRPRSRVL (SEQ ID NO: 2).

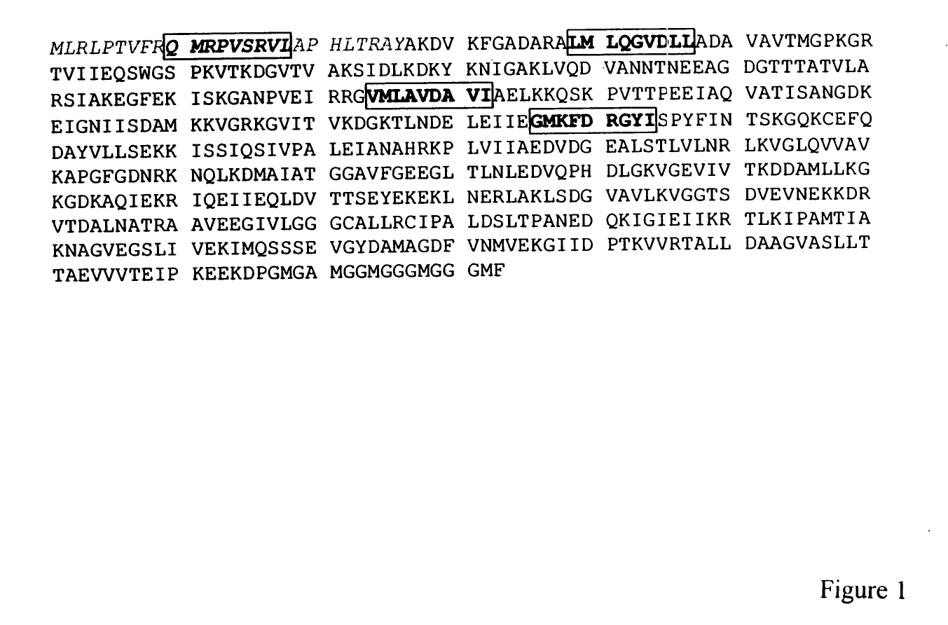

To identify peptides derived from human hsp60with a potential to bind HLA-E, the full length amino acid sequence of hsp60 was scanned for peptides displaying an HLA-E permnissive motif (methionine at position 2 followed by either a leucine or isoleucine at position 9 at the C-terminus). Among four such peptides identified (FIG. 1; Table 1), one (QMRPVSRVL (SEQ ID NO: 2), designated hsp60sp) was initially selected based on its location within the hsp60 leader sequence. In addition, hsp60sp not only bears a methionine at position 2 and a leucine at position 9, but also shares amino acids at position 4 and 8 in common with some peptides known to efficiently bind to HLA-E (Table 1). In particular, four out of the nine amino acids in hsp60sp are shared with some peptides found in HLA class I leader sequences (e.g., HLA-A*0201, and -A*3401, Table 1).

TABLE I

Peptide sequence comparisons between HLA
class I molecules and hsp60

| | | | signal |
|---|---|---|---|
| | | | peptide (SP) |
| | | peptide | mature |
| Protein | (residues) | sequence | protein (P) |
| HLA-A*0201 | (3-11) | VMAPRTLVL | SP |
| | | (SEQ ID NO:4) | |
| HLA-A*0301 | (3-11) | VMAPRTLLL | SP |
| | | (SEQ ID NO:5) | |
| HLA-A*3401 | (3-11) | IMAPRTLVL | SP |
| | | (SEQ ID NO:6) | |
| HLA-B*0701 | (3-11) | VMAPRTVLL | SP |
| | | (SEQ ID NO:3) | |
| HLA-Cw*0102 | (3-11) | VMAPRTLIL | SP |
| | | (SEQ ID NO:7) | |
| HLA-G*0101 | (3-11) | VMAPRTLFL | SP |
| | | (SEQ ID NO:8) | |
| hsp60sp | (10-18) | Q M R PVSRVL | SP |
| | | (SEQ ID NO:2) | |
| hsp60.2 | (39-47) | L MLQGVDLL | P |
| | | (SEQ ID NO:9) | |
| hsp60.3 | (144-152) | VMLAVDAVI | P |
| | | (SEQ ID NO:10) | |
| hsp60.4 | (216-224) | G MKFDRGYI | P |
| | | (SEQ ID NO:11) | |

Diseases and conditions amenable to treatment and diagnosis according to the methods and compositions of the invention include, but are not limited to, rheumatoid arthritis, juvenile arthritis, Chron's disease, ulcerative colitis, acute myeloid leukemia, multiple sclerosis, insulin-dependent diabetes mellitus, systemic lupus erythematosus, SjUgren syndrome, Basedow disease, Hashimoto disease, autoimmune hemolytic anemia, cancer (e.g., ovarial cancer), cardiomyopathy, early cardiovascular disease, artherosclerosis, hypertension, Hodgkin's disease, and transplant or graft rejection. In exemplary reports supporting this broad range of applicability of the invention, NK cells have been implicated with an important role in down-regulating TH1-mediated colitis by controlling the responses of effector T cells in a perforin dependent manner (Fort et al., J. Immunol. 161:3256-3261, 1998, incorporated herein by reference). In experimental autoimmune encephalomyelitis (EAE), a model for human multiple sclerosis (MS), administration of the NK cell stimulatory compound linomide can protect mice from developing disease, and in the same model depletion of NK cells led to increased production of TH1 cytokines and an exacerbation of disease (Matsumoto et al., Eur. J. Immunol. 28:1681-1688, 1998; Zhang et al., J. Exp. Med. 186:1677-1687, 1997, each incorporated herein by reference). These reports suggest that the presence of NK cells is beneficial for the protection against prototype TH1-mediated diseases. In contrast, a pathogenic role for NK cells was suggested in a murine model of asthma, a prototype TH2-mediated disease, where depletion of NK cells protected mice from developing allergen-induced inflammation in the airway epithelium

To identify additional peptides derived from heat shock proteins (hsps) and other proteins, similar rational design and screening methods are employed as were employed above for hsp60. Candidate hsps with a potential to bind HLA-E may be identified based on the structural considerations described herein, and their known activity in mediating onset or exacerbation of disease states. As illustrated below in Tables 2 and 3, below many hsps are implicated in serious diseases and conditions amenable to treatment according to the methods and compositions of the invention. To identify candidate proinflammatory or anti-inflammatory binding peptides from these subject proteins with known deleterious activities associated with disease, the full length amino acid sequence of the protein is scanned for peptides displaying an HLA-E permissive motif (e.g., methionine at position 2 followed by either a leucine or isoleucine at position 9 at the C-terminus). Candidate peptides thus identified are evaluated and screened according to the methods set forth herein.

TABLE 2
Reports describing increased levels
of hsp60 associated with disease

| Disease | Reference |
|---|---|
| Rheumatoid arthritis | Boog C J et al. J Exp Med. 1992 Jun |
| Juvenile arthritis | 1; 175(6): 1805-10. |
| | A. Karlsson-Parra et al. Scand. J. Immunol. |
| | 1990, 31: 283-288 |
| Chron's disease and | Peetermans W E et al. |
| Ulcerative colitis | Gastroenterology. 1995 Jan; 108(1): 75-82. |
| | Baca-Estrada ME et al. Dig Dis Sci. 1994 |
| | Mar; 39(3): 498-506. |
| Acute myeloid leukemia | Chant I D et al. Br J Haematol. 1995 |
| | May; 90(1): 163-8. |
| Ovarial cancer | Schneider J et al. Anticancer Res. 1999 |
| | May-Jun; 19(3A): 2141-6. |
| Cardiomyopathy | Latif N et al |
| | Basic Res Cardiol. 1999 Apr; 94(2): 112-9. |
| Early cardiovascular | Pockley A G et al. Hypertension. 2000 |
| disease Artherosclerosis | Aug; 36(2): 303-7. |
| | Xu Q et al. Circulation. 2000 Jul |
| | 4; 102(1): 14-20. |
| Hodgkin's disease | Hsu P L et al. Cancer Res. 1998 Dec |
| | 1; 58(23): 5507-13. |
| Transplant rejection | Alevy Y G et al. Transplantation. 1996 Mar |
| | 27; 61(6): 963-7. |

TABLE 3

Involvement of heat shock proteins
(hsp) in disease

| | | | Protein |
|---|---|---|---|
| | | | Acc. No. |
| | | | HLA-E |
| Hsp | | | binding |
| (chape- | | | motifs |
| rone) | Disease | Refs | (position) |
| Human | RA | 1 | P25685 |
| hsp40 | | | KMKISHKRL |
| | | | (SEQ ID NO:12) |
| | | | (182-190) |
| Human | Juvenile arthritis | 2, 3, 4, | P10809 |
| hsp60 | Multiple sclerosis | 5, 6 | QMRPVSRVL |
| | Atherosclerosis | | (SEQ ID NO:13) |
| | IDDM, | | (10-18) |
| | Kawasaki disease | | LMLQGVDLL |
| | Psoriasis | | (SEQ ID NO:14) |
| | Cancer | | (39-47) |
| | | | VMLAVDAVI |
| | | | (SEQ ID NO:15) |
| | | | (144-152) |
| | | | GMKFDRGYI |
| | | | (SEQ ID NO:11) |
| | | | (216-224) |
| Human | MS | 7, 8, 9 | P08107 |
| hsp70 | Cancers | | AMTKDNNLL |
| | RA | | (448-456) |
| Human | RA | 10 | P11021 |
| grp78 | | | TMKPVQKVL |
| (Bip) | | | (SEQ ID NO:17) |
| | | | (338-346) |
| Human | RA | 9 | P07900 |
| hsp90 | | | PMGRGTKVI |
| | | | (SEQ ID NO:19) |
| | | | (179-187) |
| | | | I MDNCEELI |
| | | | (SEQ ID NO:20) |
| | | | (370-378) |
| | | | EMLQQSKIL |
| | | | (SEQ ID NO:21) |
| | | | (401-409) |
| | | | RMKENQKH I |
| | | | (SEQ ID NO:22) |
| | | | (483-491) |
| | | | R MIKLGLGI |
| | | | (SEQ ID NO:23) |
| | | | (690-698) |
| gp96 | Cancer | 11 | XP_083864 |
| | | | MMKLIINSL |
| | | | (SEQ ID NO:24) |
| | | | (85-93) |
| | | | RMKEKQDKI |
| | | | (SEQ ID NO:25) |
| | | | (530-538) |
| | | | RMLRLSLNI |
| | | | (SEQ ID NO:26) |
| | | | (741-749) |

References cited above
Albani S. et al. Nat Med. May;1(5):448-52. 1995
de Graeff-Meeder, E.R. et al. Clin Exp Rheumatol 11 Suppl 9, S25-28. 1993
Xu, Q. et al. Arterioscier Thromb 13:1763-1769. 1993
Yokota, S. et al. Clin Immunol Immunopathol. 67:163-170. 1993
Rambukkana, A. et al. J Invest Dermatol 100, 87-92. 1993
Raz I. et al. Lancet. November 24;358(9295):1749-53. 2001
Salvetti M et al. J Neuroimmunol. April;65(2):143-53. 1996
Jenkins SC et al. Tissue Antigens. July;56(1):38-44. 2000
Hayem G. et al. Ann Rheum Dis. May;58(5):291-6. 1999.
Blass Set al. Arthritis Rheum. April;44(4):761-71. 2001
Somersan S. et al. J Immunol. November 1;167(9):4844-52. 2001

Each of the peptide sequences identified in Table 3 above is considered to be a useful candidate proinflammatory or anti-inflammatory binding peptide for use within the diagnostic and therapeutic methods of the invention.

In addition, a variety of other proteins have been analyzed to determine candidate proinflammatory or anti-inflammatory binding peptides for use within the invention. In one such exemplary analysis, a BLAST search was conducted that identified a stretch of nucleotides within Homo sapiens beta defensin 2 (HBD2) gene that shows 85% amino acid identity with the human hsp60 leader sequence. The reading frame is reversed (−2) starting from position HBD2: 718 to 659, and show 85% homology with the hsp60-leader peptide.

BLAST Search Results:

gi|3818536|gb|AF071216.1|AF071216, complete cds
Score=37.0 bits (84), Expect=0.34
Identities= 17/20 (85%), Positives= 18/20 (90%)

Frame=−2

Query human hsp60sp:

(SEC ID NO:27)

``1 MLRLPTVFRQMRPVSRVLAP 20
identities

(SEC ID NO:28)

ML LPTVF QMRPVSR + LAP
HBD2:

(SEC ID NO:29)

718 MLPLPTVFHQMRPVSRLLAP 659

From these and other subject proteins and peptide sequences, the amino acid sequence is scanned for peptides displaying an HLA-E permissive motif. Candidate peptides thus identified are evaluated and screened according to the methods set forth herein. Table 4 sets forth a large assemblage of candidate HLA-E binding peptides for use within the invention.

TABLE 4

PUTATIVE HLA-E BINDING PEPTIDES DERIVED
FROM NON-MHC HUMAN PROTEINS CARRYING
POS. 2: M, POS. 4. P AND POS. 9:I OR L

| | ACC. NO. | | |
|---|---|---|---|
| | SWISS | | |
| PROTEIN | PROT. | SEQUENCE | POSITION |
| INTER-ALPHA TRYPSIN | P19827 | AMGPRGLLL | 4-12 |
| INHIBITORY HEAVY | | (SEQ ID | (SIGNAL |
| CHAIN H1 PRECURSOR | | NO:30) | PEPTIDE) |
| PLASMINOGEN | P05121 | QMSPALTCL | 2-10 |
| ACTIVATOR | | (SEQ ID | (SIGNAL |
| INHIBITOR-1 | | NO:31) | PEPTIDE) |
| | | GMAPALRHL | 93-101 |
| | | (SEQ ID | |
| | | NO:32) | |
| CELL SURFACE A33 | Q99795 | KMWPVLWTL | 4-12 |
| ANTIGEN PRECURSOR | | (SEQ ID | (SIGNAL |
| | | NO:33) | PEPTIDE) |
| ACROSIN PRECURSOR | P10323 | EMLPTAILL | 3-11 |
| | | (SEQ ID | (SIGNAL |
| | | NO:34) | PEPTIDE) |
| CLASS II | P28067 | QMLPLLWLL | 13-21 |
| HISTOCOMPATIBILITY | | (SEQ ID | (SIGNAL |
| ANTIGEN | | NO:35) | PEPTIDE) |
| BRAIN SPECIFIC | Q9UK28 | LMPPPLLLL | 6-14 |
| MEMBRANE- | | (SEQ ID | (SIGNAL |
| ANCHORED PROTEIN | | NO:36) | PEPTIDE) |
| PRECURSOR | | | |
| GC-RICH SEQUENCE | P16383 | AMAPRSRLL | 60-68 |
| DNA BINDINING | | (SEQ ID | |
| FACTOR | | NO:37) | |
| T BOX TRANSCRIPTION | P57082 | TMMPRLPTL | 450-458 |
| FACTOR | | (SEQ ID | |
| | | NO:38) | |
| RETINOBLASTOMA | P06400 | KMTPRSRIL | 824-832 |
| ASSOCIATED PROTEIN | | (SEQ ID | |
| | | NO:39) | |
| FATTY ACID | P49327 | TMDPQLRLL | 74-82 |
| SYNTHASE | | (SEQ ID | |
| | | NO:40) | |
| TRANSITIONAL | P55072 | GMTPSKGVL | 507-515 |
| ENDOPLASMIC | | (SEQ ID | |
| RETICULUM PROTEIN | | NO:41) | |
| CARBOXYPEPTIDASE M | P14384 | PMIPLYRNL | 411-419 |
| PRECURSOR | | (SEQ ID | |
| | | NO:42) | |
| THROMBOXANE-A | P24557 | IMVPLARIL | 235-243 |
| SYNTHASE | | (SEQ ID | |
| | | NO:43) | |
| INTERFERON | Q02556 | DMAPLRSKL | 356-364 |
| CONSENSUS SEQUENCE | | (SEQ ID | |
| BINDING PROTEIN | | NO:44) | |
| LYMPHOCYTE | P13796 | PMNPNTNDL | 145-153 |
| CYTOSOLIC PROTEIN | | (SEQ ID | |
| | | NO:45) | |
| RYANODINE RECEPTOR | P21817 | QMGPQEENL | 2169-2177 |
| | | EMCPDIPVL | 3238-3246 |
| | | YMEPALRCL | 4639-4647 |
| | | (SEQ ID | |
| | | NO:46) | |
| PROTEASOME SUBUNIT | P25787 | GMGPDYRVL | 77-85 |
| ALPHA TYPE 2 | | (SEQ ID | |
| | | NO:47) | |
| 60S RIBOSOMAL | P08526 | FMKPGKVVL | 3-11 |
| PROTEIN | | (SEQ ID | |
| | | NO:48) | |
| HETEROGENEOUS | P52272 | RMGPGIDRL | 403-411 |
| NUCLEAR | | (SEQ ID | |
| RIBONUCLEOPROTEIN | | NO:49) | |
| SERYL-TRNA | P49591 | FMPPGLQEL | 458-466 |
| SYNTHETASE | | (SEQ ID | |
| | | NO:50) | |
| TELOMERASE REVERSE | O14746 | QMRPLFLEL | 388-396 |
| TRANSCRIPTASE | | (SEQ ID | |
| | | NO:51) | |
| PROTEIN DISULFIDE | P13667 | VMDPKKDVL | 539-547 |
| ISOMERASE | | (SEQ ID | |
| | | NO:52) | |
| VINCULIN | P18206 | MMGPYRQDL | 532-540 |
| | | (SEQ ID | |
| | | NO:53) | |
| WILMS' TUMOR | P19544 | RMFPNAPYL | 126-134 |
| PROTEIN | | (SEQ ID | |
| | | NO:54) | |
| ZINC FINGER PROTEIN | O43670 | GMPPGIPPL | 155-163 |
| | | (SEQ ID | |
| | | NO:55) | |
| RNA HELICASE | O43738 | IMNPSYYNL | 828-836 |
| | | (SEQ ID | |
| | | NO:56) | |
| CPSB | Q9P2I0 | QMKPRQLII | 352-360 |
| | | (SEQ ID | |
| | | NO:57) | |
| TIGHT JUNCTION | O95049 | QMKPVKSVL | 189-197 |
| PROTEIN ZO-3 | | (SEQ ID | |
| | | NO:58) | |
| PHOSPHOENOLPYRUVA | Q16822 | SMGPVGSPL | 163-171 |
| TE CARBOXYKINASE | | (SEQ ID | |
| | | NO:59) | |
| ATP-BINDING | Q99758 | GMDPVARRL | 1544-1552 |
| CASSETTE, SUB- | | (SEQ ID | |
| FAMILY A, MEMBER 3 | | NO:60) | |
| ACTIN CROSS-LINKING | Q9UPN3 | TMPPVGTDL | 4180-4188 |
| FAMILY PROTEIN 7 | | (SEQ ID | |
| | | NO:61) | |
| POTENTIAL | O60312 | LMTPVAALL | 943-951 |
| PHOSPHOLIPID- | | (SEQ ID | |
| TRANSPORTING | | NO:62) | |
| ATPASE | | | |
| CHROMODOMAIN- | O14646 | RMRPVKAAL | 1420-1428 |
| HELICASE-DNA- | | (SEQ ID | |
| BINDING PROTEIN 1 | | NO:63) | |
| CHROMODOMAIN- | O14647 | RMRPVKKAL | 1475-1483 |
| HELICASE-DNA- | | (SEQ ID | |
| BINDING PROTEIN 2 | | NO:64) | |
| CYTOCHROME P450 | P08686 | SMEPVVEQL | 134-142 |
| XXIB | | (SEQ ID | |
| | | NO:65) | |
| DNA LIGASE III | P49916 | LMTPVQPML | 390-398 |
| | | (SEQ ID | |
| | | NO:66) | |
| LYSOSPHINGOLIPID | Q99500 | AMNPVIYTL | 292-300 |
| RECEPTOR | | (SEQ ID | |
| | | NO:67) | |
| GC-RICH SEQUENCE | Q9Y5B6 | EMTPVTIDL | 382-390 |
| DNA-BINDING FACTOR | | (SEQ ID | |
| HOMOLOG | | NO:68) | |
| RAP1 GTPASE-GDP | P52306 | EMPPVQFKL | 414-422 |
| DISSOCIATION | | (SEQ ID | |
| STIMULATOR 1 | | NO:69) | |
| HOST CELL FACTOR C1 | P51610 | RMAPVCESL | 1253-1261 |
| (HCF) | | (SEQ ID | |
| | | NO:70) | |
| LEUKOTRIENE A-4 | P09960 | SMHPVTAML | 594-602 |
| HYDROLASE | | (SEQ ID | |
| | | NO:71) | |
| NEUROPEPTIDE Y | P49146 | KMGPVLCHL | 117-125 |
| RECEPTOR TYPE 2 | | (SEQ ID | |
| | | NO:72) | |
| OLFACTORY RECEPTOR | P47893 | EMQPVVFVL | 25-33 |
| 3A2 | | (SEQ ID | |
| | | NO:73) | |
| PAX-7 | P23759 | HMNPVSNGL | 374-382 |
| | | (SEQ ID | |
| | | NO:74) | |
| PROTOCADHERIN 15 | Q96QU1 | LMDPVKQML | 86-94 |
| PRECURSOR | | (SEQ ID | |
| | | NO:75) | |
| PERILIPIN (PER1) | O60240 | SMEPVVRRL | 72-80 |
| | | (SEQ ID | |
| | | NO:76) | |
| 26S PROTEASOME NON- | Q9UNM6 | LMHPVLESL | 217-225 |
| ATPASE REGULATORY | | (SEQ ID | |
| SUBUNIT 13 | | NO:77) | |
| MELANOCYTE- | Q04671 | TMIPVLLNL | 747-755 |
| SPECIFIC | | (SEQ ID | |
| TRANSPORTER | | NO:78) | |
| PROTEIN | | | |
| REGULATOR OF | P18754 | SMVPVQVQL | 162-170 |
| CHROMOSOME | | (SEQ ID | |
| CONDENSATION | | NO:79) | |
| RING FINGER PROTEIN | Q9Y3C5 | CMEPVDAAL | 139-147 |
| 11 | | (SEQ ID | |
| | | NO:80) | |
| SIDEROFLEXIN 2 | Q96NB2 | FMVPVACGL | 277-285 |
| | | (SEQ ID | |
| | | NO:81) | |
| SGT1 PROTEIN | O95905 | VMAPVDVDL | 590-598 |
| | | (SEQ ID | |
| | | NO:82) | |
| HYPOTHETICAL | Q14139 | VMIPVFDIL | 275-283 |
| PROTEIN KIAA0126 | | (SEQ ID | |
| | | NO:83) | |
| HYPOTHETICAL ZINC | Q9Y2H8 | QMAPVQKNL | 62-70 |
| FINGER PROTEIN | | (SEQ ID | |
| | | NO:84) | |
| ZINC FINGER PROTEIN | Q9HBT7 | LMRPVQKEL | 179-187 |
| ZNF287 | | (SEQ ID | |
| | | NO:85) | |
| HUMAN | Q14728 | EMAPWFALL | 201-209 |
| TETRACYCLINE | | (SEQ ID | |
| TRANSPORTER-LIKE | | NO:86) | |
| PROTEIN | | | |
| HUMAN KU8O | Q9W627 | LMLPDFDLL | 82-90 |
| AUTOANTIGEN | | (SEQ ID | |
| HOMOLOGUE | | NO:87) | |
| ADAPTER-RELATED | O00203 | TMDPDHRLL | 292-300 |
| PROTEIN COMPLEX 3 | | (SEQ ID | |
| BETA 1 SUBUNIT | | NO:88) | |
| ADAPTER-RELATED | Q13367 | VMDPDHRLL | 292-300 |
| PROTEIN COMPLEX 3 | | (SEQ ID | |
| BETA 2 SUBUNIT | | NO:89) | |
| CYCLIN A1 | P78396 | LMEPPAVLL | 455-463 |
| | | (SEQ ID | |
| | | NO:90) | |
| COMPLEMENT C5 | P01031 | NMVPSSRLL | 533-541 |
| PRECURSOR | | (SEQ ID | |
| | | NO:91) | |
| CYTOCHROME P450 4F2 | P78329 | WMGPISPLL | 91-99 |
| | | (SEQ ID | |
| | | NO:92) | |
| CYTOCHROME P450 4F12 | Q9HCS2 | AMSPWLLLL | 15-23 |
| | | (SEQ ID | |
| | | NO:93) | |
| G PROTEIN-COUPLED | Q8WTQ7 | DMKPENVLL | 316-324 |
| RECEPTOR KINASE | | (SEQ ID | |
| GRK7 | | NO:94) | |
| GLUTATHIONE S- | Q16772 | RMEPIRWLL | 15-23 |
| TRANSFERASE A3-3 | | (SEQ ID | |
| | | NO:95) | |
| SOLUTE CARRIER | P14672 | AMGPYVFLL | 444-452 |
| FAMILY 2 | | (SEQ ID | |
| | | NO:96) | |
| ATP-DEPENDENT DNA | P13010 | LMLPDFDLL | 82-90 |
| HELICASE II | | (SEQ ID | |
| | | NO:97) | |
| MITOGEN-ACTIVATED | Q99683 | LMQPNFELL | 237-245 |
| PROTEIN KINASE | | (SEQ ID | |
| KINASE KINASE 5 | | NO:98) | |
| MUELLERIAN | P03971 | RMTPALLLL | 244-252 |
| INHIBITING FACTOR | | (SEQ ID | |
| PRECURSOR | | NO:99) | |
| CANALICULAR | O15438 | EMGPYPALL | 831-839 |
| MULTISPECIFIC | | (SEQ ID | |
| ORGANIC ANION | | NO:100) | |
| TRANSPORTE | | | |
| METASTASIS- | Q13330 | HMGPSRNLL | 614-622 |
| ASSOCIATED PROTEIN | | (SEQ ID | |
| MTA1 | | NO:101) | |
| SODIUM/HYDROGEN | Q92581 | LMRPLWLLL | 24-32 |
| EXCHANGER 6 | | (SEQ ID | |
| | | NO:102) | |
| PYD-CONTAINING | Q9NX02 | VMLPKAALL | 325-333 |
| PROTEIN 2 | | (SEQ ID | |
| | | NO:103) | |
| PYD-CONTAINING | Q96MN2 | KMLPEASLL | 268-276 |
| PROTEIN 4 | | (SEQ ID | |
| | | NO:104) | |
| PANNEXIN 3 | Q96QZ0 | EMLPAFDLL | 306-314 |
| | | (SEQ ID | |
| | | NO:105) | |
| PEROXISOME | O43933 | WMQPSVVLL | 653-661 |
| BIOGENESIS FACTOR 1 | | (SEQ ID | |
| | | NO:106) | |
| LONG TRANSIENT | O94759 | TMDPIRDLL | 618-626 |
| RECEPTOR POTENTIAL | | (SEQ ID | |
| CHANNEL 2 | | NO:107) | |
| UTEROGLOBIN- | Q96PL1 | FMDPLKLLL | 45-53 |
| RELATED PROTEIN 1 | | (SEQ ID | |
| PRECURSOR | | NO:108) | |
| WILLIAMS-BEUREN | Q9NP71 | PMAPPTALL | 417-425 |
| SYNDROME | | (SEQ ID | |
| CHROMOSOME REGION | | NO:109) | |
| 14 | | | |
| HYPOTHETICAL | Q15053 | AMCPIAMLL | 41-49 |
| PROTEIN KIAA0040 | | (SEQ ID | |
| | | NO:110) | |

### REFERENCES

1. Braud, V. M., D. S. Allan, C. A. O'Callaghan, K. Soderstrom, A. D'Andrea, G. S. Ogg, S. Lazetic, N. T. Young, J. I. Bell, I H. Phillips, L. L. Lanier, and A. I McMichael. 1998. HLA-E binds to natural killer cell receptors CD94/NKG2A, B and C [see comments]. Nature 391:795.
2. Kleinau, S., K. Soderstrom., R. Kiessling, and L. Klareskog. 1991. A monoclonal antibody to the mycobacterial 65 kDa heat shock protein (ML 30) binds to cells in normal and arthritic joints of rats. Scand J Immunol 33:195.
3. Karlsson-Parra, A., K. Soderstrom, M. Ferm, I Ivanyi, R. Kiessling, and L. Klareskog. 1990. Presence of human 65 kD heat shock protein (hsp) in inflamed joints and subcutaneous nodules of RA patients [corrected and republished with original paging, article originally printed in Scand J Immunol 1990 March; 31(3)-.283-8]. ScandJ Immunol 31:283.
4. Boog, C. J. P., E. R. de Graeff-Meeder, M. A. Lucassen, R. R. van der Zee, M. M. Voorhorst-Ogink, P. I S. van Kooten, H. J. Geutz, and W. van Eden. 1992. Two monoclonal antibodies generated against human hsp60 show reactivity with synovial membranes of patients with juvenile chronic arthritis. J Exp. Med 175:1805.
5. Lo, W.-F. et al. Molecular mimicry mediated by N4HC class Ib molecules after infection with Gram-negative pathogens. Nature Med 6, 215-218 (2000)
6. Kraft, J. R. et al. Analysis of Qa-Ib peptide binding specificity and the capacity of CD94/NKG2A to discriminate between Qa-I-peptide complexes. J Exp. Med 192, 613-623(2000).

By the use of hsp60 signal peptides or other proinflammatory HLA-E binding peptides (e.g., from stress proteins, heat shock proteins or other proteins as disclosed herein), and analogs thereof, with strong capacity to bind HLA-E and which potentially can compete out protective MHC class I-peptides in the cleft of HLA-E, a novel therapeutic tool can be developed to induce the activation of NK cells and to lower the threshold for activation of CD94-NKG2A expressing CTLs against tumor cells that have escaped immune detection on the basis of retained protective HLAE expression. These compositions and methods involve exposing a tumor cell or cancerous tissue in a patient to a therapeutically effective amount of a proinflammatory binding peptide that will thereby prevent or inhibit growth of the tumor cell or cancerous tissue.

Related methods apply to treatment of viral infected cells. Exposure of such infected cells to a therapeutically effective amount of a a proinflammatory binding peptide results in the peptide competing out protective MHC class I-peptides in the cleft of HLA-E, to induce activation of NK cells and lower the threshold for activation of CD94-NKG2A expressing CTLs against viral infected cells that may otherwise have escaped immune detection.

Peptide Analogs and Mimetics

Included within the definition of biologically active peptides for use within the invention are natural or synthetic, therapeutically or prophylactically active, peptides (comprised of two or more covalently linked amino acids), peptide analogs, and chemically modified derivatives or salts of active peptides. Often, the peptides are muteins that are readily obtainable by partial substitution, addition, or deletion of amino acids within a naturally occurring or native (e.g., wild-type, naturally occurring mutant, or allelic variant) peptide sequence. Additionally, biologically active fragments of native peptides are included. Such mutant derivatives and fragments substantially retain the desired biological activity of the native peptide. In the case of peptides having carbohydrate chains, biologically active variants marked by alterations in these carbohydrate species are also included within the invention.

In additional embodiments, peptides for use within the invention may be modified by addition or conjugation of a synthetic polymer, such as polyethylene glycol, a natural polymer, such as hyaluronic acid, or an optional sugar (e.g. galactose, mannose), sugar chain, or nonpeptide compound. Substances added to the peptide by such modifications may specify or enhance binding to certain receptors or antibodies or otherwise enhance the mucosal delivery, activity, half-life, cell- or tissue-specific targeting, or other beneficial properties of the peptide. For example, such modifications may render the peptide more lipophilic, e.g., such as may be achieved by addition or conjugation of a phospholipid or fatty acid. Further included within the methods and compositions of the invention are peptides prepared by linkage (e.g., chemical bonding) of two or more peptides, protein fragments or functional domains (e.g., extracellular, transmembrane and cytoplasmic domains, ligand-binding regions, active site domains, immunogenic epitopes, and the like)—for example fusion peptides recombinantly produced to incorporate the functional elements of a plurality of different peptides in a single encoded molecule.

Biologically active peptides for use within the methods and compositions of the invention thus include native or “wild-type” peptides and naturally occurring variants of these molecules, e.g., naturally occurring allelic variants and mutant proteins. Also included are synthetic, e.g., chemically or recombinantly engineered, peptides, as well as peptide and protein “analogs” and chemically modified derivatives, fragments, conjugates, and polymers of naturally occurring peptides. As used herein, the term peptide “analog” is meant to include modified peptides incorporating one or more amino acid substitutions, insertions, rearrangements or deletions as compared to a native amino acid sequence of a selected peptide. Peptide and protein analogs thus modified exhibit substantially conserved biological activity comparable to that of a corresponding native peptide, which means activity (e.g., specific binding to a HLA-E molecule, or to a cell expressing HLA-E, interaction of a peptide/HLA-E complex with a CD94/NKG2 cellular receptor, etc.) levels of at least 50%, typically at least 75%, often 85%-95% or greater, compared to activity levels of a corresponding native protein or peptide.

Fusion polypeptides between proinflammatory or anti-inflammatory binding peptide and other homologous or heterologous peptides are also provided. Many growth factors and cytokines are homodimeric entities, and a repeat construct of peptide linked to form “cluster peptides” will yield various advantages, including lessened susceptibility to proteolytic degradation. Various alternative multimeric constructs comprising peptides of the invention are also provided. In one embodiment, various polypeptide fusions are provided as described in U.S. Pat. Nos. 6,018,026 and 5,843,725, by linking one or more proinflammatory or anti-inflammatory binding peptides of the invention with a heterologous, multimerizing polypeptide, for example, immunoglobulin heavy chain constant region, or an immunoglobulin light chain constant region. The biologically active, multimerized polypeptide fusion thus constructed can be a hetero- or homo-multimer, e.g., a heterodimer or homodimer, which may each comprise one or more distinct proinflammatory or anti-inflammatory binding peptide(s) of the invention. Other heterologous polypeptides may be combined with the peptide to yield fusions comprising, e.g., a hybrid protein exhibiting heterologous (e.g., CD4) receptor binding specificity. Likewise, heterologous fusions may be constructed exhibit a combination of properties or activities of the derivative proteins. Other typical examples are fusions of a reporter polypeptide, e.g., CAT or luciferase, with a peptide of the inveniton, to facilitate localization of the fused protein (see, e.g., Dull et al., U.S. Pat. No. 4,859,609, incorporated herein by reference). Other gene/protein fusion partners useful in this context include bacterial beta-galactosidase, trpE, Protein A, beta-lactamase, alpha amylase, alcohol dehydrogenase, and yeast alpha mating factor (see, e.g., Godowski et al., Science 241:812-816, 1988, incorporated herein by reference).

The present invention also contemplates the use of proinflammatory or anti-inflammatory binding peptides modified by covalent or aggregative association with chemical moieties. These derivatives generally fall into the three classes: (1) salts, (2) side chain and terminal residue covalent modifications, and (3) adsorption complexes, for example with cell membranes. Such covalent or aggregative derivatives are useful for various purposes, for example as immunogens, as reagents in immunoassays, or in purification methods such as for affinity purification of ligands or other binding ligands. For example, a proinflammatory or anti-inflammatory binding peptide can be immobilized by covalent bonding to a solid support such as cyanogen bromide-activated Sepharose, by methods which are well known in the art, or adsorbed onto polyolefin surfaces, with or without glutaraldehyde cross-linking, for use in the assay or purification of antibodies that specifically bind the proinflammatory or anti-inflammatory binding peptide. The proinflammatory or anti-inflammatory binding peptide can also be labeled with a detectable group, for example radioiodinated by the chloramine T procedure, covalently bound to rare earth chelates, or conjugated to another fluorescent moiety for use in diagnostic assays.

For purposes of the present invention, the term biologically active peptide “analog” further includes derivatives or synthetic variants of a native peptide, such as amino and/or carboxyl terminal deletions and fusions, as well as intrasequence insertions, substitutions or deletions of single or multiple amino acids. Insertional amino acid sequence variants are those in which one or more amino acid residues are introduced into a predetermined site in the protein. Random insertion is also possible with suitable screening of the resulting product. Deletional variants are characterized by removal of one or more amino acids from the sequence. Substitutional amino acid variants are those in which at least one residue in the sequence has been removed and a different residue inserted in its place.

Where a native peptide is modified by amino acid substitution, amino acids are generally replaced by other amino acids having similar, conservatively related chemical properties such as hydrophobicity, hydrophilicity, electronegativity, small or bulky side chains, and the like. Residue positions which are not identical to the native peptide sequence are thus replaced by amino acids having similar chemical properties, such as charge or polarity, where such changes are not likely to substantially effect the properties of the peptide analog. These and other minor alterations will typically substantially maintain biological properties of the modified peptide, including biological activity (e.g., binding to an adhesion molecule, or other ligand or receptor), immunoidentity (e.g., recognition by one or more monoclonal antibodies that recognize a native peptide), and other biological properties of the corresponding native peptide.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine.

The term biologically active peptide analog further includes modified forms of a native peptide incorporating stereoisomers (e.g., D-amino acids) of the twenty conventional amino acids, or unnatural amino acids such as α,α-disubstituted amino acids, N-alkyl amino acids, lactic acid. These and other unconventional amino acids may also be substituted or inserted within native peptides useful within the invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and imino acids (e.g., 4-hydroxyproline). In addition, biologically active peptide analogs include single or multiple substitutions, deletions and/or additions of carbohydrate, lipid and/or proteinaceous moieties that occur naturally or artificially as structural components of the subject peptide, or are bound to or otherwise associated with the peptide.

To facilitate production and use of peptide and protein analogs within the invention, reference can be made to molecular phylogenetic studies that characterize conserved and divergent protein structural and functional elements between different members of a species, genus, family or other taxonomic group (e.g., between different stress induced or heat shock proteins, family members, allelic variants, and/or naturally occurring mutants, including between homologous proteins found in different species, such as human, murine, rat and/or bovine). In this regard, available studies will provide detailed assessments of structure-function relationships on a fine molecular level for modifying the majority of peptides disclosed herein to facilitate production and selection of operable peptide and protein analogs. These studies include, for example, detailed sequence comparisons identifying conserved and divergent structural elements among, for example, multiple isoforms or species or allelic variants of a subject proinflammatory or anti-inflammatory binding peptide. Each of these conserved and divergent structural elements facilitate practice of the invention by pointing to useful targets for modifying native peptides to confer desired structural and/or functional changes.

In the context, existing sequence alignments may be analyzed and conventional sequence alignment methods may be employed to yield sequence comparisons for analysis, for example to identify corresponding protein regions and amino acid positions between protein family members within a species, and between species variants of a protein of interest. These comparisons are useful to identify conserved and divergent structural elements of interest, the latter of which will often be useful for incorporation in a biologically active peptide to yield a functional analog thereof. Typically, one or more amino acid residues marking a divergent structural element of interest in a different reference peptide sequence is incorporated within the functional peptide analog. For example, a cDNA encoding a native proinflammatory or anti-inflammatory binding peptide may be recombinantly modified at one or more corresponding amino acid position(s) (i.e., corresponding positions that match or span a similar aligned sequence element according to accepted alignment methods to residues marking the structural element of interest in a heterologous reference peptide sequence, such as an isoform, species or allelic variant, or synthetic mutant, of the subject proinflammatory or anti-inflammatory binding peptide) to encode an amino acid deletion, substitution, or insertion that alters corresponding residue(s) in the native peptide to generate an operable peptide analog within the invention □ having an analogous structural and/or functional element as the reference peptide.

Within this rational design method for constructing biologically active peptide analogs, the native or wild-type identity of residue(s) at amino acid positions corresponding to a structural element of interest in a heterologous reference peptide may be altered to the same, or a conservatively related, residue identity as the corresponding amino acid residue(s) in the reference peptide. However, it is often possible to alter native amino acid residues non-conservatively with respect to the corresponding reference protein residue(s). In particular, many non-conservative amino acid substitutions, particularly at divergent sites suggested to be more amenable to modification, may yield a moderate impairment or neutral effect, or even enhance a selected biological activity, compared to the function of a native peptide.

Sequence alignment and comparisons to forecast useful peptide and protein analogs and mimetics will be further refined by analysis of crystalline structure (see, e.g., Löebermann et al., J. Molec. Biol. 177:531-556, 1984; Huber et al., Biochemistry 28:8951-8966, 1989; Stein et al., Nature 347:99-102, 1990; Wei et al., Structural Biology 1:251-255, 1994, each incorporated herein by reference) of native biologically active peptides, coupled with computer modeling methods known in the art. These analyses allow detailed structure-function mapping to identify desired structural elements and modifications for incorporation into peptide and protein analogs and mimetics that will exhibit substantial activity comparable to that of the native peptide for use within the methods and compositions of the invention.

Biologically active peptide and protein analogs of the invention typically show substantial sequence identity to a corresponding native peptide sequence. The term “substantial sequence identity” means that the two subject amino acid sequences, when optimally aligned, such as by the programs GAP or BESTFIT using default gap penalties, share at least 65 percent sequence identity, commonly 80-85% percent sequence identity, often at least 90-95 percent or greater sequence identity. “Percentage amino acid identity” refers to a comparison of the amino acid sequences of two peptides which, when optimally aligned, have approximately the designated percentage of the same amino acids. Sequence comparisons are generally made to a reference sequence over a comparison window of at least 10 residue positions, frequently over a window of at least 15-20 amino acids, wherein the percentage of sequence identity is calculated by comparing a reference sequence to a second sequence, the latter of which may represent, for example, a peptide analog sequence that includes one or more deletions, substitutions or additions which total 20 percent, typically less than 5-10% of the reference sequence over the window of comparison. The reference sequence may be a subset of a larger sequence, for example, a subset of residues from a hsp60 leader sequence. Optimal alignment of sequences for aligning a comparison window may be conducted according to the local homology algorithm of Smith and Waterman (Adv. Appl. Math. 2:482, 1981), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), or by computerized implementations of these algorithms (GAP, BESTFIT, FASTA, and/or TFASTA, e.g., as provided in the Wisconsin Genetics Software Package Release 7.0, Genetics Computer Group, 575 Science Dr., Madison, Wis.).

By aligning a peptide analog optimally with a corresponding native peptide, and by using appropriate assays, e.g., adhesion protein or receptor binding assays, to determine a selected biological activity, one can readily identify operable peptide and protein analogs for use within the methods and compositions of the invention. Operable peptide and protein analogs are typically specifically immunoreactive with antibodies raised to the corresponding native peptide. Likewise, nucleic acids encoding operable peptide and protein analogs will share substantial sequence identity as described above to a nucleic acid encoding the corresponding native peptide, and will typically selectively hybridize to a partial or complete nucleic acid sequence encoding the corresponding native peptide, or fragment thereof, under accepted, moderate or high stringency hybridization conditions (see, e.g., Sambrook et al., Molecular Cloning: A Laboratory Manual, 3^{rd} Edition, Cold Spring Harbor Laboratories, Cold Spring Harbor, N.Y., 2001, incorporated herein by reference). The phrase “selectively hybridizing to” refers to a selective interaction between a nucleic acid probe that hybridizes, duplexes or binds preferentially to a particular target DNA or RNA sequence, for example when the target sequence is present in a heterogenous preparation such as total cellular DNA or RNA. Generally, nucleic acid sequences encoding biologically active peptide and protein analogs, or fragments thereof, will hybridize to nucleic acid sequences encoding the corresponding native peptide under stringent conditions (e.g., selected to be about 5° C. lower than the thermal melting point (Tm) for the subject sequence at a defined ionic strength and pH, where the Tm is the temperature under defined ionic strength and pH at which 50% of the complementary or target sequence hybridizes to a perfectly matched probe). For discussions of nucleic acid probe design and annealing conditions, see, for example, Sambrook et al., Molecular Cloning: A Laboratory Manual, 3rd Edition, Vols. 1-3, Cold Spring Harbor Laboratory, 2001 or Current Protocols in Molecular Biology, F. Ausubel et al, ed., Greene Publishing and Wiley-Interscience, New York, 1987, each of which is incorporated herein by reference. Typically, stringent or selective conditions will be those in which the salt concentration is at least about 0.02 molar at pH 7 and the temperature is at least about 60□C. Less stringent selective hybridization conditions may also be chosen. As other factors may significantly affect the stringency of hybridization, including, among others, base composition and size of the complementary strands, the presence of organic solvents and the extent of base mismatching, the combination of parameters is more important than the specific measure of any one.

Within additional aspects of the invention, peptide mimetics are provided which comprise a peptide or non-peptide molecule that mimics the tertiary binding structure and activity of a selected native peptide functional domain (e.g., binding motif or active site). These peptide mimetics include recombinantly or chemically modified peptides, as well as non-peptide agents such as small molecule drug mimetics, as further described below.

In one aspect, peptides (including polypeptides) useful within the invention are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl.

Peptides, as well as peptide and protein analogs and mimetics, can also be covalently bound to one or more of a variety of nonproteinaceous polymers, e.g., polyethylene glycol, polypropylene glycol, or polyoxyalkenes, in the manner set forth in U.S. Pat. No. 4,640,835; U.S. Pat. No. 4,496,689; U.S. Pat. No. 4,301,144; U.S. Pat. No. 4,670,417; U.S. Pat. No. 4,791,192; or U.S. Pat. No. 4,179,337, all which-are incorporated by reference in their entirety herein.

Other peptide and protein analogs and mimetics within the invention include glycosylation variants, and covalent or aggregate conjugates with other chemical moieties. Covalent derivatives can be prepared by linkage of functionalities to groups which are found in amino acid side chains or at the N- or C-termini, by means which are well known in the art. These derivatives can include, without limitation, aliphatic esters or amides of the carboxyl terminus, or of residues containing carboxyl side chains, 0-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C 18 normal alkyl, thereby forming alkanoyl aroyl species. Covalent attachment to carrier proteins, e.g., immunogenic moieties may also be employed.

In addition to these modifications, glycosylation alterations of biologically active peptides can be made, e.g., by modifying the glycosylation patterns of a peptide during its synthesis and processing, or in further processing steps. Particularly preferred means for accomplishing this are by exposing the peptide to glycosylating enzymes derived from cells that normally provide such processing, e.g., mammalian glycosylation enzymes. Deglycosylation enzymes can also be successfully employed to yield useful modified peptides within the invention. Also embraced are versions of a native primary amino acid sequence which have other minor modifications, including phosphorylated amino acid residues, e.g., phosphotyrosine, phosphoserine, or phosphothreonine, or other moieties, including ribosyl groups or cross-linking reagents.

Peptidomimetics may also have amino acid residues that have been chemically modified by phosphorylation, sulfonation, biotinylation, or the addition or removal of other moieties, particularly those that have molecular shapes similar to phosphate groups. In some embodiments, the modifications will be useful labeling reagents, or serve as purification targets, e.g., affinity ligands.

A major group of peptidomimetics within the invention comprises covalent conjugates of native peptides, or fragments thereof, with other proteins or peptides. These derivatives can be synthesized in recombinant culture such as N- or C-terminal fusions or by the use of agents known in the art for their usefulness in cross-linking proteins through reactive side groups. Preferred peptide and protein derivatization sites for targeting by cross-linking agents are at free amino groups, carbohydrate moieties, and cysteine residues.

Fusion polypeptides between biologically active peptides and other homologous or heterologous peptides are also provided. Many growth factors and cytokines are homodimeric entities, and a repeat construct of these molecules or active fragments thereof will yield various advantages, including lessened susceptibility to proteolytic degradation. Repeat and other fusion constructs of proinflammatory or anti-inflammatory binding peptide yield similar advantages within the methods and compositions of the invention. Various alternative multimeric constructs comprising peptides useful within the invention are thus provided. In certain embodiments, biologically active polypeptide fusions are provided as described in U.S. Pat. Nos. 6,018,026, 5,843,725, 6,291,646, 6,300,099, and 6,323,323 (each incorporated herein by reference), for example by linking one or more biologically active peptides of the invention with a heterologous, multimerizing polypeptide, for example an immunoglobulin heavy chain constant region, or an immunoglobulin light chain constant region. The biologically active, multimerized polypeptide fusion thus constructed can be a hetero- or homo-multimer, e.g., a heterodimer or homodimer comprising one or more proinflammatory or anti-inflammatory binding peptide element(s), which may each comprise one or more distinct biologically active peptides operable within the invention. Other heterologous polypeptides may be combined with the active peptide to yield fusions that exhibit a combination of properties or activities of the derivative proteins. Other typical examples are fusions of a reporter polypeptide, e.g., CAT or luciferase, with a peptide as described herein, to facilitate localization of the fused peptide (see, e.g., Dull et al., U.S. Pat. No. 4,859,609, incorporated herein by reference). Other fusion partners useful in this context include bacterial beta-galactosidase, trpE, Protein A, beta-lactamase, alpha amylase, alcohol dehydrogenase, and yeast alpha mating factor (see, e.g., Godowski et al., Science 241:812-816, 1988, incorporated herein by reference).

The present invention also contemplates the use of biologically active peptides modified by covalent or aggregative association with chemical moieties. These derivatives generally fall into the three classes: (1) salts, (2) side chain and terminal residue covalent modifications, and (3) adsorption complexes, for example with cell membranes. Such covalent or aggregative derivatives are useful for various purposes, for example to block homo- or heterotypic association between one or more proinflammatory or anti-inflammatory binding peptide(s), as immunogens, as reagents in immunoassays, or in purification methods such as for affinity purification of ligands or other binding ligands. For example, an active peptide can be immobilized by covalent bonding to a solid support such as cyanogen bromide-activated Sepharose, by methods which are well known in the art, or adsorbed onto polyolefin surfaces, with or without glutaraldehyde cross-linking, for use in the assay or purification of antibodies that specifically bind the active peptide. The active peptide can also be labeled with a detectable group, for example radioiodinated by the chloramine T procedure, covalently bound to rare earth chelates, or conjugated to another fluorescent moiety for use in diagnostic assays, including assays involving intranasal administration of the labeled peptide.

Those of skill in the art recognize that a variety of techniques are available for constructing peptide and protein mimetics with the same or similar desired biological activity as the corresponding native peptide but with more favorable activity than the peptide, for example improved characteristics of solubility, stability, and/or susceptibility to hydrolysis or proteolysis (see, e.g., Morgan and Gainor, Ann. Rep. Med. Chem. 24:243-252, 1989, incorporated herein by reference). Certain peptidomimetic compounds are based upon the amino acid sequence of the proteins and peptides described herein for use within the invention. Typically, peptidomimetic compounds are synthetic compounds having a three-dimensional structure (of at least part of the mimetic compound) that mimics, e.g., the primary, secondary, and/or tertiary structural, and/or electrochemical characteristics of a selected peptide, or a structural domain, active site, or binding region (e.g., a homotypic or heterotypic binding site, catalytic active site or domain, receptor or ligand binding interface or domain, etc.) thereof. The peptide-mimetic structure or partial structure (also referred to as a peptidomimetic “motif” of a peptidomimetic compound) will share a desired biological activity with a native peptide, e.g., activity to bind HLA-E or block binding of a protective HLA-E binding or recognition by a CD94/NKG2 cellular receptor of a MHC leader sequence peptide/HLA-E complex. Typically, the subject biologically activity of the mimetic compound is not substantially reduced in comparison to, and is often the same as or greater than, the activity of the native peptide on which the mimetic was modeled. In addition, peptidomimetic compounds can have other desired characteristics that enhance their therapeutic application, such as increased cell permeability, greater affinity and/or avidity, and prolonged biological half-life. The peptidomimetics of the invention will sometimes have a “backbone” that is partially or completely non-peptide, but with side groups identical to the side groups of the amino acid residues that occur in the peptide on which the peptidomimetic is modeled. Several types of chemical bonds, e.g. ester, thioester, thioamide, retroamide, reduced carbonyl, dimethylene and ketomethylene bonds, are known in the art to be generally useful substitutes for peptide bonds in the construction of protease-resistant peptidomimetics.

The following describes methods for preparing peptide and protein mimetics modified at the N-terminal amino group, the C-terminal carboxyl group, and/or changing ore or more of the amido linkages in the peptide to a non-amido linkage. It being understood that two or more such modifications can be coupled in one peptide mimetic structure (e.g., modification at the C-terminal carboxyl group and inclusion of a —CH₂-carbamate linkage between two amino acids in the peptide. For N-terminal modifications, peptides typically are synthesized as the free acid but, as noted above, can be readily prepared as the amide or ester. One can also modify the amino and/or carboxy terminus of peptide compounds to produce other compounds useful within the invention. Amino terminus modifications include methylating (i.e., —NHCH₃ or —NH(CH₃)₂), acetylating, adding a carbobenzoyl group, or blocking the amino terminus with any blocking group containing a carboxylate functionality defined by RCOO—, where R is selected from the group consisting of naphthyl, acridinyl, steroidyl, and similar groups. Carboxy terminus modifications include replacing the free acid with a carboxamide group or forming a cyclic lactam at the carboxy terminus to introduce structural constraints. Amino terminus modifications are as recited above and include alkylating, acetylating, adding a carbobenzoyl group, forming a succinimide group, etc. The N-terminal amino group can then be reacted as follows:

(a) to form an amide group of the formula RC(O)NH— where R is as defined above by reaction with an acid halide [e.g., RC(O)Cl] or acid anhydride: Typically, the reaction can be conducted by contacting about equimolar or excess amounts (e.g., about 5 equivalents) of an acid halide to the peptide in an inert diluent (e.g., dichloromethane) preferably containing an excess (e.g., about 10 equivalents) of a tertiary amine, such as diisopropylethylamine, to scavenge the acid generated during reaction. Reaction conditions are otherwise conventional (e.g., room temperature for 30 minutes). Alkylation of the terminal amino to provide for a lower alkyl N-substitution followed by reaction with an acid halide as described above will provide for N-alkyl amide group of the formula RC(O)NR—;

(b) to form a succinimide group by reaction with succinic anhydride. As before, an approximately equimolar amount or an excess of succinic anhydride (e.g., about 5 equivalents) can be employed and the amino group is converted to the succinimide by methods well known in the art including the use of an excess (e.g., ten equivalents) of a tertiary amine such as diisopropylethylamine in a suitable inert solvent (e.g., dichloromethane) (see, for example, Wollenberg, et al., U.S. Pat. No. 4,612,132, incorporated herein by reference). It is understood that the succinic group can be substituted with, for example, C₂-C₆ alkyl or —SR substituents that are prepared in a conventional manner to provide for substituted succinimide at the N-terminus of the peptide. Such alkyl substituents are prepared by reaction of a lower olefin (C₂-C₆) with maleic anhydride in the manner described by Wollenberg, et al. (U.S. Pat. No. 4,612,132) and —SR substituents are prepared by reaction of RSH with maleic anhydride where R is as defined above;

(c) to form a benzyloxycarbonyl-NH— or a substituted benzyloxycarbonyl-NH— group by reaction with approximately an equivalent amount or an excess of CBZ-Cl (i.e., benzyloxycarbonyl chloride) or a substituted CBZ-Cl in a suitable inert diluent (e.g., dichloromethane) preferably containing a tertiary amine to scavenge the acid generated during the reaction;

(d) to form a sulfonamide group by reaction with an equivalent amount or an excess (e.g., 5 equivalents) of R—S(O)₂Cl in a suitable inert diluent (dichloromethane) to convert the terminal amine into a sulfonamide where R is as defined above. Preferably, the inert diluent contains excess tertiary amine (e.g., ten equivalents) such as diisopropylethylamine, to scavenge the acid generated during reaction. Reaction conditions are otherwise conventional (e.g., room temperature for 30 minutes);

(e) to form a carbamate group by reaction with an equivalent amount or an excess (e.g., 5 equivalents) of R—OC(O)Cl or R—OC(O)OC₆H₄-p-NO₂ in a suitable inert diluent (e.g., dichloromethane) to convert the terminal amine into a carbamate where R is as defined above. Preferably, the inert diluent contains an excess (e.g., about 10 equivalents) of a tertiary amine, such as diisopropylethylamine, to scavenge any acid generated during reaction. Reaction conditions are otherwise conventional (e.g., room temperature for 30 minutes);

(f) to form a urea group by reaction with an equivalent amount or an excess (e.g., 5 equivalents) of R—N═C═O in a suitable inert diluent (e.g., dichloromethane) to convert the terminal amine into a urea (i.e., RNHC(O)NH—) group where R is as defined above. Preferably, the inert diluent contains an excess (e.g.,.about 10 equivalents) of a tertiary amine, such as diisopropylethylamine. Reaction conditions are otherwise conventional (e.g., room temperature for about 30 minutes).

In preparing peptide mimetics wherein the C-terminal carboxyl group is replaced by an ester (i.e., —C(O)OR where R is as defined above), resins as used to prepare peptide acids are typically employed, and the side chain protected peptide is cleaved with base and the appropriate alcohol, e.g., methanol. Side chain protecting groups are then removed in the usual fashion by treatment with hydrogen fluoride to obtain the desired ester.

In preparing peptide mimetics wherein the C-terminal carboxyl group is replaced by the amide —C(O)NR₃R₄, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH₂). Alternatively, use of the chloromethylated resin during peptide synthesis coupled with reaction with ammonia to cleave the side chain protected peptide from the support yields the free peptide amide and reaction with an alkylamine or a dialkylamine yields a side chain protected alkylamide or dialkylamide (i.e., the C-terminus is —C(O)NRR, where R and R¹ are as defined above). Side chain protection is then removed in the usual fashion by treatment with hydrogen fluoride to give the free amides, alkylamides, or dialkylamides.

In another alternative embodiments of the invention, the C-terminal carboxyl group or a C-terminal ester of a biologically active peptide can be induced to cyclize by internal displacement of the —OH or the ester (—OR) of the carboxyl group or ester respectively with the N-terminal amino group to form a cyclic peptide., For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Such methods are well known in the art.

One can cyclize active peptides for use within the invention, or incorporate a desamino or descarboxy residue at the termini of the peptide, so that there is no terminal amino or carboxyl group, to decrease susceptibility to proteases, or to restrict the conformation of the peptide. C-terminal functional groups among peptide analogs and mimetics of the present invention include amide, amide lower alkyl, amide di(lower alkyl), lower alkoxy, hydroxy, and carboxy, and the lower ester derivatives thereof, and the pharmaceutically acceptable salts thereof.

Other methods for making peptide and protein derivatives and mimetics for use within the methods and compositions of the invention are described in Hruby et al. (Biochem J. 268(2):249-262, 1990, incorporated herein by reference). According to these methods, biologically active peptides serve as structural models for non-peptide mimetic compounds having similar biological activity as the native peptide. Those of skill in the art recognize that a variety of techniques are available for constructing compounds with the same or similar desired biological activity as the lead peptide compound, or that have more favorable activity than the lead with respect a desired property such as solubility, stability, and susceptibility to hydrolysis and proteolysis (see, e.g., Morgan and Gainor, Ann. Rep. Med. Chem. 24:243-252, 1989, incorporated herein by reference). These techniques include, for example, replacing a peptide backbone with a backbone composed of phosphonates, amidates, carbamates, sulfonamides, secondary amines, and/or N-methylamino acids.

Peptide and protein mimetics wherein one or more of the peptidyl linkages [—C(O)NH—] have been replaced by such linkages as a —CH₂-carbamate linkage, a phosphonate linkage, a —CH₂-sulfonamide linkage, a urea linkage, a secondary amine (—CH₂NH—) linkage, and an alkylated peptidyl linkage [—C(O)NR₆— where R6 is lower alkyl] are prepared, for example, during conventional peptide synthesis by merely substituting a suitably protected amino acid analogue for the amino acid reagent at the appropriate point during synthesis. Suitable reagents include, for example, amino acid analogues wherein the carboxyl group of the amino acid has been replaced with a moiety suitable for forming one of the above linkages. For example, if one desires to replace a —C(O)NR— linkage in the peptide with a —CH₂-carbamate linkage (—CH₂OC(O)NR—), then the carboxyl (—COOH) group of a suitably protected amino acid is first reduced to the —CH₂OH group which is then converted by conventional methods to a —OC(O)Cl functionality or a para-nitrocarbonate —OC(O)O—C₆H₄-p-NO₂ functionality. Reaction of either of such functional groups with the free amine or an alkylated amine on the N-terminus of the partially fabricated peptide found on the solid support leads to the formation of a —CH₂OC(O)NR— linkage. For a more detailed description of the formation of such —CH₂-carbamate linkages, see, e.g., Cho et al. (Science 261:1303-1305, 1993, incorporated herein by reference).

Replacement of an amido linkage in an active peptide with a —CH₂-sulfonamide linkage can be achieved by reducing the carboxyl (—COOH) group of a suitably protected amino acid to the —CH₂OH group, and the hydroxyl group is then converted to a suitable leaving group such as a tosyl group by conventional methods. Reaction of the derivative with, for example, thioacetic acid followed by hydrolysis and oxidative chlorination will provide for the —CH₂—S(O)₂Cl functional group which replaces the carboxyl group of the otherwise suitably protected amino acid. Use of this suitably protected amino acid analogue in peptide synthesis provides for inclusion of an —CH₂S(O)₂NR— linkage that replaces the amido linkage in the peptide thereby providing a peptide mimetic. For a more complete description on the conversion of the carboxyl group of the amino acid to a —CH₂S(O)₂Cl group, see, e.g., Weinstein and Boris (Chemistry & Biochemistry of Amino Acids, Peptides, Vol. 7, pp. 267-357, Marcel Dekker, Inc., New York, 1983, incorporated herein by reference). Replacement of an amido linkage in an active peptide with a urea linkage can be achieved, for example, in the manner set forth in U.S. patent application Ser. No. 08/147,805 (incorporated herein by reference).

Secondary amine linkages wherein a —CH₂NH— linkage replaces the amido linkage in the peptide can be prepared by employing, for example, a suitably protected dipeptide analogue wherein the carbonyl bond of the amido linkage has been reduced to a CH₂ group by conventional methods. For example, in the case of diglycine, reduction of the amide to the amine will yield after deprotection H₂NCH₂CH₂NHCH₂ COOH that is then used in N-protected form in the next coupling reaction. The preparation of such analogues by reduction of the carbonyl group of the amido linkage in the dipeptide is well known in the art.

The biologically active peptide and protein agents of the present invention may exist in a monomeric form with no disulfide bond formed with the thiol groups of cysteine residue(s) that may be present in the subject peptide. Alternatively, an intermolecular disulfide bond between thiol groups of cysteines on two or more peptides can be produced to yield a multimeric (e.g., dimeric, tetrameric or higher oligomeric) compound. Certain of such peptides can be cyclized or dimerized via displacement of the leaving group by the sulfur of a cysteine or homocysteine residue (see, e.g., Barker et al., J. Med. Chem. 35:2040-2048, 1992; and Or et al., J. Org. Chem. 56:3146-3149, 1991, each incorporated herein by reference). Thus, one or more native cysteine residues may be substituted with a homocysteine. Intramolecular or intermolecular disulfide derivatives of active peptides provide analogs in which one of the sulfurs has been replaced by a CH2 group or other isostere for sulfur. These analogs can be made via an intramolecular or intermolecular displacement, using methods known in the art.

All of the naturally occurring, recombinant, and synthetic peptides, and the peptide and protein analogs and mimetics, identified as useful agents within the invention can be used for screening (e.g., in kits and/or screening assay methods) to identify additional compounds, including other peptides, proteins, analogs and mimetics, that will function within the methods and compositions of the invention, including as inhibitors of homotypic and heterotypic binding between membrane adhesive proteins to enhance epithelial permeability. Several methods of automating assays have been developed in recent years so as to permit screening of tens of thousands of compounds in a short period (see, e.g., Fodor et al., Science 251:767-773, 1991, and U.S. Pat. Nos. 5,677,195; 5,885,837; 5,902,723; 6,027,880; 6,040,193; and 6,124,102, issued to Fodor et al., each incorporated herein by reference). Large combinatorial libraries of compounds can be constructed by encoded synthetic libraries (ESL) described in, e.g., WO 95/12608, WO 93/06121, WO 94/08051, WO 95/35503, and WO 95/30642 (each incorporated by reference). Peptide libraries can also be generated by phage display methods (see, e.g., Devlin, WO 91/18980, incorporated herein by reference). Many other publications describing chemical diversity libraries and screening methods are also considered reflective of the state of the art pertaining to these aspects of the invention and are generally incorporated herein.

One method of screening for new biologically active agents for use within the invention (e.g., small molecule drug peptide mimetics) utilizes eukaryotic or prokaryotic host cells which are stably transformed with recombinant DNA molecules expressing an active peptide. Such cells, either in viable or fixed form, can be used for standard assays, e.g., ligand/receptor binding assays (see, e.g., Parce et al., Science 246:243-247, 1989; and Owicki et al., Proc. Natl. Acad. Sci. USA 87:4007-4011, 1990, each incorporated herein by reference). Competitive assays are particularly useful, for example assays where the cells are contacted and incubated with a labeled receptor or antibody having known binding affinity to the peptide ligand, and a test compound or sample whose binding affinity is being measured. The bound and free labeled binding components are then separated to assess the degree of ligand binding. The amount of test compound bound is inversely proportional to the amount of labeled receptor binding to the known source. Any one of numerous techniques can be used to separate bound from free ligand to assess the degree of ligand binding. This separation step can involve a conventional procedure such as adhesion to filters followed by washing, adhesion to plastic followed by washing, or centrifugation of the cell membranes.

Another technique for drug screening within the invention involves an approach which provides high throughput screening for compounds having suitable binding affinity to a target molecule, e.g., a HLA-E molecule, HLA-E peptide complex, or HLA-E/peptide/CD94/NKG2 cellular receptor complex, and is described in detail in Geysen, European Patent Application 84/03564, published on Sep. 13, 1984 (incorporated herein by reference). First, large numbers of different test compounds, e.g., small peptides, are synthesized on a solid substrate, e.g., plastic pins or some other appropriate surface, (see, e.g., Fodor et al., Science 251:767-773, 1991, and U.S. Pat. Nos. 5,677,195; 5,885,837; 5,902,723; 6,027,880; 6,040,193; and 6,124,102, issued to Fodor et al., each incorporated herein by reference). Then all of the pins are reacted with a solubilized peptide agent of the invention, and washed. The next step involves detecting bound peptide.

Rational drug design may also be based upon structural studies of the molecular shapes of biologically active peptides determined to operate within the methods of the invention. Various methods are available and well known in the art for characterizing, mapping, translating, and reproducing structural features of peptides to guide the production and selection of new peptide mimetics, including for example x-ray crystallography and 2 dimensional NMR techniques. These and other methods, for example, will allow reasoned prediction of which amino acid residues present in a selected peptide form molecular contact regions necessary for specificity and activity (see, e.g., Blundell and Johnson, Protein Crystallography, Academic Press, N.Y., 1976, incorporated herein by reference).

Operable analogs and mimetics of proinflammatory or anti-inflammatory binding peptides disclosed herein retain partial, complete or enhanced activity compared to a native peptide. In this regard, operable analogs and mimetics for use within the invention will retain at least 50%, often 75%, and up to 95-100% or greater levels of one or more selected activities as compared to the same activity observed for a selected native peptide or unmodified compound. These biological properties of altered peptides or non-peptide mimetics can be determined according to any suitable assay disclosed or incorporated herein.

In accordance with the description herein, the compounds of the invention are useful in vitro as unique tools for analyzing the nature and function of proinflammatory or anti-inflammatory binding peptides, HLA-E molecules, and CD94/NKG2 cellular receptors, and will therefore also serve as leads in various programs for designing additional peptide and non-peptide (e.g., small molecule drug) agents for enhancing mucosal epithelial permeability and facilitating mucosal drug delivery.

In addition, the proinflammatory or anti-inflammatory binding peptides, analogs and mimetics disclosed herein are useful as immunogens, or components of immunogens, for generating antibodies and related agents that will be useful, for example, to block HLA-E binding by a proinflammatory binding peptide to alleviate symptoms of autoimmunity or inflammation, or to target or trigger NK and CTL responses against tumor cells or virally infected cells. In the latter context, localization of the antibody to the tumor or viral infected cell or tissue may be facilitated by coupling of the antibody to a tumor or viral targeting factor, e.g. an antibody or antibody fragment that binds a tumor-associated or viral-associated antigen.

Thus, the peptides of the invention will be administered as immunogens, typically in the form of a conjugate (e.g., a multimeric peptide, or a peptide/carrier or peptide/hapten conjugate), to generate antibodies that bind the immunizing peptide(s) or peptide conjugate(s) with high affinity or avidity, but do not similarly recognize unrelated peptides.

In this context, the invention also provides diagnostic and therapeutic antibodies, including monoclonal antibodies, directed against a proinflammatory or anti-inflammatory binding peptide. The antibodies may specifically recognize functional portions of the peptide involved in interactions between the peptide and, e.g., an HLA-E molecule. These immunotherapeutic reagents may include humanized antibodies, and can be combined for therapeutic use with additional active or inert ingredients as disclosed herein, e.g., in conventional pharmaceutically acceptable carriers or diluents, e.g., immunogenic adjuvants, and optionally with adjunctive or combinatorially active agents such as antiretroviral drugs. Methods for generating functional antibodies, including humanized antibodies, antibody fragments, and other related agents are well known in the art (see, e.g., Harlow & Lane, Antibodies, A Laboratory Manual, CSHP, NY, 1988; Queen et al., Proc. Natl. Acad. Sci. USA 86:10029-10033, 1989 and WO 90/07861, each incorporated by reference).

Humanized forms of mouse antibodies can be generated by linking the CDR regions of non-human antibodies to human constant regions by recombinant DNA techniques (see, e.g., Queen et al., Proc. Natl. Acad. Sci. USA 86:10029-10033, 1989 and WO 90/07861, each incorporated by reference). Human antibodies can be obtained using phage-display methods (see, e.g., Dower et al., WO 91/17271; McCafferty et al., WO 92/01047, each incorporated herein by reference). In these methods, libraries of phage are produced in which members display different antibodies on their outersurfaces. Antibodies are usually displayed as Fv or Fab fragments. Phage displaying antibodies with a desired specificity are selected by affinity enrichment to human cytochrome P450 or a fragment thereof. Human antibodies are selected by competitive binding experiments, or otherwise, to have the same epitope specificity as a particular mouse antibody.

The invention further provides fragments of the intact antibodies described above. Typically, these fragments compete with the intact antibody from which they were derived for specific binding to HLA. Antibody fragments include separate heavy chains, light chains Fab, Fab′ F(ab′)2, Fv, and single chain antibodies. Fragments can be produced by enzymic or chemical separation of intact immunoglobulins. For example, a F(ab′)2 fragment can be obtained from an IgG molecule by proteolytic digestion with pepsin at pH 3.0-3.5 using standard methods such as those described in Harlow and Lane, supra. Fab fragments may be obtained from F(ab′)2 fragments by limited reduction, or from whole antibody by digestion with papain in the presence of reducing agents. Fragments can also be produced by recombinant DNA techniques. Segments of nucleic acids encoding selected fragments are produced by digestion of full-length coding sequences with restriction enzymes, or by de novo synthesis. Often fragments are expressed in the form of phage-coat fusion proteins. This manner of expression is advantageous for affinity-sharpening of antibodies.

To produce antibodies of the invention recombinantly, nucleic acids encoding light and heavy chain variable regions, optionally linked to constant regions, are inserted into expression vectors. The light and heavy chains can be cloned in the same or different expression vectors. The DNA segments encoding antibody chains are operably linked to control sequences in the expression vector(s) that ensure the expression of antibody chains. Such control sequences include a signal sequence, a promoter, an enhancer, and a transcription termination sequence. Expression vectors are typically replicable in the host organisms either as episomes or as an integral part of the host chromosome. E. coli is one procaryotic host particularly useful for expressing antibodies of the present invention. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which typically contain expression control sequences compatible with the host cell (e.g., an origin of replication) and regulatory sequences such as a lactose promoter system, a tryptophan (trp) promoter system, a beta-lactamase promoter system, or a promoter system from phage lambda. Other microbes, such as yeast, may also be used for expression. Saccharomyces is a preferred host, with suitable vectors having expression control sequences, such as promoters, including 3-phosphoglycerate kinase or other glycolytic enzymes, and an origin of replication, termination sequences and the like as desired.

Mammalian tissue cell culture can also be used to express and produce the antibodies of the present invention (see, e.g., Winnacker, From Genes to Clones, VCH Publishers, N.Y., 1987, incorporated herein by reference). Eukaryotic cells are preferred, because a number of suitable host cell lines capable of secreting intact antibodies have been developed. Preferred suitable host cells for expressing nucleic acids encoding the immunoglobulins of the invention include: monkey kidney CV1 line transformed by SV40 (COS-7, ATCC CRL 1651); human embryonic kidney line (293) (Graham et al., J. Gen. Virol. 36:59, 1977, incorporated herein by reference); baby hamster kidney cells (BHK, ATCC CCL 10); Chinese hamster ovary-cells-DHFR (CHO, Urlaub and Chasin, Proc. Natl. Acad. Sci. USA 77:4216, 1980, incorporated herein by reference); mouse sertoli cells (TM4, Mather, Biol. Reprod. 23:243-251, 1980, incorporated herein by reference); monkey kidney cells (CV1 ATCC CCL 70); african green monkey kidney cells (VERO-76, ATCC CRL 1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); and, TRI cells (Mather et al., Annals N.Y. Acad. Sci. 383:4446, 1982, incorporated herein by reference); and baculovirus cells.

The vectors containing the polynucleotide sequences of interest (e.g., the heavy and light chain encoding sequences and expression control sequences) can be transferred into the host cell. Calcium chloride transfection is commonly utilized for prokaryotic cells, whereas calcium phosphate treatment or electroporation can be used for other cellular hosts (see, e.g., Sambrook et al., Molecular Cloning: A Laboratory Manual, Cold Spring Harbor Press, 2nd ed., 1989, incorporated herein by reference). When heavy and light chains are cloned on separate expression vectors, the vectors are co-transfected to obtain expression and assembly of intact immunoglobulins. After introduction of recombinant DNA, cell lines expressing immunoglobulin products are cell selected. Cell lines capable of stable expression are preferred (i.e., undiminished levels of expression after fifty passages of the cell line).

Once expressed, the whole antibodies, their dimers, individual light and heavy chains, or other immunoglobulin forms of the present invention can be purified according to standard procedures of the art, including ammonium sulfate precipitation, affinity columns, column chromatography, gel electrophoresis and the like (see, e.g., Scopes, Protein Purification, Springer-Verlag, N.Y., 1982, incorporated herein by reference). Substantially pure immunoglobulins of at least about 90 to 95% homogeneity are preferred, and 98 to 99% or more homogeneity most preferred.

The proinflammatory or anti-inflammatory binding peptides of the invention can also generally be used in drug screening compositions and procedures, as noted above, e.g., to identify additional compounds having binding affinity to HLA-E, HLA-E/peptide complexes, or HLA-E/peptide/CD94/NKG2 cellular receptor complexes and/or act as agonists or antagonists to HLA-E mediated protective interactions with CD94/NKG2 cellular receptors, and thereby function as immune modulatory agents as described herein. Various screening methods and formats are available and well known in the art. Subsequent biological assays can then be utilized to determine if the screened compound has intrinsic binding or other desired activity useful within the invention. In such assays, the compounds of the invention can be used without modification or can be modified in a variety of ways; for example, by labeling, such as covalently or non-covalently joining a moiety which directly or indirectly provides a detectable signal. Possibilities for direct labeling include label groups such as: radiolabels, enzymes such as peroxidase and alkaline phosphatase (see, e.g., U.S. Pat. No. 3,645,090; and U.S. Pat. No. 3,940,475, each incorporated herein by reference), and fluorescent labels. Possibilities for indirect labeling include biotinylation of one constituent followed by binding to avidin coupled to one of the above label groups. The compounds may also include spacers or linkers in cases where the compounds are to be attached to a solid support.

The proinflammatory or anti-inflammatory binding peptides of the invention can also be employed, based on their ability to bind HLA-E and complexes with CD94/NKG2 cellular receptor, as reagents for detecting and/or quantifying HLA-E molecules on living cells, fixed cells, in biological fluids, in tissue homogenates, in purified, natural biological materials, etc. For example, by labeling such peptides, one can identify and/or quantify cells having HLA-E molecules on their surfaces. In addition, based on their more detailed activities, the proinflammatory or anti-inflammatory binding peptides can be used to quantify the presence and activity of other HLA-E binding peptides and CD94/NKG2 cellular receptors. The peptides of the present invention can be used in in situ staining, FACS (fluorescence-activated cell sorting), Western blotting, ELISA, etc. Further, the peptides of the present invention can be used in HLA-E and CD94/NKG2 cellular receptor purification, or in purifying cells expressing HLA-E.

The proinflammatory or anti-inflammatory binding peptides of the present invention can also be utilized as commercial reagents for various medical research and diagnostic uses. Such uses include but are not limited to: (1) use as a calibration standard for quantitating the presence or activity of HLA-E, other HLA-E binding peptides, and/or CD94/NKG2 cellular receptors; (2) use in structural analysis of HLA-E and CD94/NKG2 cellular receptor through co-crystallization; and (3) use to investigate the mechanism of HLA-E/peptide/CD94/NKG2 cellular binding and activation.

Within additional aspects of the invention, the immune modulatory activity of the subject peptides can be enhanced by linkage to a sequence which contains at least one epitope that is capable of inducing a NK, CTL, or T helper cell response. For example, a conjugate in this context may may define a proinflammatory binding peptide and one or more, different or overlapping, CTL epitopes. Alternatively, such combinatorially active peptides/epitopes can be combined in a “cocktail” to provide enhanced immunogenicity for NK or CTL responses. Peptides can also be combined with peptides having different MHC restriction elements. These compositions can be used to effectively broaden the immunological coverage provided by therapeutic, vaccine or diagnostic methods and compositions of the invention among a diverse population.

The peptides of the invention can be combined via linkage to form polymers (multimers), or can be formulated in a composition without linkage, as an admixture. Where the same peptide is linked to itself, thereby forming a homopolymer with a plurality of repeating epitopic units. Linkages for homo- or hetero-polymers or for coupling to carriers can be provided in a variety of ways. For example, cysteine residues can be added at both the amino- and carboxy-termini, where the peptides are covalently bonded via controlled oxidation of the cystein residues. Also useful are a large number of heterobifunctional agents which generate a disulfide link at one functional group end and a peptide link at the other, including N-succidimidyl-3-(2-pyridyldithio) proprionate (SPDP). This reagent creates a disulfide linkage between itself and a cysteine residue in one protein and an amide linkage through the amino on a lysine or other free amino group in the other. A variety of such disulfide/amide forming agents are known. See, for example, Immun. Rev. 62:185 (1982). Other bifunctional coupling agents form a thioether rather than a disulfide linkage. Many of these thioether-forming agents are commercially available and include reactive esters of 6-maleimidocaproic acid, 2 bromoacetic acid, 2-iodoacetic acid, 4-(N-maleimido-methyl)cyclohexane-1-carboxylic acid and the like. The carboxyl groups can be activated by combining them with succinimide or 1-hydroxy-2-nitro4-sulfonic acid, sodium salt. A particularly preferred coupling agent is succinimidyl 4-(N-maleimidomethyl)cyclohexane-1-carboxylate (SMCC). Of course, it will be understood that linkage should not substantially interfere with either of the linked groups functions.

In preferred embodiments the proinflammatory or anti-inflammatory binding peptides of the invention are conjugated to other peptides by a spacer molecule. The spacer is typically comprised of relatively small, neutral molecules, such as amino acids or amino acid mimetics, which are substantially uncharged under physiological conditions and may have linear or branched side chains. The spacers are typically selected from, e.g., Ala, Gly, or other neutral spacers of nonpolar amino acids or neutral polar amino acids. In certain preferred embodiments herein the neutral spacer is Ala. It will be understood that the optionally present spacer need not be comprised of the same residues and thus may be a hetero- or homo-oligomer. Preferred exemplary spacers are homo-oligomers of Ala. When present, the spacer will usually be at least one or two residues, more usually three to six residues.

Delivery Vehicles and Methods

Within certain aspects of the invention, proinflammatory or anti-inflammatory binding peptides are administered in a formulation that includes a biocompatible polymer functioning as a carrier or base. Such polymer carriers include polymeric powders, matrices or microparticulate delivery vehicles, among other polymer forms. The polymer can be of plant, animal, or synthetic origin. Often the polymer is crosslinked. Additionally, in these delivery systems the peptide can be functionalized in a manner where it can be covalently bound to the polymer and rendered inseparable from the polymer by simple washing. In other embodiments, the polymer is chemically modified with an inhibitor of enzymes or other agents that may degrade or inactivate the biologically active agent(s) and/or delivery enhancing agent(s).

Drug delivery systems based on biodegradable polymers are preferred in many biomedical applications because such systems are broken down either by hydrolysis or by enzymatic reaction into non-toxic molecules. The rate of degradation is controlled by manipulating the composition of the biodegradable polymer-matrix. These types of systems can therefore be employed in certain settings for long-term release of biologically active agents. Biodegradable polymers such as poly(glycolic acid) (PGA), poly-(lactic acid) (PLA), and poly(D,L-lactic-co-glycolic acid) (PLGA), have received considerable attention as possible drug delivery carriers, since the degradation products of these polymers have been found to have low toxicity. During the normal metabolic function of the body these polymers degrade into carbon dioxide and water (Mehta et al, J. Control. Rel. 29:375-384, 1994). These polymers have also exhibited excellent biocompatibility.

For prolonging the biological activity of proinflammatory or anti-inflammatory binding peptides, they may be incorporated into polymeric matrices, e.g., polyorthoesters, polyanhydrides, or polyesters. This yields sustained activity and release of the active agent(s), e.g., as determined by the degradation of the polymer matrix (Heller, Formulation and Delivery of Proteins and Peptides, pp. 292-305, Cleland et al., Eds., ACS Symposium Series 567, Washington D.C., 1994; Tabata et al., Pharm. Res. 10:487-496, 1993; and Cohen et al., Pharm. Res. 8:713-720, 1991, each incorporated herein by reference). Although the encapsulation of biotherapeutic molecules inside synthetic polymers may stabilize them during storage and delivery, the largest obstacle of polymer-based release technology is the activity loss of the therapeutic molecules during the formulation processes that often involve heat, sonication or organic solvents (Tabata et al., Pharm. Res. 10:487-496, 1993; and Jones et al., Drug Targeting and Delivery Series, New Delivery Systems for Recombinant Proteins—Practical Issues from Proof of Concept to Clinic, Vol. 4, pp. 57-67, Lee et al., Eds., Harwood Academic Publishers, 1995).

Suitable polymers for use within the invention should generally be stable alone and in combination with the selected biologically active agent(s) and additional components of a mucosal formulation, and form stable hydrogels in a range of pH conditions from about pH 1 to pH 10. More typically, they should be stable and form polymers under pH conditions ranging from about 3 to 9, without additional protective coatings. However, desired stability properties may be adapted to physiological parameters characteristic of the targeted site of delivery (e.g., nasal mucosa or secondary site of delivery such as the systemic circulation). Therefore, in certain formulations higher or lower stabilities at a particular pH and in a selected chemical or biological environment will be more desirable.

Absorption-promoting polymers of the invention may include polymers from the group of homo- and copolymers based on various combinations of the following vinyl monomers: acrylic and methacrylic acids, acrylamide, methacrylamide, hydroxyethylacrylate or methacrylate, vinylpyrrolidones, as well as polyvinylalcohol and its co- and terpolymers, polyvinylacetate, its co- and terpolymers with the above listed monomers and 2-acrylamido-2-methyl-propanesulfonic acid (AMPS®). Very useful are copolymers of the above listed monomers with copolymerizable functional monomers such as acryl or methacryl amide acrylate or methacrylate esters where the ester groups are derived from straight or branched chain alkyl, aryl having up to four aromatic rings which may contain alkyl substituents of 1 to 6 carbons; steroidal, sulfates, phosphates or cationic monomers such as N,N-dimethylaminoalkyl(meth)acrylamide, dimethylaminoalkyl(meth)acrylate, (meth)acryloxyalkyltrimethylammonium chloride, (meth)acryloxyalkyldimethylbenzyl ammonium chloride.

Additional absorption-promoting polymers for use within the invention are those classified as dextrans, dextrins, and from the class of materials classified as natural gums and resins, or from the class of natural polymers such as processed collagen, chitin, chitosan, pullalan, zooglan, alginates and modified alginates such as “Kelcoloid” (a polypropylene glycol modified alginate) gellan gums such as “Kelocogel”, Xanathan gums such as “Keltrol”, estastin, alpha hydroxy butyrate and its copolymers, hyaluronic acid and its derivatives, polylactic and glycolic acids.

A very useful class of polymers applicable within the instant invention are olefinically-unsaturated carboxylic acids containing at least one activated carbon-to-carbon olefinic double bond, and at least one carboxyl group; that is, an acid or functional group readily converted to an acid containing an olefinic double bond which readily functions in polymerization because of its presence in the monomer molecule, either in the alpha-beta position with respect to a carboxyl group, or as part of a terminal methylene grouping. Olefinically-unsaturated acids of this class include such materials as the acrylic acids typified by the acrylic acid itself, alpha-cyano acrylic acid, beta methylacrylic acid (crotonic acid), alpha-phenyl acrylic acid, beta-acryloxy propionic acid, cinnamic acid, p-chloro cinnamic acid, 1-carboxy4-phenyl butadiene-1,3, itaconic acid, citraconic acid, mesaconic acid, glutaconic acid, aconitic acid, maleic acid, fumaric acid, and tricarboxy ethylene. As used herein, the term “carboxylic acid” includes the polycarboxylic acids and those acid anhydrides, such as maleic anhydride, wherein the anhydride group is formed by the elimination of one molecule of water from two carboxyl groups located on the same carboxylic acid molecule.

Representative acrylates useful as absorption-promoting agents within the invention include methyl acrylate, ethyl acrylate, propyl acrylate, isopropyl acrylate, butyl acrylate, isobutyl acrylate, methyl methacrylate, methyl ethacrylate, ethyl methacrylate, octyl acrylate, heptyl acrylate, octyl methacrylate, isopropyl methacrylate, 2-ethylhexyl methacrylate, nonyl acrylate, hexyl acrylate, n-hexyl methacrylate, and the like. Higher alkyl acrylic esters are decyl acrylate, isodecyl methacrylate, lauryl acrylate, stearyl acrylate, behenyl acrylate and melissyl acrylate and methacrylate versions thereof. Mixtures of two or three or more long chain acrylic esters may be successfully polymerized with one of the carboxylic monomers. Other comonomers include olefins, including alpha olefins, vinyl ethers, vinyl esters, and mixtures thereof.

Yet additional useful absorption promoting materials are alpha-olefins containing from 2 to 18 carbon atoms, more preferably from 2 to 8 carbon atoms; dienes containing from 4 to 10 carbon atoms; vinyl esters and allyl esters such as vinyl acetate; vinyl aromatics such as styrene, methyl styrene and chloro-styrene; vinyl and allyl ethers and ketones such as vinyl methyl ether and methyl vinyl ketone; chloroacrylates; cyanoalkyl acrylates such as alpha-cyanomethyl acrylate, and the alpha-, beta-, and gamma-cyanopropyl acrylates; alkoxyacrylates such as methoxy ethyl acrylate; haloacrylates as chloroethyl acrylate; vinyl halides and vinyl chloride, vinylidene chloride and the like; divinyls, diacrylates and other polyfunctional monomers such as divinyl ether, diethylene glycol diacrylate, ethylene glycol dimethacrylate, methylene-bis-acrylamide, allylpentaerythritol, and the like; and bis(beta-haloalkyl) alkenyl phosphonates such as bis(beta-chloroethyl) vinyl phosphonate and the like as are known to those skilled in the art. Copolymers wherein the carboxy containing monomer is a minor constituent, and the other vinylidene monomers present as major components are readily prepared in accordance with the methods disclosed herein.

In a further related aspect, a multiligand conjugated peptide complex is provided which comprises a proinflammatory or anti-inflammatory binding peptide covalently coupled with a triglyceride backbone moiety through a polyalkylene glycol spacer group bonded at a carbon atom of the triglyceride backbone moiety, and at least one fatty acid moiety covalently attached either directly to a carbon atom of the triglyceride backbone moiety or covalently joined through a polyalkylene glycol spacer moiety (see, e.g., U.S. Pat. No. 5,681,811, incorporated herein by reference). In such a multiligand conjugated therapeutic agent complex, the alpha′ and beta carbon atoms of the triglyceride bioactive moiety may have fatty acid moieties attached by covalently bonding either directly thereto, or indirectly covalently bonded thereto through polyalkylene glycol spacer moieties. Alternatively, a fatty acid moiety may be covalently attached either directly or through a polyalkylene glycol spacer moiety to the alpha and alpha′ carbons of the triglyceride backbone moiety, with the bioactive therapeutic agent being covalently coupled with the gamma-carbon of the triglyceride backbone moiety, either being directly covalently bonded thereto or indirectly bonded thereto through a polyalkylene spacer moiety. It will be recognized that a wide variety of structural, compositional, and conformational forms are possible for the multiligand conjugated therapeutic agent complex comprising the triglyceride backbone moiety, within the scope of the invention. It is further noted that in such a multiligand conjugated therapeutic agent complex, the biologically active agent(s) may advantageously be covalently coupled with the triglyceride modified backbone moiety through alkyl spacer groups, or alternatively other acceptable spacer groups, within the scope of the invention. As used in such context, acceptability of the spacer group refers to steric, compositional, and end use application specific acceptability characteristics.

In yet additional aspects of the invention, a conjugation-stabilized complex is provided which comprises a polysorbate complex comprising a polysorbate moiety including a triglyceride backbone having covalently coupled to alpha, alpha′and beta carbon atoms thereof functionalizing groups including (i) a fatty acid group; and (ii) a polyethylene glycol group having a proinflammatory or anti-inflammatory binding peptide covalently bonded thereto, e.g., bonded to an appropriate functionality of the polyethylene glycol group (see, e.g., U.S. Pat. No. 5,681,811, incorporated herein by reference). Such covalent bonding may be either direct, e.g., to a hydroxy terminal functionality of the polyethylene glycol group, or alternatively, the covalent bonding may be indirect, e.g., by reactively capping the hydroxy terminus of the polyethylene glycol group with a terminal carboxy functionality spacer group, so that the resulting capped polyethylene glycol group has a terminal carboxy functionality to which the proinflammatory or anti-inflammatory binding peptide may be covalently bonded.

Liposomes and Micellar Delivery Vehicles

The coordinate administration methods and combinatorial formulations of the instant invention optionally incorporate effective lipid or fatty acid based carriers, processing agents, or delivery vehicles, to provide improved formulations for delivery of proinflammatory or anti-inflammatory binding peptides. For example, a variety of formulations and methods are provided for mucosal delivery which comprise one or more proinflammatory or anti-inflammatory binding peptidesadmixed or encapsulated by, or coordinately administered with, a liposome, mixed micellar carrier, or emulsion, to enhance chemical and physical stability and increase the half life of the biologically active agents (e.g., by reducing susceptibility to proteolysis, chemical modification and/or denaturation) upon mucosal delivery.

Within certain aspects of the invention, specialized delivery systems for proinflammatory or anti-inflammatory binding peptides comprise small lipid vesicles known as liposomes (see, e.g., Chonn et al., Curr. Opin. Biotechnol. 6:698-708, 1995; Lasic, Trends Biotechnol. 16:307-321, 1998; and Gregoriadis, Trends Biotechnol. 13:527-537, 1995, each incorporated herein by reference). These are typically made from natural, biodegradable, non-toxic, and non-immunogenic lipid molecules, and can efficiently entrap or bind drug molecules, including peptides and proteins, into, or onto, their membranes. The attractiveness of liposomes as a peptide and protein delivery system within the invention is increased by the fact that the encapsulated proteins can remain in their preferred aqueous environment within the vesicles, while the liposomal membrane protects them against proteolysis and other destabilizing factors. Even though not all liposome preparation methods known are feasible in the encapsulation of peptides and proteins due to their unique physical and chemical properties, several methods allow the encapsulation of these macromolecules without substantial deactivation (see, e.g., Weiner, Immunomethods 4:201-209, 1994, incorporated herein by reference).

A variety of methods are available for preparing liposomes for use within the invention (e.g., as described in Szoka et al., Ann. Rev. Biophys. Bioeng. 9:467, 1980; and U.S. Pat. Nos. 4,235,871, 4,501,728, and 4,837,028, each incorporated herein by reference). For use with liposome delivery, the biologically active agent is typically entrapped within the liposome, or lipid vesicle, or is bound to the outside of the vesicle. Several strategies have been devised to increase the effectiveness of liposome-mediated delivery by targeting liposomes to specific tissues and specific cell types. Liposome formulations, including those containing a cationic lipid, have been shown to be safe and well tolerated in human patients (Treat et al., J. Natl. Cancer Instit. 82:1706-1710, 1990, incorporated herein by reference).

Like liposomes, unsaturated long chain fatty acids, which also have enhancing activity for mucosal absorption, can form closed vesicles with bilayer-like structures (so called “ufasomes”). These can be formed, for example, using oleic acid to entrap biologically active peptides and proteins for mucosal, e.g., intranasal, delivery within the invention.

Other delivery systems for use within the invention combine the use of polymers and liposomes to ally the advantageous properties of both vehicles. Exemplifying this type of hybrid delivery system, liposomes containing the model protein horseradish peroxidase (HRP) have been effectively encapsulated inside the natural polymer fibrin (Henschen et al., Blood Coagulation, pp. 171-241, Zwaal, et al., Eds., Elsevier, Amsterdam, 1986, incorporated herein by reference). Because of its biocompatibility and biodegradability, fibrin is a useful polymer matrix for drug delivery systems in this context (see, e.g., Senderoff, et al., J. Parenter. Sci. Technol. 45:2-6, 1991; and Jackson, Nat. Med.2:637-638, 1996, incorporated herein by reference). In addition, release of biotherapeutic compounds from this delivery system is controllable through the use of covalent crosslinking and the addition of antifibrinolytic agents to the fibrin polymer (Uchino et al., Fibrinolysis 5:93-98, 1991, incorporated herein by reference).

More simplified delivery systems for use within the invention include the use of cationic lipids as delivery vehicles or carriers, which can be effectively employed to provide an electrostatic interaction between the lipid carrier and such charged biologically active agents as proteins and polyanionic nucleic acids (see, e.g., Hope et al., Molecular Membrane Biology 15:1-14, 1998, incorporated herein by reference). This allows efficient packaging of the drugs into a form suitable for mucosal administration and/or subsequent delivery to systemic compartments. These and related systems are particularly well suited for delivery of polymeric nucleic acids, e.g., in the form of gene constructs, antisense oligonucleotides and ribozymes. These drugs are large, usually negatively charged molecules with molecular weights on the order of 106 for a gene to 103 for an oligonucleotide. The targets for these drugs are intracellular, but their physical properties prevent them from crossing cell membranes by passive diffusion as with conventional drugs. Furthermore, unprotected DNA is degraded within minutes by nucleases present in normal plasma. To avoid inactivation by endogenous nucleases, antisense oligonucleotides and ribozymes can be chemically modified to be enzyme resistant by a variety of known methods, but plasmid DNA must ordinarily be protected by encapsulation in viral or non-viral envelopes, or condensation into a tightly packed particulate form by polycations such as proteins or cationic lipid vesicles. More recently, small unilamellar vesicles (SUVs) composed of a cationic lipid and dioleoylphosphatidylethanolamine (DOPE) have been successfully employed as vehicles for polynucleic acids, such as plasmid DNA, to form particles capable of transportation of the active polynucleotide across plasma membranes into the cytoplasm of a broad spectrum of cells. This process (referred to as lipofection or cytofection) is now widely employed as a means of introducing plasmid constructs into cells to study the effects of transient gene expression. Exemplary delivery vehicles of this type for use within the invention include cationic lipids (e.g., N-(2,3-(dioleyloxy)propyl)-N,N,N-trimethyl am-monium chloride (DOTMA)), quartemary ammonium salts (e.g., N,N-dioleyl-N,N-dimethylammonium chloride (DODAC)), cationic derivatives of cholesterol (e.g., 3□(N-(N′,N-dimethylaminoethane-carbamoyl-cholesterol (DC-chol)), and lipids characterized by multivalent headgroups (e.g., dioctadecyldimethylammonium chloride (DOGS), commercially available as Transfectam®).

Additional delivery vehicles for use within the invention include long and medium chain fatty acids, as well as surfactant mixed micelles with fatty acids (see, e.g., Muranishi, Crit. Rev. Ther. Drug Carrier Syst. 7:1-33, 1990, incorporated herein by reference). Most naturally occurring lipids in the form of esters have important implications with regard to their own transport across mucosal surfaces. Free fatty acids and their monoglycerides which have polar groups attached have been demonstrated in the form of mixed micelles to act on the intestinal barrier as penetration enhancers. This discovery of barrier modifying function of free fatty acids (carboxylic acids with a chain length varying from 12 to 20 carbon atoms) and their polar derivatives has stimulated extensive research on the application of these agents as mucosal absorption enhancers.

For use within the methods of the invention, long chain fatty acids, especially fusogenic lipids (unsaturated fatty acids and monoglycerides such as oleic acid, linoleic acid, linoleic acid, monoolein, etc.) provide useful carriers to enhance delivery of proinflammatory or anti-inflammatory binding peptides, analogs and mimetics disclosed herein. Medium chain fatty acids (C6 to C12) and monoglycerides have also been shown to have enhancing activity in intestinal drug absorption and can be adapted for use within delivery formulations and methods of the invention. In addition, sodium salts of medium and long chain fatty acids are effective delivery vehicles and absorption-enhancing agents for delivery of proinflammatory or anti-inflammatory binding peptides within the invention. Thus, fatty acids can be employed in soluble forms of sodium salts or by the addition of non-toxic surfactants, e.g., polyoxyethylated hydrogenated castor oil, sodium taurocholate, etc. Mixed micelles of naturally occurring unsaturated long chain fatty acids (oleic acid or linoleic acid) and their monoglycerides with bile salts have been shown to exhibit absorption-enhancing abilities which are basically harmless to the intestinal mucosa (see, e.g., Muranishi, Pharm. Res. 2:108-118, 1985; and Crit. Rev. Ther. drug carrier Syst. 7:1-33, 1990, each incorporated herein by reference). Other fatty acid and mixed micellar preparations that are useful within the invention include, but are not limited to, Na caprylate (C8), Na caprate (C10), Na laurate (C12) or Na oleate (C18), optionally combined with bile salts, such as glycocholate and taurocholate.

Pegylation

Additional methods and compositions provided within the invention involve chemical modification of proinflammatory or anti-inflammatory binding peptides by covalent attachment of polymeric materials, for example dextrans, polyvinyl pyrrolidones, glycopeptides, polyethylene glycol and polyamino acids. The resulting conjugated peptides retain their biological activities and solubility for clinical administration. In alternate embodiments, proinflammatory or anti-inflammatory binding peptides are conjugated to polyalkylene oxide polymers, particularly polyethylene glycols (PEG) (see, e.g., U.S. Pat. No. 4,179,337, incorporated herein by reference). Numerous reports in the literature describe the potential advantages of pegylated peptides and proteins, which often exhibit increased resistance to proteolytic degradation, increased plasma half-life, increased solubility and decreased antigenicity and immunogenicity (Nucci, et al., Advanced Drug Deliver Reviews 6:133-155, 1991; Lu et al., Int. J. Peptide Protein Res. 43:127-138, 1994, each incorporated herein by reference). A number of proteins, including L-asparaginase, strepto-kinase, insulin, interleukin-2, adenosine deamidase, L-asparaginase, interferon alpha 2b, superoxide dismutase, streptokinase, tissue plasminogen activator (tPA), urokinase, uricase, hemoglobin, TGF-beta, EGF, and other growth factors, have been conjugated to PEG and evaluated for their altered biochemical properties as therapeutics (see, e.g., Ho, et al., Drug Metabolism and Disposition 14:349-352, 1986; Abuchowski et al., Prep. Biochem. 9:205-211, 1979; and Rajagopaian et al., J. Clin. Invest. 75:413-419, 1985, Nucci et al., Adv. Drug Delivery Rev. 4:133-151, 1991, each incorporated herein by reference). Although the in vitro biological activities of pegylated proteins may be decreased, this loss in activity is usually offset by the increased in vivo half-life in the bloodstream (Nucci, et al., Advanced Drug Deliver Reviews 6:133-155, 1991, incorporated herein by reference). Accordingly, these and other polymer-coupled peptides and proteins exhibit enhanced properties, such as extended half-life and reduced immunogenicity, when administered mucoally according to the methods and formulations herein.

Several procedures have been reported for the attachment of PEG to proteins and peptides and their subsequent purification (Abuchowski et al., J. Biol. Chem. 252:3582-3586, 1977; Beauchamp et al., Anal. Biochem. 131:25-33, 1983, each incorporated herein by reference). In addition, Lu et al., Int. J. Pelptide Protein Res. 43:127-138, 1994 (incorporated herein by reference) describe various technical considerations and compare PEGylation procedures for proteins versus peptides (see also, Katre et al., Proc. Natl. Acad. Sci. USA 84:1487-1491, 1987; Becker et al., Makromol. Chem. Rapid Commun. 3:217-223, 1982; Mutter et al., Makromol. Chem. Rapid Commun. 13:151-157, 1992; Merrifield, R. B., J. Am. Chem. Soc. 85:2149-2154, 1993; Lu et al., Pentide Res. 6:142-146, 1993; Lee et al., Bioconjugate Chem. 10:973-981, 1999, Nucci et al., Adv. Drug Deliv. Rev. 6:133-151, 1991; Francis et al., J. Drug Targeting 3:321-340, 1996; Zalipsky, S., Bioconjugate Chem. 6:150-165, 1995; Clark et al., J. Biol. Chem. 271:21969-21977, 1996; Pettit et al., J. Biol. Chem. 272:2312-2318, 1997; Delgado et al., Br. J. Cancer 73:175-182, 1996; Benhar et al., Bioconjugate Chem. 5:321-326, 1994; Benhar et al., J. Biol. Chem. 269:13398-13404, 1994; Wang et al., Cancer Res. 53:4588-4594, 1993; Kinstler et al., Pharm. Res. 13:996-1002, 1996, Filpula et al., Exp. Opin. Ther. Patents 9:231-245, 1999; Pelegrin et al., Hum. Gene Ther. 9:2165-2175, 1998, each incorporated herein by reference).

Following these and other teachings in the art, the conjugation of proinflammatory or anti-inflammatory binding peptides with polyethyleneglycol polymers, is readily undertaken, with the expected result of prolonging circulating life and/or reducing immunogenicity while maintaining an acceptable level of activity of the PEGylated active agent. Amine-reactive PEG polymers for use within the invention include SC-PEG with molecular masses of 2000, 5000, 10000, 12000, and 20 000; U-PEG-10000; NHS-PEG-3400-biotin; T-PEG-5000; T-PEG-12000; and TPC-PEG-5000. Chemical conjugation chemistries for these polymers have been published (see, e.g., Zalipsky, S., Bioconjugate Chem. 6:150-165, 1995; Greenwald et al., Bioconjugate Chem. 7:638-641, 1996; Martinez et al., Macromol. Chem. Phys. 198:2489-2498, 1997; Hermanson, G. T., Bioconjugate Techniques, pp. 605-618, 1996; Whitlow et al., Protein Eng. 6:989-995, 1993; Habeeb, A. F. S. A., Anal. Biochem. 14:328-336, 1966; Zalipsky et al., Poly(ethyleneglycol) Chemistry and Biological Applications, pp. 318-341, 1997; Harlow et al., Antibodies: a Laboratory Manual, pp. 553-612, Cold Spring harbor Laboratory, Plainview, N.Y., 1988; Milenic et al, Cancer Res. 51:6363-6371, 1991; Friguet et al., J. Immunol. Methods 77:305-319, 1985, each incorporated herein by reference). While phosphate buffers are commonly employed in these protocols, the choice of borate buffers may beneficially influence the PEGylation reaction rates and resulting products.

PEGylation of biologically active peptides and proteins may be achieved by modification of carboxyl sites (e.g., aspartic acid or glutamic acid groups in addition to the carboxyl terminus). The utility of PEG-hydrazide in selective modification of carbodiimide-activated protein carboxyl groups under acidic conditions has been described (Zalipsky, S., Bioconjugate Chem. 6:150-165, 1995; Zalipsky et al., Poly(ethylenemlycol) Chemistry and Biological Applications, pp. 318-341, American Chemical Society, Washington, D.C., 1997, incorporated herein by reference). Alternatively, bifunctional PEG modification of biologically active peptides and proteins can be employed. In some procedures, charged amino acid residues, including lysine, aspartic acid, and glutamic acid, have a marked tendency to be solvent accessible on protein surfaces. Conjugation to carboxylic acid groups of proteins is a less frequently explored approach for production of protein bioconjugates. However, the hydrazide/EDC chemistry described by Zalipsky and colleagues (Zalipsky, S., Bioconjugate Chem. 6:150-165, 1995; Zalipsky et al., Poly(ethyleneglycol) Chemistry and Biological Applications, pp. 318-341, American Chemical Society, Washington, D.C., 1997, each incorporated herein by reference) offers a practical method of linking PEG polymers to protein carboxylic sites. For example, this alternate conjugation chemistry has been shown to be superior to amine linkages for PEGylation of brain-derived neurotrophic factor (BDNF) while retaining biological activity (Wu et al., Proc. Natl. Acad. Sci. U.S.A. 96:254-259, 1999, incorporated herein by reference). Maeda and colleagues have also found carboxyl-targeted PEGylation to be the preferred approach for bilirubin oxidase conjugations (Maeda et al., Poly(ethylene glycol) Chemistry. Biotechnical and Biomedical Applications, J. M. Harris, Ed., pp. 153-169, Plenum Press, New York, 1992, incorporated herein by reference).

Often, PEGylation of peptides for use within the invention involves activating PEG with a functional group that will react with lysine residues on the surface of the peptide or protein. Within certain alternate aspects of the invention, biologically active peptides and proteins are modified by PEGylation of other residues such as His, Trp, Cys, Asp, Glu, etc., without substantial loss of activity. If PEG modification of a selected peptide or protein proceeds to completion, the activity of the peptide or protein is often diminished. Therefore, PEG modification procedures herein are generally limited to partial PEGylation of the peptide or protein, resulting in less than about 50%, more commonly less than about 25%, loss of activity, while providing for substantially increased half-life (e.g., serum half life) and a substantially decreased effective dose requirement of the PEGylated active agent.

Other Stabilizing Modifications of Active Agents

In addition to PEGylation, proinflammatory or anti-inflammatory binding peptides can be modified to enhance circulating half-life by shielding the proinflammatory or anti-inflammatory binding peptide via conjugation to other known protecting or stabilizing compounds, for example by the creation of fusion proteins with an active peptide, protein, analog or mimetic linked to one or more carrier proteins, such as one or more immunoglobulin chains (see, e.g., U.S. Pat. Nos. 5,750,375; 5,843,725; 5,567,584 and 6,018,026, each incorporated herein by reference). These modifications will decrease the degradation, sequestration or clearance of the peptide and result in a longer half-life in a physiological environment (e.g., in the circulatory system, or at a mucosal surface). The active agents modified by these and other stabilizing conjugations methods are therefore useful with enhanced efficacy within the methods of the invention. In particular, the peptides thus modified maintain activity for greater periods at a target site of delivery or action compared to the unmodified active agent. Even when the active agent is thus modified, it retains substantial biological activity in comparison to a biological activity of the unmodified compound.

In other aspects of the invention, proinflammatory or anti-inflammatory binding peptides are conjugated for enhanced stability with relatively low molecular weight compounds, such as aminolethicin, fatty acids, vitamin B12, and glycosides (see, e.g., Igarishi et al., Proc. Int. Svmp. Control. Rel. Bioact. Materials, 17, 366, (1990). Additional exemplary modified peptides for use within the compositions and methods of the invention will be beneficially modified for in vivo use by:

(a) chemical or recombinant DNA methods to link mammalian signal peptides (see, e.g., Lin et al., J. Biol. Chem. 270:14255, 1995, incorporated herein by reference) or bacterial peptides (see, e.g., Joliot et al., Proc. Natl. Acad. Sci. USA 88:1864, 1991, incorporated herein by reference) to the active peptide, which serves to direct the active peptide or protein across cytoplasmic and organellar membranes and/or traffic the active peptide or protein to the a desired intracellular compartment (e.g., the endoplasmic reticulum (ER) of antigen presenting cells (APCs), such as dendritic cells for enhanced CTL induction);

(b) addition of a biotin residue to the active peptide which serves to direct the active conjugate across cell membranes by virtue of its ability to bind specifically (i.e., with a binding affinity greater than about 10⁶, 10⁷, 10⁸, 10⁹, or 10¹⁰ M⁻¹) to a translocator present on the surface of cells (Chen et al., Analytical Biochem. 227:168, 1995, incorporated herein by reference);

(c) addition at either or both the amino- and carboxy-terminal ends of the active peptide of a blocking agent in order to increase stability in vivo. This can be useful in situations in which the termini of the active peptide or protein tend to be degraded by proteases prior to cellular uptake or during intracellular trafficking. Such blocking agents can include, without limitation, additional related or unrelated peptide sequences that can be attached to the amino and/or carboxy terminal residues of the therapeutic polypeptide or peptide to be administered. This can be done either chemically during the synthesis of the peptide or by recombinant DNA technology. Blocking agents such as pyroglutamic acid or other molecules known to those skilled in the art can also be attached to the amino and/or carboxy terminal residues, or the amino group at the amino terminus or carboxyl group at the carboxy terminus can be replaced with a different moiety.

Prodrug Modifications

Yet another processing and formulation strategy useful within the invention is that of prodrug modification. By transiently (i.e., bioreversibly) derivatizing such groups as carboxyl, hydroxyl, and amino groups in small organic molecules, the undesirable physicochemical characteristics (e.g., charge, hydrogen bonding potential, etc. that diminish mucosal penetration) of these molecules can be “masked” without permanently altering the pharmacological properties of the molecule. Bioreversible prodrug derivatives of therapeutic small molecule drugs have been shown to improve the physicochemical (e.g., solubility, lipophilicity) properties of numerous exemplary therapeutics, particularly those that contain hydroxyl and carboxylic acid groups.

One approach to making prodrugs of amine-containing active agents, such as the peptides of the invention, is through the acylation of the amino group. Optionally, the use of acyloxyalkoxycarbamate derivatives of amines as prodrugs has been discussed. 3-(2′-hydroxy-4′,6′-dimethylphenyl)-3,3-dimethylpropionic acid has been employed to prepare linear, esterase-, phosphatase-, and dehydrogenase-sensitive prodrugs of amines (Amsberry et al., Pharm. Res. 8:455-461, 1991; Wolfe et al., J. Org. Chem. 57:6138, 1992, each incorporated herein by reference). These systems have been shown to degrade through a two-step mechanism, with the first step being the slow, rate-determining enzyme-catalyzed (esterase, phosphatase, or dehydrogenase) step, and the second step being a rapid (t_{1/2}=100 sec., pH 7.4, 37° C.) chemical step (Amsberry et al., J. Org. Chem. 55:5867-5877, 1990, incorporated herein by reference). Interestingly, the phosphatase-sensitive system has recently been employed to prepare a very water-soluble (greater than 10 mg/ml) prodrug of TAXOL which shows significant antitumor activity in vivo. These and other prodrug modification systems and resultant therapeutic agents are useful within the methods and compositions of the invention.

For the purpose of preparing prodrugs of peptides that are useful within the invention, U.S. Pat. No. 5,672,584 (incorporated herein by reference) further describes the preparation and use of cyclic prodrugs of biologically active peptides and peptide nucleic acids (PNAs). To produce these cyclic prodrugs, the N-terminal amino group and the C-terminal carboxyl group of a biologically active peptide or PNA is linked via a linker, or the C-terminal carboxyl group of the peptide is linked to a side chain amino group or a side chain hydroxyl group via a linker, or the N-terminal amino group of said peptide is linked to a side chain carboxyl group via a linker, or a side chain carboxyl group of said peptide is linked to a side chain amino group or a side chain hydroxyl group via a linker. Useful linkers in this context include 3-(2′-hydroxy-4′,6′-dimethyl phenyl)-3,3-dimethyl propionic acid linkers and its derivatives, and acyloxyalkoxy derivatives. The incorporated disclosure provides methods useful for the production and characterization of cyclic prodrugs synthesized from linear peptides, e.g., opioid peptides that exhibit advantageous physicochemical features (e.g., reduced size, intramolecular hydrogen bond, and amphophilic characteristics) for enhanced cell membrane permeability and metabolic stability. These methods for peptide prodrug modification are also useful to prepare modified peptide therapeutic derivatives for use within the methods and compositions of the invention.

Purification and Preparation

The peptides of the invention can be prepared in a wide variety of ways. Because of their relatively short size, the peptides can be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co. (1984); Tam et al., J. Am. Chem. Soc. 105:6442 (1983); Merrifield, Science 232:341-347 (1986); and Barany and Merrifield, The Peptides, Gross and Meienhofer, eds., Academic Press, New York, pp. 1-284 (1979), each of which is incorporated herein by reference.

Alternatively, recombinant DNA technology may be employed wherein a nucleotide sequence which encodes a proinflammatory or anti-inflammatory binding peptide of interest is inserted into an expression vector, transformed or transfected into an appropriate host cell and cultivated under conditions suitable for expression. These procedures are generally known in the art, as described generally in Sambrook et al., Molecular Cloning, A Laboratory Manual, cold Spring Harbor Press, Cold Spring Harbor, N.Y. (1982), and Ausubel et al., (ed.) Current Protocols in Molecular Biology, John Wiley and Sons, Inc., New York (1987), and U.S. Pat. Nos. 4,237,224, 4,273,875, 4,431,739, 4,363,877 and 4,428,941, for example, which disclosures are incorporated herein by reference. Thus, fusion proteins which comprise one or more peptide sequences of the invention can be used to present the proinflammatory or anti-inflammatory binding peptide.

As the coding sequence for peptides of the length contemplated herein can be synthesized by chemical techniques, for example, the phosphotriester method of Matteucci et al., J. Am. Chem. Soc. 103:3185 (1981), modification can be made simply by substituting the appropriate base(s) for those encoding the native peptide sequence. The coding sequence can then be provided with appropriate linkers and ligated into expression vectors commonly available in the art, and the vectors used to transform suitable hosts to produce the desired fusion protein. A number of such vectors and suitable host systems are now available. For expression of the fusion proteins, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed.into suitable bacterial hosts. Of course, yeast or mammalian cell hosts may also be used, employing suitable vectors and control sequences.

The peptides of the present invention and pharmaceutical and vaccine compositions thereof are useful for administration to mammals, particularly humans, to treat and/or prevent a variety of diseases and conditions. The proinflammatory or anti-inflammatory binding peptides are generally provided for direct administration to subjects in a substantially purified form. The term “substantially purified” as used herein, is intended to refer to a peptide, protein, nucleic acid or other compound that is isolated in whole or in part from naturally associated proteins and other contaminants, wherein the peptide, protein, nucleic acid or other active compound is purified to a measurable degree relative to its naturally-occurring state, e.g., relative to its purity within a cell extract.

In certain embodiments, the term “substantially purified” refers to a peptide composition that has been isolated from a cell, cell culture medium, or other crude preparation and subjected to fractionation to remove various components of the initial preparation, such as proteins, cellular debris, and other components. Of course, such purified preparations may include materials in covalent association with the active agent, such as glycoside residues or materials admixed or conjugated with the active agent, which may be desired to yield a modified derivative or analog of the active agent or produce a combinatorial therapeutic formulation, conjugate, fusion protein or the like. The term purified thus includes such desired products as peptide and protein analogs or mimetics or other biologically active compounds wherein additional compounds or moieties such as polyethylene glycol, biotin or other moieties are bound to the active agent in order to allow for the attachment of other compounds and/or provide for formulations useful in therapeutic treatment or diagnostic procedures.

As applied to polynucleotides, the term substantially purified denotes that the polynucleotide is free of substances normally accompanying it, but may include additional sequence at the 5′ and/or 3′ end of the coding sequence which might result, for example, from reverse transcription of the noncoding portions of a message when the DNA is derived from a cDNA library, or might include the reverse transcript for the signal sequence as well as the mature protein encoding sequence.

When referring to peptides, proteins and peptide analogs (including peptide fusions with other peptides and/or proteins) of the invention, the term substantially purified typically means a composition which is partially to completely free of other cellular components with which the peptides, proteins or analogs are associated in a non-purified, e.g., native state or environment. Purified peptides and proteins are generally in a homogeneous or nearly homogenous state although it can be either in a dry state or in an aqueous solution. Purity and homogeneity are typically determined using analytical chemistry techniques such as polyacrylamide gel electrophoresis or high performance liquid chromatography.

Generally, substantially purified peptides, proteins and other active compounds for use within the invention comprise more than 80% of all macromolecular species present in a preparation prior to admixture or formulation of the peptide, protein or other active agent with a pharmaceutical carrier, excipient, buffer, absorption enhancing agent, stabilizer, preservative, adjuvant or other co-ingredient in a complete pharmaceutical formulation for therapeutic administration. More typically, the peptide or other active agent is purified to represent greater than 90%, often greater than 95% of all macromolecular species present in a purified preparation prior to admixture with other formulation ingredients. In other cases, the purified preparation of active agent may be essentially homogeneous, wherein other macromolecular species are not detectable by conventional techniques.

Various techniques suitable for use in peptide and protein purification are well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and/or affinity chromatography; isoelectric focusing; gel electrophoresis; and combinations of such and other techniques. Particularly useful purification methods include selective precipitation with such substances as ammonium sulfate; column chromatography; affinity methods, including immunopurification methods; and others (See, for example, R. Scopes, Protein Purification: Principles and Practice, Springer-Verlag: New York, 1982, incorporated herein by reference). In general, biologically active peptides and proteins can be extracted from tissues or cell cultures that express the peptides and then immunoprecipitated, where after the peptides and proteins can be further purified by standard protein chemistry/chromatographic methods.

Peptides and proteins used in the methods and compositions of the invention can be obtained by a variety of means. Many peptides and proteins can be readily obtained in purified form from commercial sources. Smaller peptides (less than 100 amino acids long) can be conveniently synthesized by standard chemical methods familiar to those skilled in the art (e.g., see Creighton, Proteins: Structures and Molecular Principles, W.H. Freeman and Co., N.Y., 1983). Larger peptides (longer than 100 amino acids) can be produced by a number of methods including recombinant DNA technology (See, for example, the techniques described in Sambrook et al., Molecular Cloning, A Laboratory Manual, Cold Spring Harbor Press, N.Y., 1989; and Ausubel et al., eds., Current Protocols in Molecular Biology, Green Publishing Associates, Inc., and John Wiley & Sons, Inc., N.Y, 1989, each incorporated herein by reference). Alternatively, RNA encoding the proteins can be chemically synthesized. See, for example, the techniques described in Oligonucleotide Synthesis, Gait, M. J., ed., IRL Press, Oxford, 1984 (incorporated herein by reference).

In certain embodiments of the invention, biologically active peptides or proteins will be constructed using peptide synthetic techniques, such as solid phase peptide synthesis (Merrifield synthesis) and the like, or by recombinant DNA techniques, that are well known in the art. Peptide and protein analogs and mimetics may also be produced according to such methods. Techniques for making substitution mutations at predetermined sites in DNA include for example M13 mutagenesis. Manipulation of DNA sequences to produce substitutional, insertional, or deletional variants are conveniently described elsewhere, such as in Sambrook et al. (Molecular Cloning: A Laboratory Manual, Cold Spring Harbor Laboratories, Cold Spring Harbor, N.Y., 1989). In accordance with these and related teachings, defined mutations can be introduced into a biologically active peptide or protein to generate analogs and mimetics of interest by a variety of conventional techniques, e.g., site-directed mutagenesis of a cDNA copy of a portion of a gene encoding a selected peptide fragment, domain or motif. This can be achieved through and intermediate of single-stranded form, such as using the MUTA-gen® kit of Bio-Rad Laboratories (Richmond, Calif.), or a method using the double-stranded plasmid directly as a template such as the Chameleon® mutagenesis kit of Strategene (La Jolla, Calif.), or by the polymerase chain reaction employing either an oligonucleotide primer or a template which contains the mutation(s) of interest. A mutated subfragment can then be assembled into a complete peptide analog-encoding cDNA. A variety of other mutagenesis techniques are known and can be routinely adapted for use in producing mutations in biologically active peptides and proteins of interest for use within the invention.

Formulation and Adminstration

Proinflammatory or anti-inflammatory binding peptides are typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

For prophylactic and treatment purposes, the proinflammatory or anti-inflammatory binding peptides disclosed herein may be administered to the subject via any suitable route of administration, including intravenous, subcutaneous, intratumoral, intrapulmonary, perfusion, etc. For therapeutic purposes, the peptides of the invention can also be expressed by attenuated viral vectors or other gene therapy delivery constructs. Such vectors as vaccinia or fowlpox are exemplary of these widely known tools. This approach involves the use of, e.g., vaccinia virus as a vector to express nucleotide sequences that encode the peptide(s) (or conjugates) of the invention. Upon introduction to a target site (e.g., intratumoral site, site of inflammation, or virally infected site, the recombinant vaccinia virus expresses the proinflammatory or anti-inflammatory binding peptide, and thereby modulates a proinflammatory or anti-inflammatory immune response. Vaccinia vectors and methods useful in immunization protocols are described in, e.g., U.S. Pat. No. 4,722,848 (incorporated herein by reference). Another useful vector is BCG (bacille Calmette Guerin). BCG vectors are described in Stover et al. (Nature 351:456-460, 1991 (incorporated herein by reference). A wide variety of other vectors useful for therapeutic administration or immunization of the peptides of the invention, e.g., Salmonella typhi vectors and the like, will be apparent to those skilled in the art from the description herein.

The proinflammatory or anti-inflammatory binding peptides may be administered in a single bolus delivery, via continuous delivery (e.g., continuous transdermal, mucosal, or intravenous delivery) over an extended time period, or in a repeated administration protocol (e.g., by an hourly, daily or weekly, repeated administration protocol). In this context, a therapeutically effective dosage of the proinflammatory or anti-inflammatory binding peptide(s) may include repeated doses within a prolonged prophylaxis or treatment regimen, that will yield clinically significant results to alleviate one or more symptoms or detectable conditions associated with a targeted disease or condition as set forth above. Determination of effective dosages in this context is typically based on animal model studies followed up by human clinical trials and is guided by determining effective dosages and administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the proinflammatory or anti-inflammatory binding peptide(s). In alternative embodiments, an “effective amount” or “effective dose” of the biologically active agent(s) may simply inhibit or enhance one or more selected biological activity(ies) correlated with a disease or condition, as set forth above, for either therapeutic or diagnostic purposes.

The actual dosage of proinflammatory or anti-inflammatory binding peptides will of course vary according to factors such as the disease indication and particular status of the subject (e.g., the subject's age, size, fitness, extent of symptoms, susceptibility factors, etc), time and route of administration, other drugs or treatments being administered concurrently, as well as the specific pharmacology of the biologically active agent(s) for eliciting the desired activity or biological response in the subject. Dosage regimens may be adjusted to provide an optimum prophylactic or therapeutic response. A therapeutically effective amount is also one in which any toxic or detrimental side effects of the proinflammatory or anti-inflammatory binding peptide is outweighed in clinical terms by therapeutically beneficial effects. A non-limiting range for a therapeutically effective amount of a biologically active agent within the methods and formulations of the invention is 0.01 μg/kg-10 mg/kg, more typically between about 0.05 and 5 mg/kg, and in certain embodiments between about 0.2 and 2 mg/kg. Dosages within this range can be achieved by single or multiple administrations, including, e.g., multiple administrations per day, daily or weekly administrations. Per administration, it is desirable to administer at least one microgram of the proinflammatory or anti-inflammatory binding peptide, more typically between about 10 μg and 5.0 mg, and in certain embodiments between about 100 μg and 1.0 or 2.0 mg to an average human subject. It is to be further noted that for each particular subject, specific dosage regimens should be evaluated and adjusted over time according to the individual need and professional judgment of the person administering or supervising the administration of the permeabilizing peptide(s) and other biologically active agent(s).

Dosage of proinflammatory or anti-inflammatory binding peptides may be varied by the attending clinician to maintain a desired concentration at the target site. For example, a selected local concentration of the biologically active agent in the bloodstream or CNS may be about 1-50 nanomoles per liter, sometimes between about 1.0 nanomole per liter and 10, 15 or 25 nanomoles per liter, depending on the subject's status and projected or measured response. Higher or lower concentrations may be selected based on the mode of delivery, e.g., mucosal versus intravenous or subcutaneous delivery. Dosage should also be adjusted based on the release rate of the administered formulation, e.g., of a nasal spray versus powder, sustained release oral versus injected particulate or transdermal delivery formulations, etc. To achieve the same serum concentration level, for example, slow-release particles with a release rate of 5 nanomolar (under standard conditions) would be administered at about twice the dosage of particles with a release rate of 10 nanomolar.

Additional guidance as to particular dosages for selected biologically active agents for use within the invention may be found widely disseminated in the literature.

The invention is further illustrated by the following specific examples that are not intended in any way to limit the scope of the invention.

The following Examples document the discovery that HLA-E molecules can also can bind peptides derived from an exemplary stress-induced protein, human heat-shock protein 60 (hsp60). In accordance with the foregoing disclosure, a candidate hsp60 peptide for binding HLA-E was identified in the signal sequence (corresponding to the mitochondrial targeting sequence) of the hsp60 protein. During cellular stress, this peptide gains access to nascent HLA-E molecules and causes upregulation of HLA-E on the cell surface. Remarkably, HLA-E molecules binding these hsp60 peptides are no longer recognized by the inhibitory CD94/NKG2A receptor pair.

Thus, during normal cell growth HLA-E binds signal peptides derived from MHC class I signal sequences and such cells are protected from NK cell-mediated attack. During cellular stress, HLA-E molecules may bind predominantly peptides derived from other endogenous proteins, such as e.g. hsp60 signal peptides.

When HLA-E was transfected into K562 cells (an erythroleukemia cell line K562 is deficient in HLA class I cell surface expression) together with full-length hsp6o leader sequence, a slight increase in cell surface HLA-E expression was observed. In contrast, massive upregulation was observed if these cells were subjected to cell culture stress, e.g., in the form of high-density cell growth conditions. The fact that only a marginal upregulation of HLA-E was observed in K562 cells transfected with the same HLA-E construct (and also with a point mutated variant of the hsp60 leader sequence) clearly suggest that a critical peptide sequence found within the hsp60 leader is capable of gaining access to HLA-E. This observation points to a novel peptide presenting-role for HLA-E during cellular distress.

A CD94/NKG2A uncoupling on NK cells by HLA-E mediated presentation of stress induced peptides, points to a novel mechanism whereby NK cells (and CD94/NKG2A expressing T cells) can detect and eliminate stressed autologous cells during, for example, a sustained chronic inflammation.

The findings presented herein demonstrate that HLA-E presenting an exemplary HLA-E binding peptide from a stress-induced protein is no longer capable of engaging CD94/NKG2A inhibitory receptors—both as measured by NK cellular cytotoxicity against peptide-loaded HLA-E transfected cells, and by using tetrameric HLA-E/beta2 microglobulin/hsp60 peptide-complexes for binding to CD94/NKG2A either expressed endogenously and functionally on NK cells or expressed by cellular transfectants. These results suggest that the exemplary hsp60 peptide and other related peptides presented by HLA-E form a complex that uncouples CD94/NKG2A inhibitory receptors, which may result in cellular activation of cells bearing CD94/NKG2A receptors.

Cell Culture

K562 (human HLA-class I negative erythroleukemia), and 721.221 (human HLA-class I low B-lymphoblastoid cell) were maintained in RPMI 1640 (Life Technologies, Gaithersburg, Md.) supplemented with 10% heat-inactivated FCS, 2 mM L-glutamine, 100 U/ml penicillin, and 100 μg/ml streptomycin. Two human CD94/NKG2A⁺ (but KIR−) cytotoxic NK cell lines (NKL, kindly provided by Dr. M. Robertson, Indiana University School of Medicine, Indianapolis, Ind.), and Nishi (provided by Dr. H. Wakiguchi, Dept of Pediatrics, Kochi Medical School, Japan) were grown in IMDM supplemented with 7% pooled heat-inactivated human AB+ serum, 200 U IL-2/ml (PeproTech Inc, Rocky Hill N.J.), 2 mM L-glutamine, 100 U/ml penicillin, and 100 μg/ml streptomycin (Life Technologies). Ba/F3 cells co-transfected with CD94 and NKG2A, CD94, DAP-12 and NKG2C-GFP or CD94 and DAP-12 have been described previously (Lanier et al., Immunity 6:371-378, 1997, incorporated herein by reference). HB-120 (pan-HLA class I specific hybridoma) was obtained from American Type Culture Collection, Rockville, Md., and was cultured in DMEM supplemented with 10% FCS, 2 mM L-glutamine, sodium pyruvate, HAT, 100 U/ml penicillin, 100 μg/ml streptomycin (Life Technologies).

Peptides, HLA-E Stabilization, and Cell Culture Stress Assays

Synthetic peptides, purchased from Research Genetics, were dissolved in PBS. The peptides used were B7sp (VMAPRTVLL) (SEC ID NO: 3), hsp60sp (QMRPVSRVL) (SEC ID NO: 2), B7 R5V (VMAPVTVLL) (SEC ID NO: 14), hsp60 V5R (QMRPRSRVL) (SEC ID NO: 3), and P18I10 (RGPGRAFVTI) (SEC ID NO: 113) (all from Research Genetics, Huntsville, Ala.). Cells and their HLA-E transfected derivatives were incubated with synthetic peptides (3-300 μM) at 26° C. for 15-20 hours in serum-free AIM-V medium (GibcoBRL, Paisley Scotland) at a concentration of 1-3.10⁶ cells/ml. Cells were then harvested, washed in PBS, stained with mAbs and analyzed by flow cytometry. Cells were subjected to stress by allowing them to grow at increasing cell density.

Briefly, cell cultures were set up at the cell concentration of 0.2.10⁶ cells/ml at different time points for a period of up to 6 days. At the end point, cell concentration and viability were determined by trypan blue exclusion. The expression of cell-surface HLA class I molecules was assessed by flow cytometry. Cell cultures with viability higher than 90% and at three different densities were selected as targets for cytotoxic assays. HLA-E Tetramer Production

HLA-E tetrameric complexes were generated as previously described (Michaëlsson et al., Eur. J. Immunol. 30:300, 2000; Braud et al., Nature 391:795-799, 1991, each incorporated herein by reference). Briefly, HLA-E and □₂-microglobulin (□₂m) were overexpressed in E. coli BL21 pLysS, purified from inclusion bodies, solubilized into a 8M urea solution, and then refolded by dilution in vitro with synthetic peptides (B7sp, hsp60sp, B7 R5V or hsp60 V5R) (Research Genetics). Complexes of the HLA-E heavy chain, □₂m and peptide were purified by size exclusion chromatography on a Superose12 column (Amersham-Pharmacia Biotech), biotinylated with BirA enzyme (Avidity, Denver Colo.) according to the instructions of the manufacturer, then quickly frozen and stored at −80° C. Tetrameric HLA-E complexes were generated by mixing biotinylated monomers with streptavidin-phycoerythrin (Molecular Probes, Leiden, Netherlands) at a 4:1 molar ratio. Similar quality of the different tetramers was verified by gel-shift assays, as well as by staining a pan-HLA specific hybridoma (HB-120).

Antibodies and Flow Cytometry

Monoclonal antibodies (Mabs) used were: DX22 (anti-CD94, DNAX, Palo Alto, Calif.), anti-NKG2A (Z199, provided by Dr. Lorenzo Moretta, Istituto Nazionale per la Ricerca sul Cancro, Genova, Italy), CD56 (B159, BD Pharmingen), anti-MHC class I mabs (DX7, DNAX) and W6/32 (American Type Culture Collection). The 3H5 (ariti-MICA) and 3D12 (anti-HLA-E) mAbs were provided by Drs. T. Spies and D. Geraghty, respectively (Fred Hutchinson Cancer Center, Seattle, USA). Anti-hsp60 (ML30) was provided by from Dr. J. Ivanyi (University of London, England). Anti-MICB (7C5) was generated in our laboratory by immnunizing mice with P815 cells stable transfected with a pCDNA3 expression vector containing an N-terminal CD8 leader peptide followed by a FLAG epitope and the extracellular, transmembrane and cytoplasmic MICB cDNA. Hybridoma 7C5 (anti-MICB) was selected and shown to bind 721.221 and P815 cells transfected with MICB*002 cDNA expression vectors, whereas untransfected or control transfected cells as well as MICA*005 transfected cells were negative. Second-step reagents were FITC- and PE-conjugated goat anti-mouse IgG (both from Dakopatts, Glostrup, Denmark). DAK-GO1 was used as negative control mAbs for triple-colour (Dakopatts). Cells were analyzed on a FACScan# (Becton Dickinson, San Jose, Calif.). Immunofluorescence staining was conducted according to standard protocols. Briefly, K562 cells transfected with wild type (wt) or mutant full-length hsp60 signal peptide-GFP were stained with the nuclear stain Hoechst33342 for 30 min at 37° C. and the mitochondrial dye tetramethylrhodamine ethyl ester (TMRE) for 15 min at 37° C., followed by 3 washing steps. Cells were analyzed using a Nikon Eclipse E400 universal microscope connected to a Hamamatsu C4742-98 digital camera. Appropriate filters for immunofluorescence analysis of labeled cells were used and images were acquired using Jasc Paint Shop Pro 6.0 and imported into Adobe Photoshop#.

Expression Vectors and Generation of Transfected Cells

Synthesized sense and anti-sense dna coding for the full-length hsp60 signal peptide flanked by a 5′Eco RI/3′ BamHI site (5′CGGAATTCATGCTTCGGTTACCCACAGT CTTTCGCCAGATGAGACCGGTGTCCAGGGTACTGGCTCCTCATCTCACTCGGGCTTAT GGATCCGC3′) were purchased from Interactiva (Ulm, Germany). The annealed and digested product was ligated into pEGFP-N3 expression vector (Clontech, Palo Alto, USA). The triplet coding for a Met-Residue at position 11 in the hsp60 signal-peptide was mutated to a triplet coding for a Gly-Residue using the following oligonucleotide primer: 5′CAGTCTTTCGCCAGGGGAGACCGGTGTCCAG-3′ using a site-directed mutagenesis kit according to the manufacturers recommendations (QUIKCHANGE™, Stratagene, La Jolla, Calif.) and verified by sequencing. HLA-E*0101 and HLA-E*01033 cDNA encoding plasmids (pcDNA3) were provided by Drs. M. Ullbrecht and E. Weiss (Institut Fuer Anthropologie Und Humangenetik, Munich, Germany). 721.221 and K562 cells were transfected by electroporation (Gene Pulser, Biorad, Hercules Calif.) according to standard protocols. For transient co-transfection experiments with HLA-E and the chimeric GFP encoding plasmids we used a ratio of 10:1 (HLA-E:GFP). Transfected cells were selected in complete medium supplemented with 1 mg/ml G418 (BioRad). Stable transfected cells were isolated by flow cytometry (FACSCAN#) on the basis of their green fluorescent properties.

NK Cell-Mediated Cytotoxicity Assays

NK cell-mediated cytotoxicity was measured using a 2 hours standard ⁵¹Cr radioisotope release assay. Briefly, target cells were incubated for 15-20 hours at 26° C. with the various peptides at concentrations ranging from 1-300 μM, and then labeled with ⁵¹Cr. Peptides were washed away prior to setting up the assays, except in some experiments where the non-protective hsp60sp, B7 R5V and hsp60 V5R was kept throughout the assay to assure higher levels of HLA-E expression, as compared to targets incubated with the protective B7sp. In mAb blocking experiments, cells were preincubated with mouse serum, or an irrelevant isotype matched antibody to block Fc-receptors. Blocking of either target or effector cells with mAbs was performed at 4° C., and the antibodies were also included during the assays.

### EXAMPLE I

Hsp60sp Stabilizes HLA-E Sell Surface Expression

To identify peptides derived from human hsp60 with a potential to bind HLA-E, the full length amino acid sequence of hsp60 was scanned for peptides displaying an HLA-E permissive motif (methionine at position 2 followed by either a leucine or isoleucine at position 9 at the C-terminus). Among four such peptides identified (FIG. 1; Table 3), one (QMRPVSRVL (SEC ID NO: 2), designated hsp60sp) was initially selected based on its location within the hsp60 leader sequence. In addition, hsp60sp not only bears a methionine at position 2 and a leucine at position 9, but also shares amino acids at position 4 and 8 in common with some peptides known to efficiently bind to HLA-E (Table 1). In particular, four out of the nine amino acids in hsp60sp are shared with some peptides found in HLA class I leader sequences (e.g., HLA-A*0201, and -A*3401, Table 1).

peptide and HLA-E interactions have demonstrated that the presence of an HLA-E binding peptide, either provided in a transfected cDNA expression plasmid, or by exogenous addition of synthetic peptides, is sufficient to stabilize and upregulate HLA-E cell surface expression to levels detectable by flow cytometry (Braud et al., Nature 391:795-799, 1991; Lee et al., Proc. Natl. Acad. Sci. USA 95:5199, 1998; Borrego et al., J. Exp. Med. 187:813, 1998, each incorporated herein by reference). To test whether the hsp60-derived peptides were able to bind HLA-E, stabilized HLA-E cell surface expression was stabilized with the different synthetic peptides, by overnight incubation at 26° C. For this purpose MHC class I-deficient cell lines such as 721.221 (which lack HLA-A, -B, -C, and -G, but express HLA-E and -F intracellularly), as well as K562 cells transfected with HLA-E*01033 (K562-E*01033) or HLA-E*0101 were employed. The 721.221 cells and HLA-E transfected, but not untransfected, K562 cells express low levels of HLA-E at the cell surface during normal cell growth. These base levels of HLA-E expression suggest the presence of minute amounts of intracellular peptides, enough to stabilize nascent HLA-E molecules.

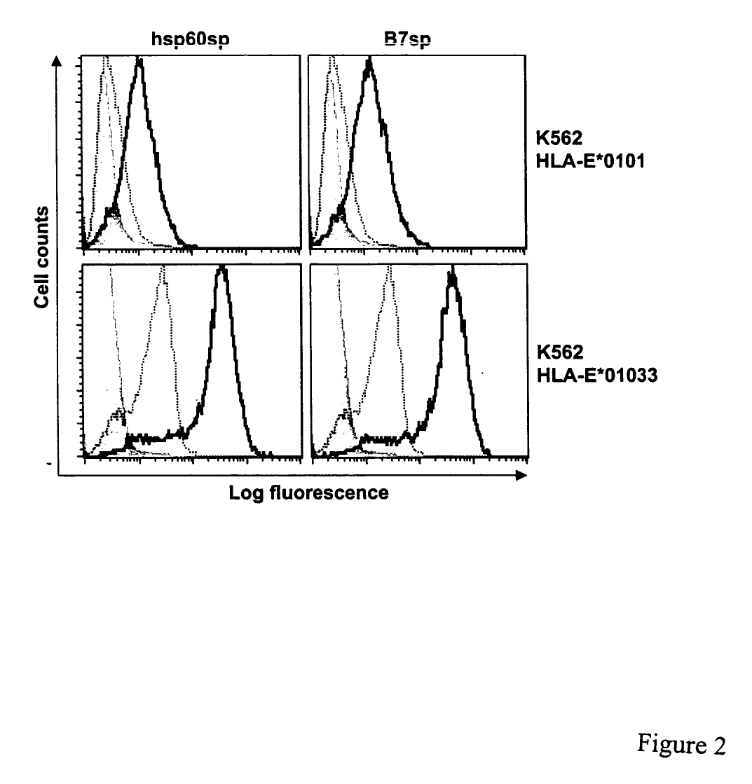

As shown in FIG. 2, using hsp60sp, a substantial increase in HLA-E expression was observed in both HLA-E*01033 and HLA-E*0101 transfected K562 cells. The levels of HLA-E expression after loading with hsp60sp were comparable to the levels of cells loaded with a peptide derived from the leader sequence of HLA-B*0701 (B7sp, VMAPRTVLL) (SEC ID NO: 3) (FIG. 2). However, at 37° C. the HLA-E/hsp60sp complexes dissociated faster than the HLA-E/B7sp, reaching base levels after approximately. In addition to hsp60sp, the hsp60.4 peptide (GMKFDRGYI) (SEC ID NO: 11) was also capable of stabilizing HLA-E molecules on transfected K562 cells as well as on 721.221 cells. This peptide has previously been shown to also bind to mouse Qa-1^{b} molecules (Lo et al., Nature Med. 6:215, 2000, incorporated herein by reference). HLA-E stabilization was not observed with the other two hsp60 derived peptides (hsp60.2 and hsp60.3; Table I), possibly due to poor solubility in the assay medium.

### EXAMPLE II

Hsp60 Signal Peptide Gains Access to HLA-E Intracellularly and HLA-E/hsp60sp Levels are Up-Regulated During Cellular Stress

Hsp60 is a mitochondrial matrix protein, which is encoded within the genomic DNA (Bukau et al., Cell 923:351, 1998; Itoh et al., J. Biol. Chem. 270:13429, 1995, each incorporated herein by reference). It is synthesized as a precursor protein with an N-terminal mitochondrial targeting sequence consisting of 26 amino acids (hsp60L, see FIG. 1). Biochemical studies have established that cleavage of the hsp60L requires import of the precursor protein into the mitochondrial matrix, and that this cleavage is unlikely to occur in the cytosol, since no mitochondrial import of hsp60 is observed in the absence of the hsp60L (Singh et al., Biochem. Biophys. Res. Commun. 1692:391, 1990, incorporated herein by reference). The final destination for the hsp60L after its cleavage is unknown. Upon stress, hsp60 is regulated by increased transcription as well as by post-transcriptional events affecting its intracellular levels and distribution (Belles et al., Infect. Immun. 67:4191, 1999; Samali et al., Embo J. 18:2040, 1999; Feng et al., Blood 97:3505, 2001; Soltys et al., Exp. Cell. Res. 222:16, 1996, each incorporated herein by reference).

To follow the localization of the hsp60L, and particularly to determine whether the hsp60sp can gain access to HLA-E molecules, a model system based on K562 cells transfected with chimeric constructs containing either the wild-type hsp60L, or a mutated variant in which the methionine at position 11 was substituted by a glycine was developed. The noted methionine residue corresponds to position 2 in hsp60sp nonamer, and is required for stable binding to HLA-E. The wild-type and mutated hsp60L were grafted in frame onto the N-terminus of green fluorescent protein (GFP) to ensure that, upon transfection, green fluorescent cells also translate each of the individual hsp60 leader sequences. Following transfection, GFP was localized inside the mitochondria in the two hsp60L-GFP transfected cell lines, indicating that the substitution of methionine at position 11 did not alter the transport into the mitochondria. GFP showed no obvious subcellular localization when the GFP gene was transfected alone.

Previously, it has been reported that cell surface levels of mouse Qa-1^{b} molecules are substantially upregulated during cellular stress (Imani et al., Proc. Natl. Acad. Sci. USA 88:10475, 1991, incorporated herein by reference). Considering the homology between Qa-1^{b} and HLA-E, both in terms of sequence, biological function and peptide binding specificity, experiments were designed to test whether the nonameric peptide located inside the mitochondrial targeting sequence of hsp60 may ultimately gain access to HLA-E, particularly under conditions of cellular stress. To this end K562 cells were co-transfected with an HLA-E*01033-encoding plasmid together with either the wild-type hsp60L-GFP construct or with its mutated variant. HLA-E cell surface expression of these transfectants was then monitored as the cultures were subjected to stress by means of growth at increasing cellular density.

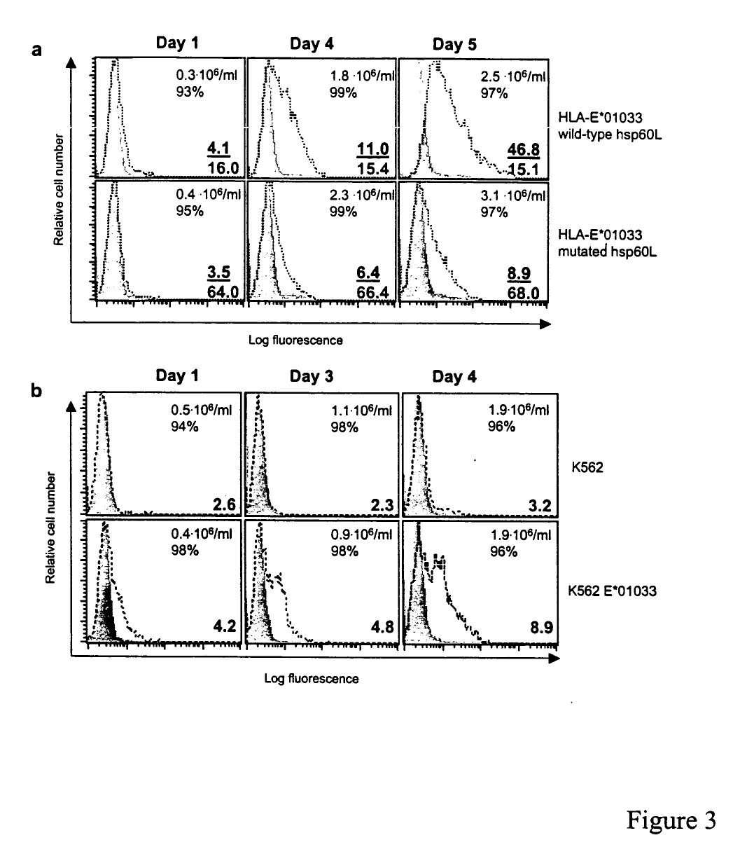

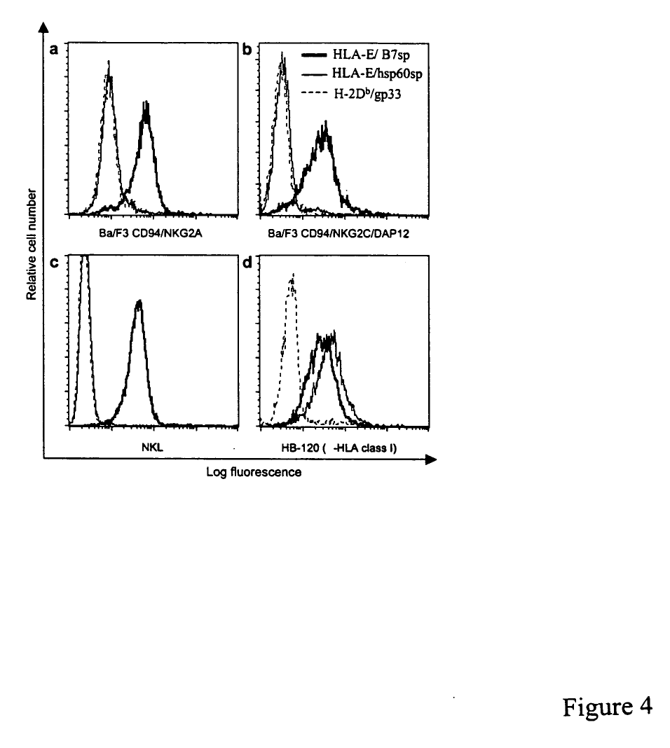

Cells transfected with the wild-type hsp60L-GFP construct consistently expressed higher levels of HLA-E than cells co-transfected with the mutant construct (FIG. 3a). It should be noted that this difference depended on the growth conditions; at day 1, the difference in HLA-E cell surface levels between cells expressing wild-type and mutated hsp60sp was moderate, while it was substantial at day 5. There was also a certain increase of HLA-E levels in the cells transfected with the mutated hsp60L-GFP construct when grown under stress versus normal conditions (FIG. 3a, day 1 versus day 5). This could be due either to a residual capacity of the mutated peptide to bind HLA-E, or by an access of endogenously derived hsp60 peptides to HLA-E. Consistent with the latter possibility, HLA-E levels increased as a consequence of culture-induced stress also in K562 cells that had been transfected with the HLA-E gene alone (FIG. 3b, lower panel), whereas untransfected K562 cells remained HLA-E negative (FIG. 3b upper panel). There remains a possibility of an influence by other HLA-E binding peptides, as well as post-transcriptional, but peptide independent, regulation of HLA-E in stressed cells. It is also noted that the K562-E*01033 cell line and the co-transfected cell lines presented in FIG. 4a were generated and selected independently, which may account for the higher HLA-E background level observed at day 1. Therefore the absolute levels of HLA-E are not necessarily directly comparable between FIGS. 3a and 3b.

The foregoing transfectant studies indicate that the stress response results in an increased accessibility of mitochondrial hsp60sp to HLA-E intracellularly, eventually causing up-regulated HLA-E/hsp60sp cell surface levels. This appears to be due, at least in part to post-transcriptional control of hsp60sp during the stress response, since both the hsp60L-GFP and HLA-E constructs used were under control of the same CMV promoter, and the GFP expression level did not change with increased cell density (FIG. 3a). Finally, although K562 constitutively express the activating NKG2D ligands MIC-A and MIC-B, further cell surface up-regulation of these stress-inducible MHC class I-like molecules during the course of these analyses was not observed. However, upregulation of other activating, stress-induced ligands, e.g. UL16 binding proteins (ULBP) cannot be excluded.

### EXAMPLE III

HLA-E Mediated Presentation of hsp60sp Abrogates Recognition by CD94/NKG2A and CD94/NKG2C Receptors

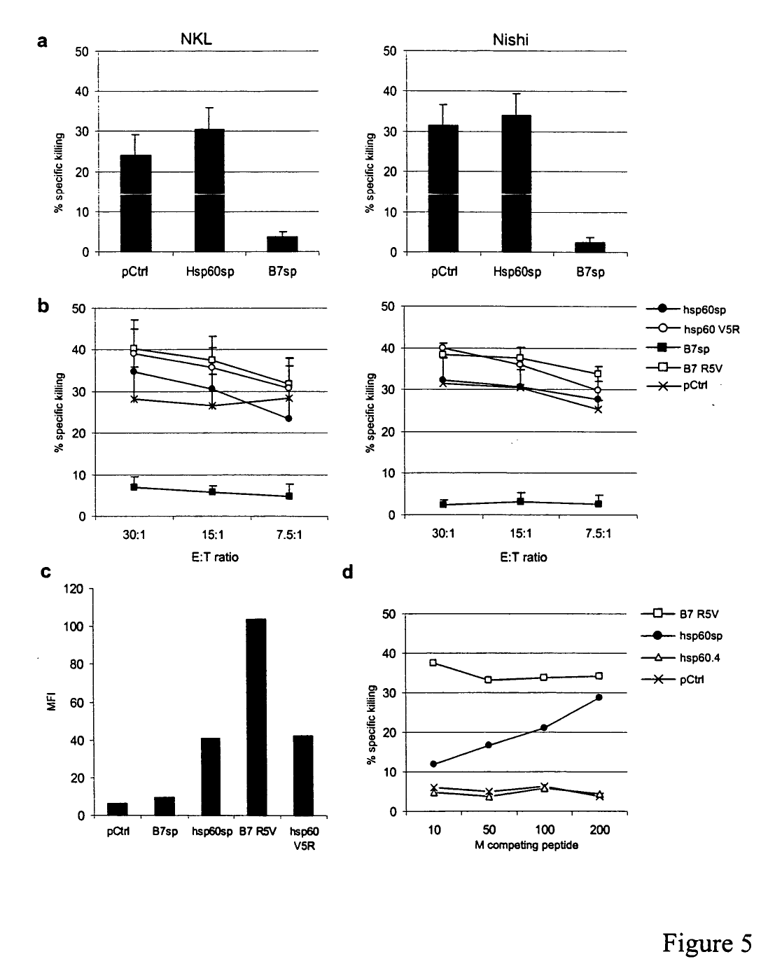

The inhibitory lectin-like receptor heterodimer CD94/NKG2A is present on approximately 50% of all NK cells in the peripheral blood both in humans and mice. This HLA-E specific receptor mediates a negative signal upon binding to HLA-E presenting various protective HLA-class I signal peptides, which results in the inactivation of NK cell effector functions. In a similar fashion, Qa-1^{b} in complex with a permissive MHC class I leader peptide is efficiently recognized by murine CD94/NKG2A receptors, suggesting evolutionary conservation in human and mice at both receptor and ligand levels. To characterize possible NK cell receptors that interact with HLA-E in complex with hsp60sp or MHC class I signal peptides, studies were designed to determine whether MHC tetrameric complexes could bind CD94INKG2 receptors expressed on transfectants and NK cells. Recombinant soluble HLA-E molecules were refolded in vitro in the presence of human β₂-microglobulin and B7sp (VMAPRTVLL) (SEC ID NO: 3) or hsp60sp (QMRPVSRVL) (SEC ID NO: 2). The refolded MHC complexes were used to create tetrameric HLA-E molecules, which enable analysis of HLA-E binding receptors. Both peptides permitted an effective refolding of HLA-E in vitro and were effectively biotinylated as analyzed by gel-shift assays. These studies demonstrated that HLA-E/B7sp tetramers efficiently bound to mouse Ba/F3 pro-B cells co-transfected with CD94 and NKG2A or CD94, NKG2C and DAP12 (FIG. 4, panels a and b). This result was confirmed by staining NK-cell lines that express the inhibitory receptor CD94/NKG2A (FIG. 4, panel c), or freshly isolated NK cells expressing predominantly the CD94/NKG2A receptor. In contrast, the HLA-E/hsp60sp tetramers failed to bind Ba/F3 pro-B cells co-transfected with either CD94/NKG2A or CD94/NKG2C/DAP12, and all NK cells examined (FIG. 5, panels a-c). However, both HLA-E/B7sp and HLA-E/hsp60sp bound to a similar extent to a control B cell hybridoma, specific for HLA class I molecules (FIG. 5, panel d). Thus, although hsp60sp can efficiently gain access to HLA-E physiologically, this complex is no longer recognized by the CD94/NKG2A and CD94/NKG2C receptors, demonstrating that they are peptide selective.

### EXAMPLE IV

HLA-E/hsp60sp Fails to Inhibit CD94/NKG2A⁺ NK Cells in Cytotoxic Assays; Critical Role for Position 5 in the Peptide

To address the functional significance of increased HLA-E/hsp60sp cell surface levels, studies were designed to determine whether cells expressing these MHC complexes were protected from killing by CD94/NKG2A⁺ NK cells. K562-E*01033 cells, incubated overnight at 26° C. with either hsp60sp or B7sp peptides, were tested as targets in 2 hours chromium release assays with the CD94/NKG2A⁺ NK cell lines Nishi and NKL as effectors. A clear protection from killing was observed when incubating the otherwise susceptible K562-E*01033 cells with B7sp, whereas incubation with hsp60sp did not result in any significant protection (FIG. 5, panel a). As noted above, hsp60sp and B7sp have different dissociation rates from HLA-E, which could account for the difference in target susceptibility. Therefore the HLA-E surface expression was monitored before and after the cytotoxic assays, to assure comparable levels of HLA-E on the targets throughout the assays.

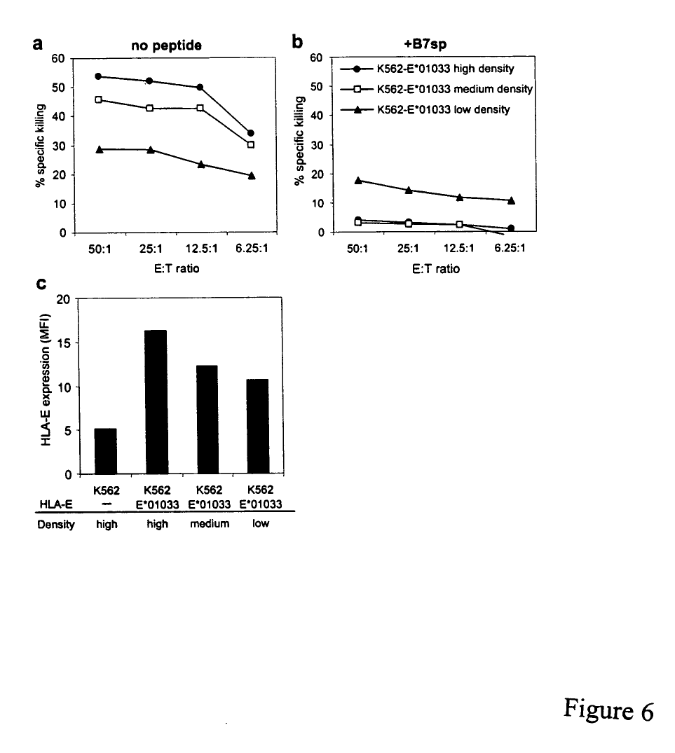

To pinpoint the residues responsible for the loss of HLA-E recognition by CD94/NKG2A, targeted mutations were introduced in the B7sp and hsp60sp. It has previously been demonstrated that a change at p5R in the Qa-1^{b} binding peptide Qdm abrogates recognition by CD94/NKG2A in the mouse (Kraft et al., J. Exp. Med. 192:613, 2000, incorporated herein by reference). To test the degree of functional conservation of the position 5 in both peptides, experimental peptides B7 R5V (VMAPVTVLL) (SEC ID NO: 114) and hsp60 V5R (QMRPRSRVL) (SEC ID NO: 2) were generated. The ability of these peptides to protect K562-E*01033 cells was tested in cytotoxic assays, as described above. K562-E*01033 cells incubated 26° C. with B7 R5V expressed high levels of HLA-E (FIG. 6, panel c), yet they were efficiently killed by CD94/NKG2A⁺ NK cells (FIG. 5, panel b). This mutation is therefore sufficient to abrogate the protective capacity of B7sp. However, the V5R mutation introduced in hsp60sp was not sufficient to restore protection from killing using the same effector cells (FIG. 5, panel b).

It has been recently recently reported that the hsp60.4 peptide (GMKFDRGYI) (SEC ID NO: 11) could bind to Qa-1^{b}, but did not induce protection from CD94/NKG2A⁺ NK cells (Gays et al. J. Immunol. 166:1601-1610, 2001, incorporated herein by reference). However, this peptide failed to compete with the protective Qdm-peptide for binding to Qa-1^{b}, even when mixed in 100,000-fold excess with 1 mM Qdm (Id.) In contrast, the present disclosure demonstrates that hsp60sp could interfere with HLA-E mediated protection by competing with MHC class I signal peptides.

Briefly, K562-E*01033 cells were incubated with 0.1 μM B7sp together with increasing concentrations of competing peptides and tested in cytotoxic assays. Cells incubated with 0.1 μM B7sp and a control peptide remained protected from killing at all concentrations tested, whereas cells incubated with 0.1 μM B7sp and hsp60sp became more susceptible to killing with increasing concentrations of hsp60sp (FIG. 6d). The B7 R5V peptide was an even stronger competitor than hsp60sp (FIG. 6d). In line with the results on Qa-1^{b} peptide binding competition as reported by Gays et al. (36), hsp60.4 was not able to compete with B7sp for binding to HLA-E (FIG. 5d).

Yet additional studies were conducted to determine whether the stress-induced HLA-E cell surface up-regulation observed in K562-E*01033 cells resulted in protection from NK cell mediated lysis. K562-E*01033 cells grown at different densities were tested as targets in a 2 hours chromium release assay with NKL and Nishi as effector cells. Despite showing increased HLA-E levels, the killing increased rather than decreased, indicating that the HLA-E molecules induced on these cells were not protective. All target cells had a viability higher than 90%, as measured by Annexin V staining and trypan blue (data not included). Moreover, and importantly, the cells grown at high density could be rescued from killing by addition of B7sp peptide (FIG. 6, panel b). This demonstrates that the increased killing was not terminally decided by the cell culture conditions, and that the HLA-E levels were sufficient for protection provided that an appropriate peptide was present. The data also subbest that HLA-E expression induced by stress is not sufficient to protect from NK cell mediated killing. It should be noted that, although K562 constitutively express MIC-A and MIC-B ligands for the activating receptor NKG2D, these are not further up-regulated by the cellular stress imposed in these assays. Therefore it is unlikely that the increased killing after cellular stress observed in some of the experiments herein is due to an increased expression of MIC-A or MIC-B. Up-regulation of other activating ligands, e.g., ULBP's, may also be responsible for the increase in killing.

Summarizing the foregoing Examples, HLA-E has been shown to binding a novel stress-related peptide derived from the signal sequence of hsp60. The resulting complexes cannot efficiently be recognized by inhibitory CD94/NKG2A receptors. This was shown by lack of binding of HLA-E/hsp60sp tetramers to CD94/NKG2A expressing cells and by NK cell mediated killing of cells expressing such HLA-E/peptide complexes. Furthermore, the studies based on transfected cells suggest that hsp60sp can gain access to HLA-E molecules in vivo, particularly during conditions of cellular stress. It is therefore indicated that the proportion of HLA-E in complex with this peptide is increased during stress, leading to a gradual shift in the HLA-E peptide repertoire from NK cell protective to non-protective complexes.

According to this model, NK cells can detect stressed cells during infectious and inflammatory responses, through surveillance of HLA-E/peptide complexes in a peptide selective manner. This could be of particular importance for the subset of NK cells uniformly expressing CD94/NKG2A as their main inhibitory receptor, and also for the subset of activated T-cells that expresses this receptor.

It has previously been discussed whether missing-self recognition could be based on peptide specific recognition, in the sense that normal self peptides in complex with MHC class I would be permissive for binding of inhibitory receptors, while viral and other non-self peptides would be non-permissive. There is good evidence that some receptors are strongly influenced by the bound peptide. This applies to immunoglobulin-like as well C-type lectin-like receptors, including CD94/NKG2A. However, the protective capacity does not correlate with the origin of the peptide, i.e., whether it represents self versus non-self, or healthy versus sick. The balance between different HLA-E complexes may however represent a situation where cells can signal “normal” versus “abnormal” via peptides competing for MHC dependent presentation. The HLA-E mediated protection would thus not only rely on whether sufficient permissive signal peptides (mainly from various MHC class I molecules) are produced, but also on how these are balanced by non-permissive, stress induced peptides.

Although KIR recognition of MHC class I can be influenced by the bound peptides, a mechanism based on peptide selective surveillance of stressed cells may be primarily associated with the CD94/NKG2 receptors, as these are specifically designed to recognize the oligomorphic HLA-E molecules in complex with a restricted set of protective peptides. The KIRs, on the other hand, have primarily evolved to recognize a highly diverse repertoire of polymorphic HLA-A, -B, and -C molecules. A similar surveillance mechanism of stressed cells, if operating via KIRs, would require the presence of a vast array of stress-induced peptides capable of being loaded onto each HLA class I allele.

A first focus of investigation hereion with respect to Stress induced Peptide Interference (SPI) with inhibitory recognition relates to the structural aspects of different HLA-E peptide complexes. The crystal structure of HLA-E/B7sp reveals that five peptide residues lie within well-defined pockets of the HLA-E molecule (O'Callaghan et al., Mol. Cell. 1:531, 1998, incorporated herein by reference), constraining the conformation of the peptide throughout the binding groove. Comparison between hsp60sp and MHC class I signal peptide sequences (Table I) reveals differences at five positions: p1, p3, p5, p6, and p7. Of these, p3, p6 and p7 are buried in pockets D, C and E, respectively, while p1 and p5 are exposed to the surface.

Based on the HLA-E/B7sp structure, O'Callaghan et al. proposed that p5R in B7sp acts as an HLA-E contact residue for an HLA-E binding receptor. Indeed, a change in B7sp from arginine to valine (corresponding residue in the hsp60sp) at position 5 was sufficient to completely abrogate HLA-E mediated protection from killing by CD94/NKG2A expressing NK cells. However, the reciprocal change in hsp60sp (valine to arginine at p5) was not sufficient to gain protection, suggesting that additional amino acids in this peptide are important. Particular attention is focused here on the arginines at positions 3 and 7, which appear difficult to fit in the shallow and hydrophobic D- and E-pockets. Altering positioning or identity of these side chains is projected to interfere with receptor binding, either directly or indirectly by changing the overall conformation of the peptide in the HLA-E groove, and will therefore be useful within certain aspects of the invention.

Another important focus for further development within the invention concerns the biological relevance of HLA-E/hsp60sp complexes. The evidence presented above indicates that the increase of HLA-E levels observed during stress results from an influx of hsp60 derived peptides into the HLA-E presentation pathway. To critically investigate this K562 cells were co-transfected with HLA-E*01033 and the full-length hsp60 signal sequence coupled to GFP (hsp60L-GFP). This resulted in mitochondrial expression of GFP, while HLA-E was expressed at high levels intracellularly but only at low levels at the cell surface. The cell surface HLA-E levels were increased in such cells when they were subjected to culture induced stress, as compared to controls transfected with HLA-E*01033 and a mutated hsp60L-GFP construct where a critical HLA-E anchor residue had been substituted. Furthermore, up-regulation of HLA-E is also projected as a consequence of higher levels and altered distribution of endogenous hsp60sp during stress. In line with this, K562 cells transfected with HLA-E*01033 alone also displayed increased levels of cell surface HLA-E upon stress. Moreover, the up-regulation of HLA-E at the cell surface, as a result of stress, did not protect from NK cell mediated killing in any of these experiments.

HLA-E mediated protection may be regained, however, by adding a protective peptide, e.g. the B7sp peptide. Indeed, stressed cells were protected simply by adding the protective B7sp peptide in the assay. This indicates that endogenous hsp60sp can be presented by HLA-E during stress. Therefore, HLA-E is believed to be important as a presenter of stress induced peptides for NK cells and T cells during infection, autoimmunity, and inflammation. The elution and sequencing of peptides from isolated HLA-E molecules of cells growing under normal conditions and cells exposed to various stress stimuli will be evaluated to assess whether hsp60sp is indeed predominantly presented by stressed cells in these and other disease states and conditions.

The above results further indicate that at least a part of the stress-induced increased accessibility of hsp60sp to HLA-E must be due to post-transcriptional factors. Such factors could involve changes in protease activities, a more efficient peptide transport from mitochondria to the ER, an altered distribution of hsp60, or changes in the permeability of the mitochondrial membrane. A majority (80-90%) of the hsp60 pool is localized in the mitochondrial matrix in healthy cells (Soltys et al., Exp. Cell. Res. 222:16, 1996, incorporated herein by reference). There are however reports on increased levels of extra-mitochondrial hsp60 after bacterial infection (Belles et al., Infect. Immun. 67:4191, 1999, incorporated herein by reference) as well as after cellular stress and pro-apoptotic events (Feng et al., Blood 97:3505, 2001; Samali et al., Embo J. 18:2040, 1999, each incorporated herein by reference). These observations have been made with the mature hsp60, but at least the effects on mitochondrial permeability would also apply to the cleaved signal peptide.

In addition to altered mechanisms of peptide loading and increased expression of hsp60sp, other peptides capable of binding HLA-E may be up-regulated during stress. Even brief heat treatment of L-cells reportedly increases the cell surface levels of Qa-1^{b} (Imani et al., Proc. Natl. Acad. Sci. USA 88:10475, 1991, incorporated herein by reference). Recently it has been reported that an hsp60-derived peptide (GMQFDRGYL (SEC ID NO: 16) in Salmonella, and GMKFDRGYI (SEC ID NO: 11) in mouse) binds to Qa-1^{b}. (Lo et al., Nature Med. 6:215, 2000, incorporated herein by reference). In addition, the studies undertaken here confirm that the peptide GMKFDRGYI (SEC ID NO: 11) (hsp60.4 in table I) also can bind to HLA-E. In contrast to the hsp60sp, this peptide could not compete with the B7sp for binding to HLA-E (FIG. 5, panel d), nor could it reportedly compete for binding to Qa-1^{b} (Gays et al. J. Immunol. 166:1601-1610, 2001, incorporated herein by reference). It has also been reported that Qa-1^{b} presenting hsp60.4 fail to protect the cells from NK cell mediated lysis (Id.) These ligands thus appear incapable of engaging CD94/NKG2A receptors, but can instead be detected by clonotypic T cell receptors during Salmonella infection in mice (Lo et al., 2000, supra). It is therefore likely that peptides derived from other stress induced and heat shock proteins, including additional hsp60-derived peptides, may become HLA-E accessible during cellular stress provoked by an intracellular infection. These peptides are proposed to divert the functional role of the HLA-E molecules as ligands for CD94/NKG2 receptors towards complexes being able to be recognized by certain T cells via their antigen-specific TCR during an infection.

It should be noted that T cells can also express CD94/NKG2A inhibitory receptors, and the balance between HLA-E molecules with hsp60sp and MHC class I signal peptide is therefore also proposed to modulate T cells in inflammatory responses. In this context, the present findings are supported by a recently published report that effector cytotoxic T-lymphocytes directed against viral antigens may become restrained through expression of CD94/NKG2A (Moser, J. M. et al. Nature Immunol. 3:189-196, 2002, incorporated herein by reference). Recognition of Qa-1^{b} via this receptor inhibited proliferation and effector function of the T-cells, with a dramatic influence on acute infection as well as oncogenesis by polyoma virus. The authors speculated that the peptide loading of Qa-1^{b} could be affected under pathological conditions, possibly influencing the interaction with restrained T-cells.

The instant disclosure demonstrates loading of HLA-E with a peptide that is not only induced in stressed cells, but which also interferes with the protection against CD94/NKG2A⁺ NK cells normally conferred by HLA-E. These findings clarify the role of CD94/NKG2A expression during the regulation of T-cells responses. The co-expression of this receptor may complement the TCR pathway in the discrimination between healthy and sick cells, not only by sensing reduced production of MHC class I molecules but also increased accessibility to HLA-E of stress induced peptides. To further clarify these mechanisms, studies are comtempated to determine whether CD94[NKG2 expressing human T-cells can be influenced by stressed induced changes in target cells. In this context, analysis of peripheral blood from healthy donors verifies that the subset of CD94/NKG2A⁺ T-cells also binds to HLA-E/B7sp tetramers. Furthermore, additional studies herein showed revealed that no binding could be detected of HLA-E/hsp60sp tetramers to either CD94/NKG2A⁺ or CD94/NKG2A⁻ T cells, suggesting that T cells discriminate between different HLA-E complexes in the same way as NK cells, and that T cells expressing a TCR specific for HLA-E/hsp60sp are not abundant in healthy individuals.

HLA-E molecules are recognized by CD94/NKG2A inhibitory and CD94JNKG2C activating complexes. The role of the activating forms has not yet been clearly defined. The possibility that HLA-E/hsp60sp complexes are recognized by CD94/NKG2C or another, unknown activating NK receptor is appealing. This could explain why stressed K562-E*01033 cells were killed more efficiently by NK cells, despite the increased HLA-E levels. However, the NKL cell line does not express the activating NKG2C receptor, and HLA-E/hsp60sp tetramers did not bind to CD94/NKG2C transfectants, nor to any NK cells examined. Thus, other ligands that trigger NK cell activating receptors may be involved. Neither MIC-A, or MIC-B, ligands for NKG2D, are upregulated on culture stressed K562 or K562-E*01033 cells. However, additional ligands for NKG2D, or other activating receptors, may influence the sensitivity of K562 and K562-E*01033 cells. Further experiments using reagents that specifically block activating NK cell receptors may help to clarify the mechanism behind the increased NK cell sensitivity upon culture stress.

NK cells can be divided in two major subsets based on the level of CD56 cell surface expression (CD56^{dim} and CD56^{bright}) (Sedlmayr et al., Int. Arch. Allergy. Immunol. 110:308, 1996, incorporated herein by reference). Cells belonging to the minor CD56^{bright} subset all express high levels of CD94/NKG2A, and only a small fraction express KIRs. In contrast, most CD56^{dim} NK cells express KIRs and display a lower cell surface level of CD94/NKG2A (Jacobs et al., Eur. J. Immunol. 31:3121, 2001, incorporated herein by reference). The phenotypical division between CD56^{dim} and CD56^{bright} NK cells is associated with different effector functions (Cooper et al., Blood 97:3146, 2001, incorporated herein by reference). When stimulated, CD56^{bright} NK cells are less cytototoxic, and more prone to cytokine production and have therefore been proposed to be immunoregulatory (Chen et al., J. Immunol. 162:3212, 1999, incorporated herein by reference). These cells are potentially responsive to pro-inflammatory signals (based on their expression profile of chemokine receptors and adhesion molecules), and are largely over-represented at sites of inflammation (see below). Moreover, macrophages have been reported to respond to human hsp60 with increased production of IL-12 and IL-15 (Id.) which are important activators of this NK cell subset. Based on the findings presented here, and on the fact that hsp60 is up-regulated during inflammation, it is predicted that binding of hsp60sp by HLA-E mainly results in cytokine production by CD94/NKG2A⁺, CD56^{bright} NK-cells.

In the following Examples, additional findings are presented that include a showing that CD56-bright natural killer cells expressing a functional HLA-E specific inhibitory receptor are preferentially accumulated in the arthritic joint. As noted above, Natural killer (NK) cells are lymphocytes involved in the innate immune response against certain microbial and parasitic infections. Recent reports suggest additional important roles for NK cells in experimental autoimmune models, but little is yet known about the function of NK cells during autoimmune disease in man. In the following Examples, the expression of killer cell immunoglobulin (Ig)-like (KIR) and C-type lectin-like (CD94/NKG2) receptors specific for MHC class I molecules on NK cells, as well as on alpha/beta T cells and gamma/delta T cells derived from synovial fluid (SF) and peripheral blood (PB) of patients with arthritis, mainly rheumatoid arthritis (RA) is analyzed.

From these studies it is determined that the SF of arthritic patients contains an increased proportion of NK cells as compared to paired PB. In contrast to PB-NK cells, the SF-NK cell population almost uniformly expressed the CD94/NKG2A cell surface receptor and contained drastically reduced proportions of KIR⁺ NK cells. Functional analysis revealed that both in vitro cultured polyclonal SF-NK cells and PB-NK cells from patients are fully capable of killing a range of target cells. SF-NK cell cytolysis was, however, inhibitied by the presence of HLA-E on transfected target cells. When blocking CD94 on the SF-NK cells or by masking HLA on autologous cells, the SF-NK cells were capable to perform self-directed lysis. Thus, HLA-E is considered to play a fundamental role in the regulation of a major NK cell population in the inflamed joint.

Patients, Controls and Cell Separation

All 17 patients suffered from arthritis of the knee and had received therapeutic aspiration of SF at the time of analysis. RA patients fulfilled the American College of Rheumatology classification criteria for RA (Arnett, Arthritis Rheum. 31:315-324, 1988, incorporated herein by reference). All patients, except one 44 yr old female diagnosed with early oligoarthritis, received disease-modifying antirheumatic drugs. Extra-articular manifestations among the RA patients included diabetes mellitus (1 patient), Raynaud's phenomenon (2 patients), and secondary Sjögren's syndrome (1 patient). Paired samples of SF and PB from patients, and PB from 8 healthy female controls (mean age 52.7 yrs, range 49-61 yrs) were collected into preservative-free heparin and mononuclear cells were isolated by FICOLL-HYPAQUE (Amersham Pharmacia Biotech, Uppsala, Sweden) density gradient centrifugation according to standard protocols.

NK Cell Cultures

Generation of PB- and SF-NK cell lines was performed by depletion of CD3⁺ cells using anti-CD3 mAb (OKT3, American Type Culture Collection, Rockville, Md.) and pan anti-mouse Ig-coated dynabeads (bead to cell ratio of 4:1) as recommended by the manufacturer (Dynal AS, Oslo, Norway). The remaining NK cell enriched populations were maintained essentially as described previously (Söderström et al., J. Immunol. 159: 1072-1075, 1997, incorporated herein by reference), with minor modifications. Briefly, CD3⁻ cells were plated into a 24 well culture plate (Costar, Cambridge, Mass.) at a concentration of 1×10⁶ cells/ml in IMDM (Life Technologies, Gaithersburg, Md.) supplemented with 2% pooled human AB⁺ serum, 10% FCS, 2 mM L-glutamine, 100 U/ml penicillin, 100 μg/ml streptomycin and 100 U/ml of human recombinant IL-2. The CD56⁺CD3⁻ PB- and SF-NK cell lines were tested in functional assays two to three weeks after the initiation of the cultures.

mAbs and Flow Cytometry

Anti-KIR mAbs DX9 (anti-KIR3DL1), DX27 (anti-KIR2DL2, KIR2DL3 and KIR2DS2), DX31 (anti-KIR3DL2), and DX22 (anti-CD94/NKG2A, -B, and -C) were provided by Drs. Lewis L. Lanier and Joseph H. Phillips (UCSF, San Francisco and DNAX, Palo Alto, Calif. respectively). Other antibodies were against NKG2A (Z199, provided by Dr Lorenzo Moretta, Istituto Nazionale per la Ricerca sul Cancro, Genova, Italy), CD3 (UCHT1, BD Pharmingen, San Diego, Calif.), CD56 (B159, BD Pharmingen), CD16 (Leu-11c, Becton&Dickinson, San Jose, Calif.) TCRαβ (WT31, Becton&Dickinson) and TCRγδ (Immu 510, Coulter-Immunotech, Miami, Fla.), MHC class I (w6/32, American Type Culture Collection). Second-step reagents were FITC- and PE-conjugated rabbit anti-mouse Ig (both from Dakopatts, Glostrup,Denmark) and negative control for triple-colour (DAK-GO1, Dakopatts). lmmunofluorescenct staining was done using standard protocols. Cells were analyzed on a FACScan™.

Cells

K562 (human HLA-class I-erythroleukemia ), Daudi (human □2m-Burkitt's lymphoma), P815 (murine mastocytoma), 721.221 (human HLA-class I-B-lymphoblastoid cells) were maintained in complete medium consisting of RPMI 1640 (Life Technologies) supplemented with 10% heat-inactivated FCS, 2 mM L-glutamine, 100 U/ml penicillin, and 100 μg/ml streptomycin. HLA-B*5801 transfected 721.221 cells and 721.221 cells transfected with a chimeric gene composed of the HLA-G leader fused to HLA-B*5801 were produced at DNAX (Palo Alto, USA). Briefly, a chimeric cDNA containing the leader segment of HLA-G and the extracellular, transmembrane, and cytoplasmic domains of HLA-B*5801 was generated by PCR using wild-type HLA-G and HLA-B*5801 cDNA as templates (for details and primer sequences see Braud et al., Nature 391:795-799, 1991, incorporated herein by reference). The product was inserted into pBJ-neo expression vector and verified by sequencing. 721.221 cells were transfected by electroporation and selected in complete medium supplemented with 1 mg/ml G418. Transfected cells expressing high levels of cell surface HLA class I were isolated by flow cytometry. EBV transformed B-lymphoblastoid cell lines (B-LCL) were established by in vitro infection of patient B-cells with the B95-8 EBV-strain. Briefly, 10⁶ mononuclear cells from patient PB were incubated with the supernatant of a B95-8 EBV-producing cell line (Miller et al., Proc. Natl. Acad. Sci. USA 70:190-194, 1973, inch) for 1 hr in 5% CO₂ at 37° C. Infected B-cells were then cultured in complete medium supplemented with cyclosporin A (Sigma, St Louis, Mo.) at 5 μg/ml for approximately two weeks. Successful transformation was recognized by cell clumping and proliferation typical of in vitro established EBV-transformed B cells.

NK Cell-Mediated Cytotoxicity Assays

NK cell-mediated cytotoxicity was measured using a 4 hrs ⁵¹Cr radioisotope or a 18-hrs Alamar blue viability assay (Alamar Biosciences, Sacramento, Calif.) as previously described (Söderström et al., J. Immunol. 159: 1072-1075, 1997, incorporated herein by reference). In some experiments blocking mAbs at a final concentration of 1 μg/ml were added and present during the assay.

HLA-E Tetramer Production

The HLA-E expression vector for tetramer production was provided by Dr Veronique Braud (Oxford, UK). Tetrameric HLA-E complexes were generated essentially as described previously (Braud et al., Nature 391:795-799, 1991, incorporated herein by reference). Briefly, HLA-E heavy chain, fused with a BirA substrate peptide (bsp) at the c-terminus, and human beta-2 microglobulin (β2m) were overexpressed in E. coli BL21 pLysS, purified from inclusion bodies and solubilized in a 8M urea solution containing DTT. Complexes of HLA-E-bsp, human β2m and synthetic peptide (VMAPRTVLL (SEC ID NO: 11), derived from the HLA-B*0701 leader sequence, Research Genetics, Huntsville, Ala.) were produced by in vitro refolding of the HLA-E-bsp, human β2m and peptide. Refolded complexes were purified by size exclusion chromatography on a Superose 12 column (Amersham Pharmacia Biotech), and subsequently biotinylated using BirA enzyme (Avidity, Denver, Colo.) following the manufacturers' instructions. Free biotin was removed using NAP-5 desalting columns (Amersham Pharmacia Biotech). The degree of biotinylation was approximately 90%, as assesed by a gel-shift assay. Tetramers were generated by mixing biotinylated HLA-E/β2m/peptide monomers with streptavidin-PE (Sigma) at a 4:1 molar ratio.

Statistics

Percentages of positive cells are shown as mean±SEM. The paired student's T-test was used for comparisons between SF and PB.

### EXAMPLE V

Expression of KIR and CD94/NKG2 Molecules on NK Cells from Patients with Arthritis

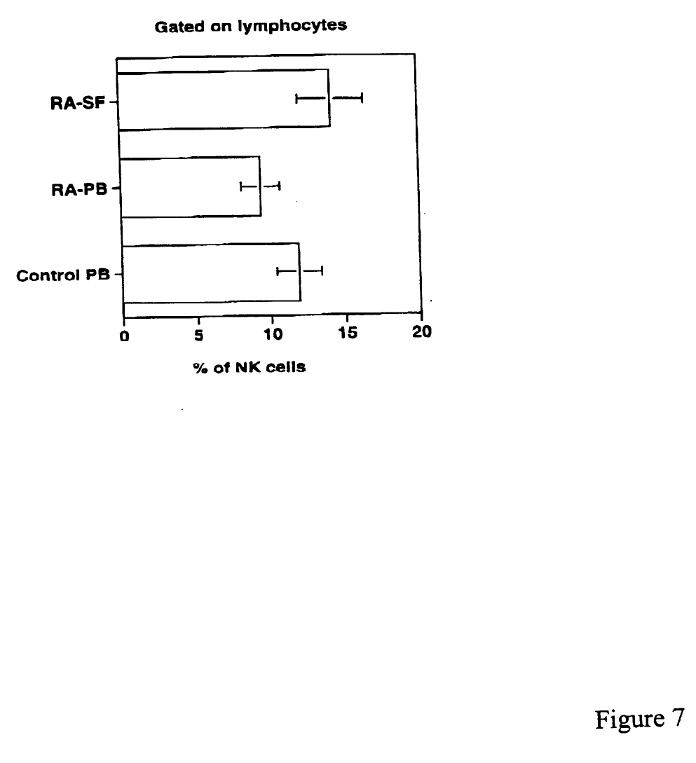

To determine the phenotype and subset distribution of freshly isolated NK cells derived from patients with arthritis, we performed triple stainings using a panel of mabs followed by flow cytometric analysis. As shown in FIG. 7, a slightly increased proportion of NK cells (CD3⁻CD56⁺) in SF as compared to patient PB was observed. In addition, most PB-NK cells of both patients and healthy individuals expressed the NK cell marker CD16 (FC□RIII), whereas a decreased frequency of CD16⁺ NK cells was observed in the SF, confirming other reports (Hendrich et al., Arthritis Rheum. 34: 423-431, 1991, incorporated herein by reference). Moreover, a small proportion of T-cells in both SF and PB of patients and healthy controls were double-positive for CD56 and CD3, but no significant difference was observed between SF and PB of patients and controls.

Notably, all patients had a markedly lower fraction of KIR3DL1⁺, KIR2DL2/KIR2DL3⁺ and KIR3DL2⁺ NK cells in the SF as compared to paired PB samples (Table 6). The expression of these KIR molecules on PB-NK cells of patients was heterogenous and apparently not different from PB-NK cells of healthy controls. A dramatic reduction in the proportion of NK cells expressing K]IR molecules specific for certain classical HLA-A, -B, and -C molecules in the SF suggested that SF-NK cells may rely on other MHC class I-specific inhibitory receptors distinct from the KIR-type of molecules to control their effector functions. Considering these results, an analsysis was undertakin of the expression of the lectin-like MHC class I-specific receptor (i.e. the CD94/NKG2 receptor complex), of which the CD94 chain paired with NKG2A (-or its splice variant NKG2B) forms an inhibitory unit that specifically binds to the non-classical HLA-E molecule (Braud et al., Nature 391:795-799, 1991, incorporated herein by reference).

TABLE 5
Expression of KIR molecules on NK cells.

| | Percentage of NK cells expressing:^{a)} | | | | | |
|---|---|---|---|---|---|---|
| | KIR3DL1 | | KIR2DL2/3 | | KIR3DL2 | |
| Subject | SF | PB | SF | PB | SF | PB |
| RA 1 | 3.2 | 18.7 | 3.5 | 17.5 | 3.7 | 13.4 |
| RA 2 | 14.7 | 28.6 | 16.6 | 21.5 | 22.8 | 42.4 |
| RA 3 | 7.7 | 15.4 | 8.6 | 25.7 | 11.9 | 18.6 |
| RA 4 | 2.0 | 14.2 | 6.0 | 25.3 | 6.6 | 23.6 |
| RA 5 | 2.0 | 2.8 | 5.6 | 22.0 | 12.8 | 21.7 |
| RA 6 | 2.5 | 19.1 | 6.7 | 21.6 | 5.6 | 10.4 |
| RA 7 | 5.0/4.8 | 20.9 | 10.6/10.3 | 38.2 | 15.6/14.2 | 30.9 |
| RA 8 | 0.1 | 0.1 | 8.1 | 8.9 | 3.7 | 6.5 |
| RA 9 | 0.8/1.8 | 9.4 | 5.4/9.2 | 27.0 | 4.1/5.9 | 13.0 |
| Psor. A | 10.6 | 37.3 | 7.2 | 27.0 | 12.0 | 30.8 |
| AS | 2.0 | 36.2 | 4.3 | 46.5 | 9.0 | 30.4 |
| Mono. A | 1.1 | 6.4 | 7.2 | 27.2 | 3.0 | 2.2 |
| Poly. A | 1.0 | 9.3 | 5.9 | 23.6 | 6.7 | 28.3 |
| Oligo. A1 | 3.7 | 5.6 | 7.0 | 55.2 | 11.5 | 13.0 |
| Oligo. A2 | 0.8 | 8.0 | 6.7 | 33.8 | 8.0 | 31.0 |
| mean ± SEM | 3.8 ± 0.9 | 15.5 ± 3.0 | 8.4 ± 1.1 | 27.1 ± 3.3 | 9.2 ± 1.3 | 21.1 ± 2.9 |
| control PB (n = 8) | | 9.8 ± 2.3 | | 30.1 ± 4.9 | | 25.3 ± 4.6 |
| mean ± SEM | | | | | | |
| ^{a)}Freshly isolated cells from SF and PB of patients and healthy subjects (control PB) were triple-stained with mAbs against various MHC class I specific receptors (KIR3DL1, KIR2DL2/L3 and KIR3DL2) using FITC-conjugated goat anti-mouse antibodies as a second step followed by conjugated mAbs against CD3 (Cychrome), and CD56 (PE). The results are shown as paired data for each individual patient. The SF values | | | | | | |
| # presented for RA patients 7 and 9 correspond to right/left knee. The percentage of KIR expressing cells within the CD56⁺CD3⁻gated lymphocyte population are shown (5000-10000 events within this NK cell gate were aquired). Patient samples contained drastically lower proportions of NK cells expressing KIR in the SF when compared to paired PB samples (p < 0.001 for KIR3DL1, p < 0.001 for | | | | | | |
| # KIR2DL2/L3, p < 0.001 for KIR3DL2; paired Students T test). The KIR expression on PB-NK cells of patients was not different from PB-NK cells of healthy controls. | | | | | | |

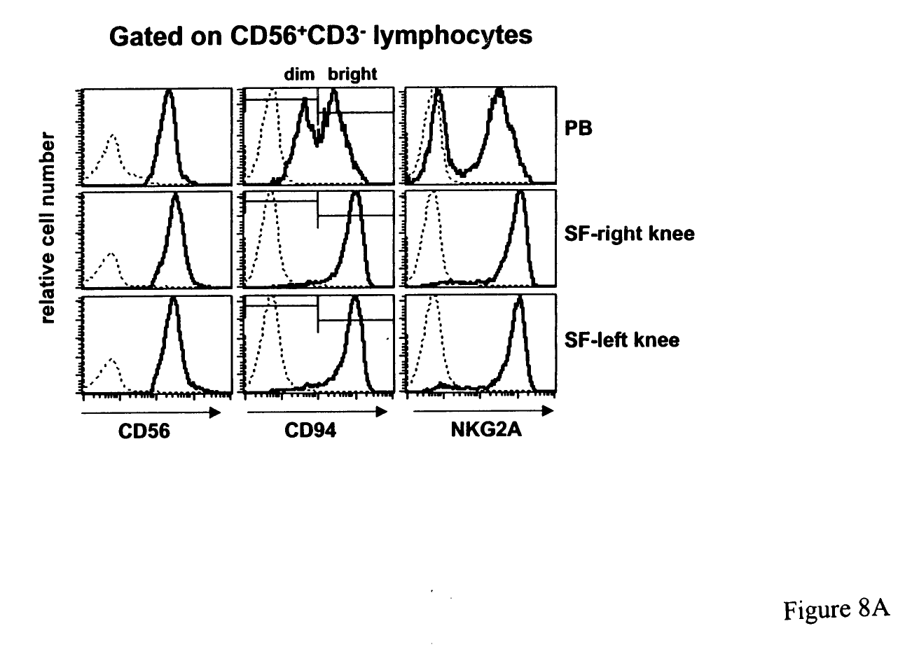

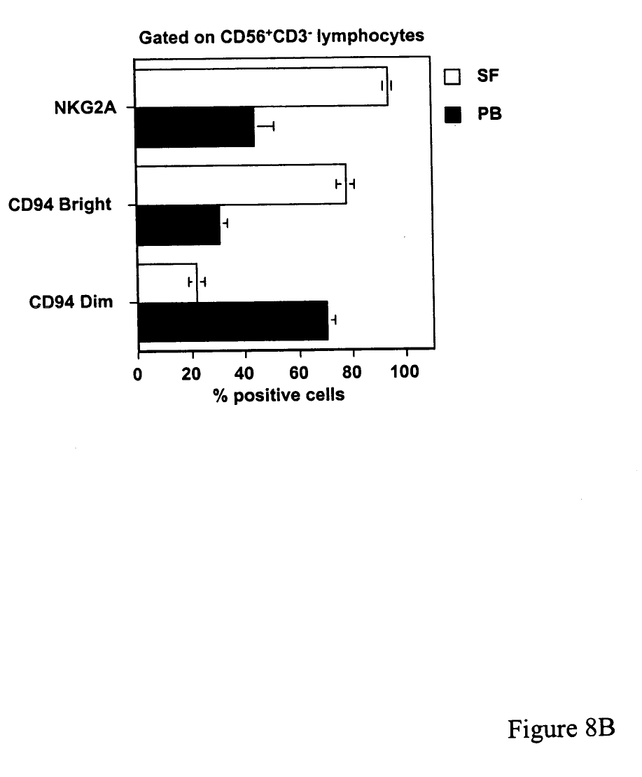

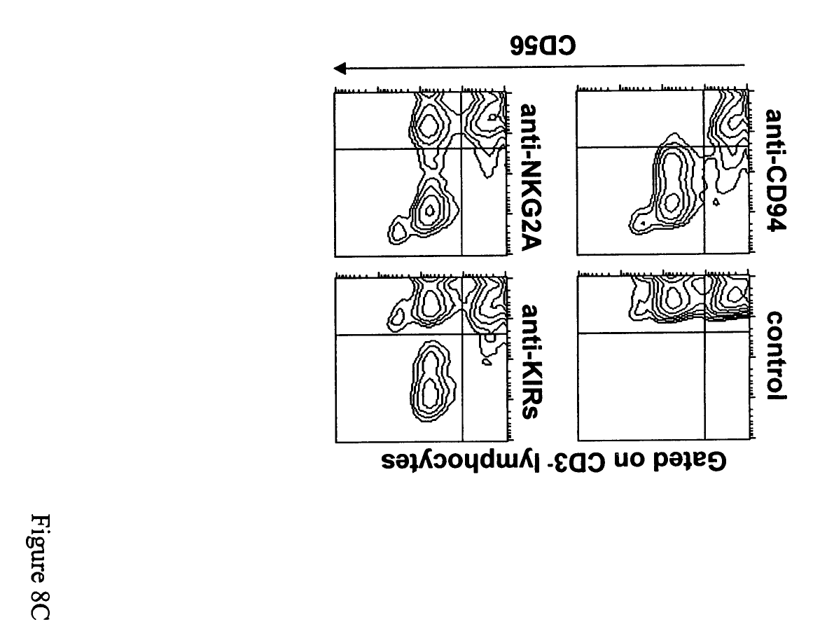

A clear, consistent finding was that most SF-NK cells stained brightly with an antibody against CD94 as a single histogram peak, whereas the anti-CD94 staining pattern on PB-NK cells was biphasic, dividing this population into a CD94^{dim} and a CD94^{bright} subset. FIG. 8A, shows the histogram profile of a representative patient, and FIG. 8B summarizes the data obtained from all patients studied). Interestingly, most SF-NK cells also brightly expressed the NKG2A molecule, whereas only a fraction of PB-NK cells were NKG2A⁺ (FIGS. 8A and 8B). These findings, together with the results presented in Table 6 demonstrate that the absolute majority of SF-NK cells express the inhibitory CD94/NKG2A receptor complex, and contain drastically reduced proportions of KIR expressing subsets. The expression levels of CD56 also demonstrate that the majority of SF-NK cells belong to a CD56^{bright} subset (57.7±5.3% n=16), whereas only a minor proportion of PB-NK cells express bright CD56 levels (24.1±5.0% n=14, p<0.001 compared to paired SF-NK cells), which is close to the proportion found among PB-NK cells from healthy subjects (17.5±3.2%, n=8).

These analyses further demonstrated that KIR expression on patient PB-NK cells was always confined to the CD56^{dim} subset, whereas the few CD56^{bright} PB-NK cells were CD94^{bright} and NKG2A⁺. Thus, based on the CD56, KIR, CD94 and NKG2A staining profiles, the predominant SF-NK cell subset resembles this minor subset of CD56^{bright} NK cells that is present in the PB of both patients as well as in healthy individuals (FIG. 8C), which similarly to SF-NK cells expresses the CD56, CD94 and NKG2A molecules at high levels, but seems to be almost completely devoid of at least KIR2DL2, KIR2DL3, KIR3DL1 and KIR3DL2 molecules. Taken together, the inflamed SF appears to be enriched for an NK cell subset with a more limited repertoire of MHC class I specific receptors as compared to the PB of patients and healthy controls.

### EXAMPLE VI

Expression of KIR and CD94/NKG2 on T-Lymphocyte Subsets in Patients with Chronic Arthritis

The expression of KIR and CD94/NKG2 molecules was measured on alpha/beta- and gamma/delta-T cells on patients and healthy subjects. Regardless if the cells were obtained from SF or PB, the fractions of KIR and CD94/NKG2A,B, and C expressing cells were lower among alpha/beta T cells when compared to gamma/delta T cells (Table 6 and Table 7, respectively). This was, however, not surprising since it is well-documented in the literature that these receptors are more common on gamma/delta T cells than on alpha/beta T cells. Interestingly, though, the fraction of alpha/beta T cells and gamma/delta T cells expressing some of these molecules was markedly different between paired PB and SF samples of some patients (Table 6, and Table 7, respectively), suggesting that a preferential accumulation of T cells expressing particular MHC class I specific receptors may occur in certain patients. The difference between SF and PB was more pronounced for gamma/delta T cells, but although some patients had a substantially lower proportion (>5 fold) of certain KIR⁺ subsets in the SF as compared to paired PB (see e.g. patients RA1, RA4, RA5, RA8 and Poly.A), a reverse pattern was also observed. Thus, the results did not reveal any clear trend suggesting that elevated, or reduced proportions of certain KIR and/or CD94/NKG2 expressing alpha/beta- or gamma/delta T cells are associated with either PB or SF of RA patients.

TABLE 6
Expression of KIR and CD94/NKG2A, B, C molecules on alpha/beta T cells.

| | Percentage of alpha/beta T cells exjressing:^{a)} | | | | | | | |
|---|---|---|---|---|---|---|---|---|
| | KIR3DL1 | | KIR2DL2/3 | | KIR3DL2 | | CD94/NKG2A, B, C | |
| Subject | SF | PB | SF | PB | SF | PB | SF | PB |
| RA 1 | 0.1 | 0.1 | 0.2 | 0.1 | 0.4 | 0.4 | 1.5 | 0.8 |
| RA 2 | 0.4 | 1.2 | 0.9 | 2.1 | 5.0 | 3.1 | 5.0 | 1.7 |
| RA 3 | 0.2 | 0.2 | 0.7 | 2.2 | 0.4 | 2.0 | 1.4 | 2.9 |
| RA 4 | 0.3 | 0.1 | 0.6 | 0.3 | 2.2 | 1.0 | 10.0 | 5.0 |
| RA5 | 2.2 | 0.9 | 4.4 | 3.1 | 3.5 | 2.0 | 12.1 | 10.6 |
| RA 6 | 0.5 | <0.1 | 0.4 | 11.6 | 1.0 | 0.3 | 4.8 | 0.1 |
| RA 7 | 0.2/0.3 | 0.7 | 1.3/1.6 | 1.5 | 3.4/4.0 | 3.0 | 1.9/3.0 | 4.9 |
| RA 8 | 0.1 | 0.3 | 0.2 | 2.0 | 1.8 | 1.4 | 7.3 | 18.2 |
| Psor. A | 0.3 | 0.6 | 0.6 | 1.9 | 1.1 | 1.1 | 1.5 | 4.4 |
| Mono. A | 0.2 | 0.3 | 3.3 | 6.3 | 3.4 | 2.3 | 5.4 | 15.2 |
| Poly. A | 0.3 | 0.1 | 1.0 | 1.0 | 2.5 | 0.4 | 6.5 | 8.0 |
| mean ± SEM | 0.4 ± 0.2 | 0.4 ± 0.1 | 1.3 ± 0.4 | 2.9 ± 1.0 | 2.4 ± 0.4 | 1.5 ± 0.3 | 4.8 ± 1.0 | 6.5 ± 1.8 |
| control PB (n = 8) | | 0.8 ± 0.4 | | 3.5 ± 0.6 | | 4.5 ± 1.0 | | 13.9 ± 3.2 |
| mean ± SEM | | | | | | | | |
| ^{a)}Freshly isolated cells from $F and PB of arthritic patients and healthy subjects (control PB) were triple-stained with mAbs against various MHC class I specific receptors (KIR3DL1, KIR2DL2/L3, KIR3DL2 and CD94/NKG2A, B, C) using FITC-conjugated goat anti-mouse antibodies as a second step followed by conjugated mAbs against CD3 (Cychrome), | | | | | | | | |
| # and TCRalpha/beta (PE). The results are shown as paired data for each individual patient. The results presented for patient RA 7 correspond to right/left knee. The percentage of KIR and CD94/NKG2 expressing cells within the CD3⁺TCRalpha/beta⁺ gated lymphocyte population are shown (5000-10000 events within the gate were acquired). | | | | | | | | |

TABLE 7
Expression of MR and CD94/NKG2A, B, C molecules on gamma/delta T cells.

| | Percentage of gamma/delta T cells expressing:^{a)} | | | | | | | |
|---|---|---|---|---|---|---|---|---|
| | KIR3DL1 | | KIR2DL2/3 | | KIR3DL2 | | CD94/NKG2A, B, C | |
| Patient | SF | PB | SF | PB | SF | PB | SF | PB |
| RA 1 | 1.4 | 21.8 | 1.2 | 3.2 | 14.1 | 16.0 | 80.2 | 89.5 |
| RA 2 | 1.4 | 4.0 | 6.2 | 22.1 | 41.2 | 43.4 | 91.5 | 80.9 |
| RA 3 | 6.5 | 24.5 | 32.5 | 23.8 | 56.0 | 32.0 | 78.1 | 86.0 |
| RA 4 | 1.0 | 1.7 | 2.4 | 12.3 | 16.0 | 13.8 | 92.8 | 75.4 |
| RA 5 | 1.4 | 9.3 | 20.0 | 28.2 | 27.7 | 19.6 | 73.5 | 88.6 |
| RA 6 | 4.3 | 2.4 | 3.1 | 11.0 | 16.4 | 14.4 | 70.6 | 62.1 |
| RA 7 | 1.3/1.7 | 8.3 | 8.4/10.9 | 17.6 | 41.7/39.9 | 31.5 | 54.4/53.2 | 65.1 |
| RA 8 | <0.1 | 2.1 | 8.2 | 16.6 | 13.0 | 14.8 | 55.3 | 66.3 |
| Psor. A | 2.3 | 1.6 | 3.8 | 1.5 | 13.5 | 2.2 | 58.6 | 92.4 |
| Mono. A | 0.8 | 0.8 | 8.0 | 12.8 | 13.8 | 10.8 | 81.0 | 87.0 |
| Poly. A | 0.2 | 17.8 | 1.5 | 0.1 | 7.4 | 27.6 | 94.4 | 90.3 |
| mean ± SEM | 1.8 ± 0.5 | 8.6 ± 2.6 | 8.6 ± 2.6 | 13.6 ± 2.8 | 25.0 ± 4.5 | 20.6 ± 3.6 | 72.8 ± 4.2 | 80.3 ± 3.4 |
| control PB (n = 8) | | 3.0 ± 0.4 | | 35.5 ± 0.6 | | 36.6 ± 1.0 | | 87.1 ± 8.3 |
| mean ± SEM | | | | | | | | |

^{a)}See Table 2 for specific details.

Functional Analysis of NK Cell Lines Derived from RA Patients

Paired polyclonal CD3⁻CD56⁺ SF-NK and PB-NK cell lines were established by in vitro cultivation in the presence of IL-2. After 1-2 weeks, the cytotoxic potential of these NK-cell lines was tested against a panel of target cells (721.221, K562, Daudi and P815). As shown in Table 5, both polyclonal SF- and ?B-NK cell lines were capable of lysing these different target cells. These NK-cell lines, as tested in Table 8 also produced comparable levels of the proinflammatory cytokines IFNgamma/delta, TNFgamma/delta, and IL-6, and also secreted similar amounts of IL-2 and IL-10 as measured in parallell using an ELISA assay, and more than 90% of the SF- and PB-NK cells stained for intracellular IFNgamma/delta after stimulation with PMA as measured by flow cytometry. Thus, no apparent difference with regard to the cytotoxic potential and cytokine production could be observed between in vitro established SF- and PB-NK cell lines.

TABLE 8
NK cell-mediated cytotoxicity against a panel of target cells using
polyclonal NK cell lines from SF and PB of a patient with RA

| Percentage of specific lysis^{a)} | | | |
|---|---|---|---|
| 721.221 | K562 | Daudi | P815 |

| E/T ratio | SF | PB | SF | PB | SF | PB | SF | PB |
|---|---|---|---|---|---|---|---|---|
| 4:1 | 64 | 76 | 54 | 57 | 76 | 66 | 38 | 51 |
| 2:1 | 40 | 69 | 41 | 52 | 51 | 54 | 16 | 32 |
| 1:1 | 20 | 34 | 25 | 30 | 38 | 46 | 3 | 7 |

^{a)}Lysis of target cells was detected using a 4 hrs ⁵¹Cr-release assay and data is shown at three E/T ratios using in vitro cultured polyclonal SF-NK cells (SF-left values) and PB-NK cells (PB-right values) derived from the same RA patient. The phenotype of the short term PB-NK cell line was heterogenous with regard to KIR and CD94/NKG2A expression, whereas the SF-NK cell line homogenously expressed CD94/NKG2A and essentially lacked expression of KIR.

### EXAMPLE VII

Synovial NK Cell Lines Functionally Recognize HLA-E Proposed as a Principal Ligand Protecting Autologous Cells from NK-Cell Attack

Surface expression of HLA-E depends on its binding to nonamer peptides derived from the signal sequence of some other HLA-A, -B, -C and -G molecules. Thus, the interaction of CD94/NKG2A with HLA-E can be regarded as a strategy by which certain NK cells indirectly monitor the expression of certain polymorphic and non-polymorphic HLA class I molecules. In transfection systems, overexpression of some HLA molecules (e.g. HLA-G which contain a permissive HLA-E binding signal sequence ) can assemble sufficient amounts of HLA-E to interact with CD94/NKG2A expressed on NK cells (Braud et al., Nature 391:795-799, 1991, incorporated herein by reference).

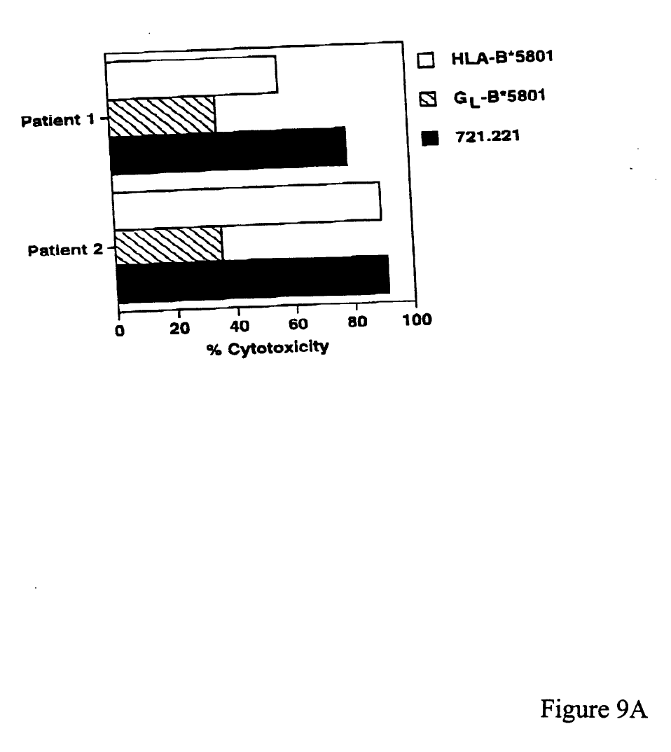

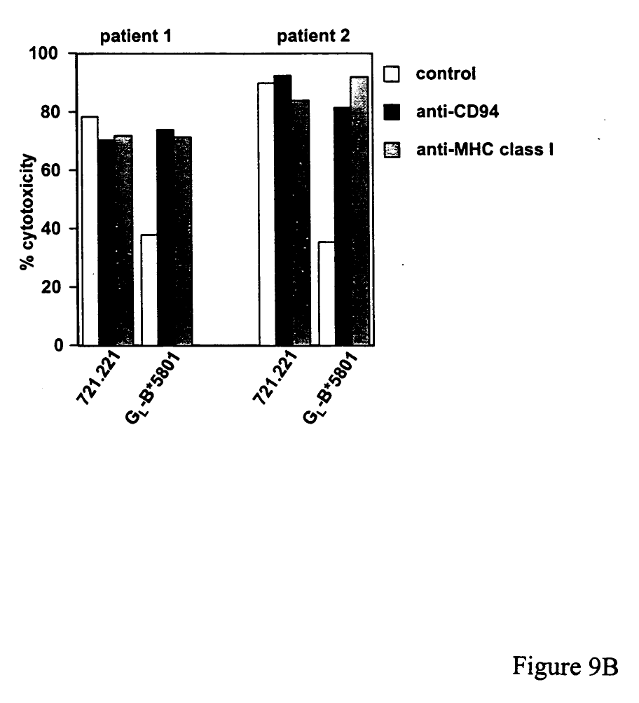

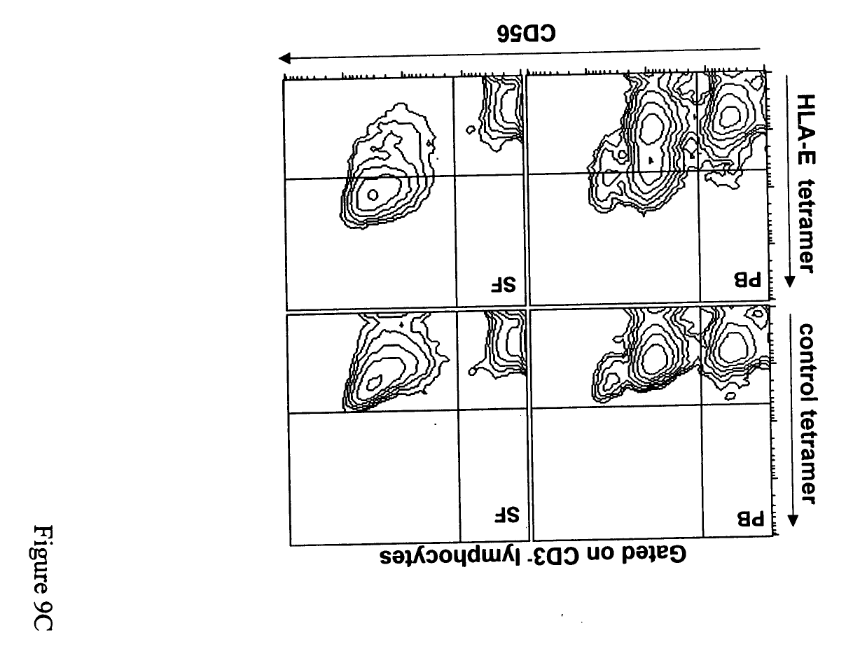

To test functionally whether SF-NK cells recognize HLA-E via their CD94/NKG2A receptor cytotoxic assays were conducted using 721.221 cells stably transfected with a chimeric gene in which the HLA-G leader sequence was grafted to the extracellular domains of HLA-B*5801 (G_{L}-B*5801). As a control 721.221 transfectant expressing the full-length HLA-B*5801 molecule (a HLA molecule that is not implicated in recognition by CD94/NKG2A receptors; see Phillips et al., Immunity 5:163-172, 1996 incorporated herein by reference) were employed. Both of these transfectants express HLA-B*5801 on the cell surface which is recognized equally well by KIR3DL1⁺ (and CD94/NKG2A−) NK cell clones previously shown to be a receptor specific for HLA-Bw4 type of alleles (Litwin et al., J. Exp. Med. 180:537-543, 1994; D'Andrea et al., J. Immunol. 155:2306-2310, 1995, each incorporated herein by reference). In addition to HLA-B*5801, the G_{L}-B*5801 transfected cells also surface express HLA-E that can be functionally detected by CD94/NKG2A⁺ NK cell clones. As shown in FIG. 9A, polyclonal SF-NK cell lines efficiently killed untransfected 721.221 cells as well as 721.221 cells transfected with wild-type HLA-B*5801. However protection from NK cell-mediated lysis was conferred by expression of the chimeric G_{L}-B*5801 molecule. The protection was reversed in the presence of antibodies against either CD94 or HLA class I, clearly showing that polyclonal SF-NK cells are uniformly capable of recognizing HLA-E via their inhibitory CD94/NKG2A receptor (FIG. 9B). In addition, when staining freshly isolated NK cells from patient PB and SF with HLA-E tetrameric molecules, which were refolded in the presence of the HLA-B*0701 nonamer leader peptide sequence, most of the SF-NK cells were efficiently stained with HLA-E tetramers, while fresh PB-NK cells were poorly stained (although some, including most of the CD56^{bright} PB-NK cells were HLA-E tetramer-positive; FIG. 9C shows one representative patient). The reason why many NK cells in the PB are not stained with the HLA-E tetramers is most likely due to the lower levels of CD94/NKG2 molecules on the CD94^{dim} subset.

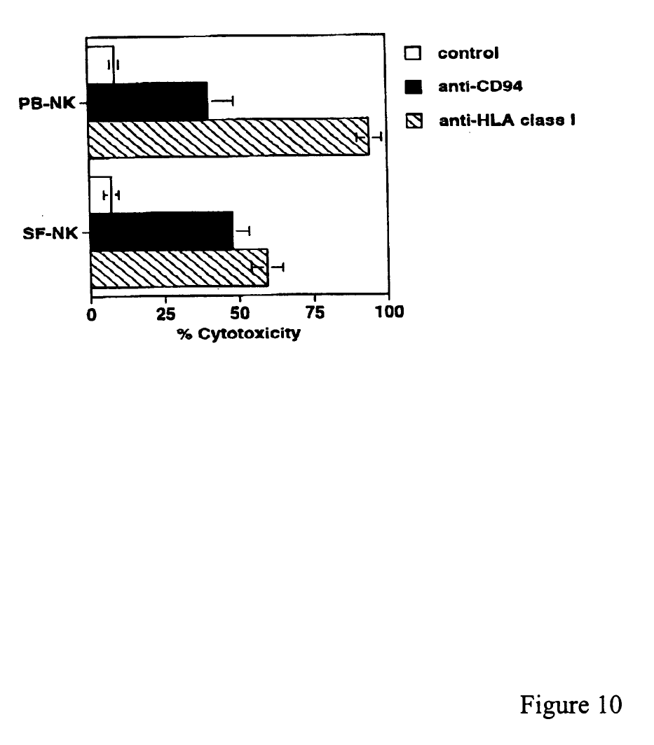

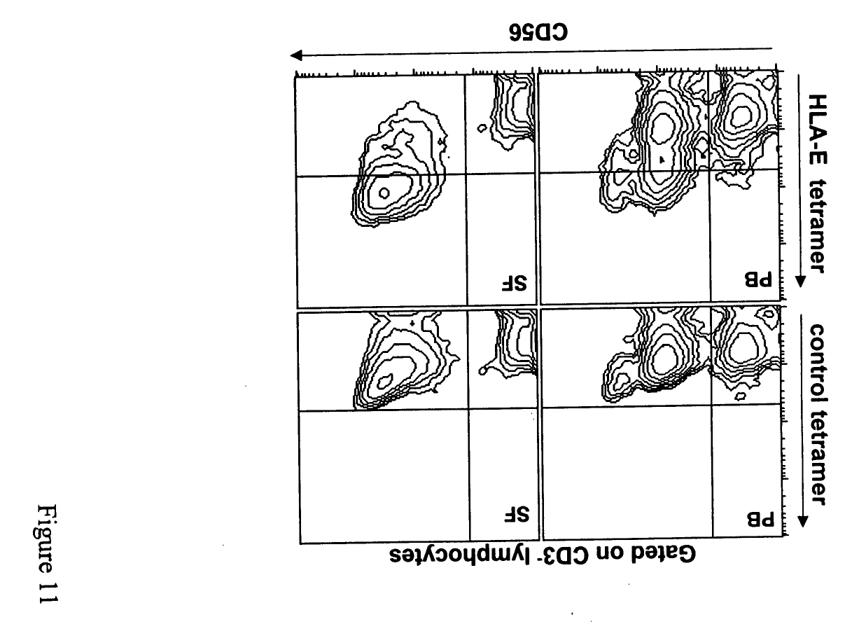

To test whether the interaction of CD94/NKG2A and HLA-E also may prevent SF-NK cells from killing autologous cells, B-LCL from one RA patient were used as targets in NK cell-mediated cytotoxicity. As shown in FIG. 10, both PB-NK cells and SF-NK cells were unable to lyse autologous B-LCL. Cytolysis of autologous cells was, however, augmented by an anti-HLA class I mAb or an anti-CD94 m-tAb using both polyclonal PB-NK cells and SF-NK cells as effectors. Using SF-NK cells as effectors the anti-CD94 mAb restored the lysis to almost the same level as observed with anti-HLA class I miAb—indicating that most of the self-HLA protective mechanism involves CD94/NKG2A interacting with HLA-E. On the other hand, the polyclonal PB-NK cell line was also regulated by other receptor-ligand interactions since addition of anti-CD94 only partially increased the lysis, whereas anti-HLA class I led to almost complete lysis of autologous target cells. These findings are in accordance with the fact that only a fraction of the polyclonal PB-NK cells were CD94/NKG2A⁺ while almost all SF-NK cells were CD94/NKG2A⁺. Moreover, the results from this patient, shown in FIG. 10, indicate that the CD94/NKG2A is the main self-specific receptor present on SF-NK cells.

Additional studies further clarify the important uses of the invention for modulating HLA-E/CD94/NKG2 cellular receptor interactions and related immune responses associated with RA and other inflammatory and autoimmune diseases, including adverse graft rejection responses. As noted above, human chronic joint inflammation is perpetuated by a cascade of inflammatory cytokines being produced in the synovial membrane and fluid. NK cells are potent producers of cytokines and are present at these inflammatory sites, but their role in chronic human arthritis was heretofore largely unknown. The function of NK cells is regulated by inhibitory and activating cell surface receptors interacting with molecules on neighbouring cells. In the present disclosure synovial fluid (SF) NK cell expression of killer immunoglobulin like receptors (KIRs) and the C-type lectin like receptor CD94/NKG2A was studied in detail. The ability of NK cells to produce proinflammatory cytokines IFN-gamma and TNF-alpha was also investigated in detail. The novel modulation of NK cell cytokine production (IFN-gamma and TNF-alpha) in the presence of target cells expressing inhibitory HLA-E+ peptide complexes is reported.

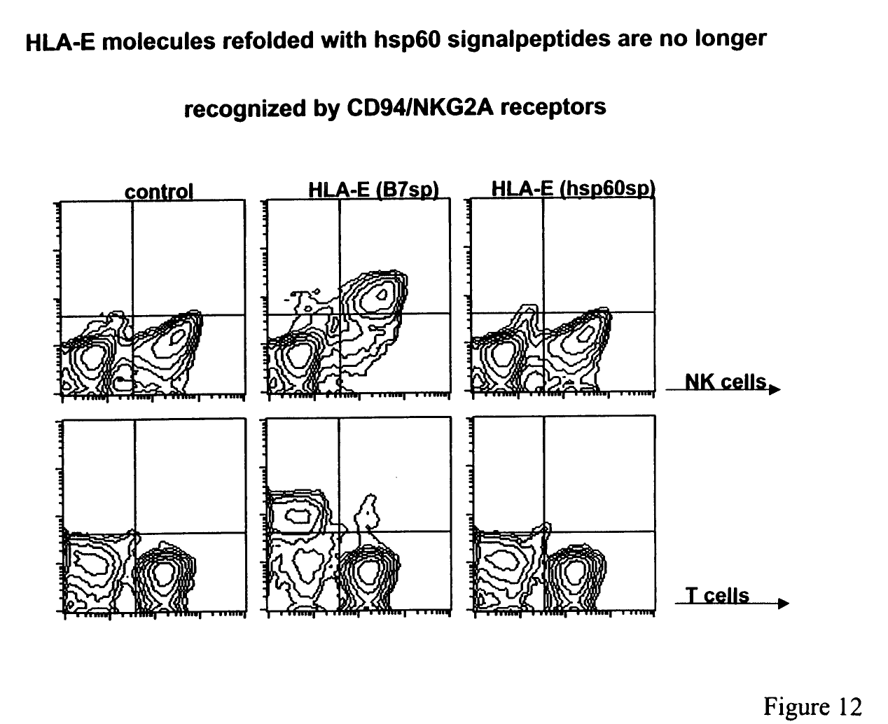

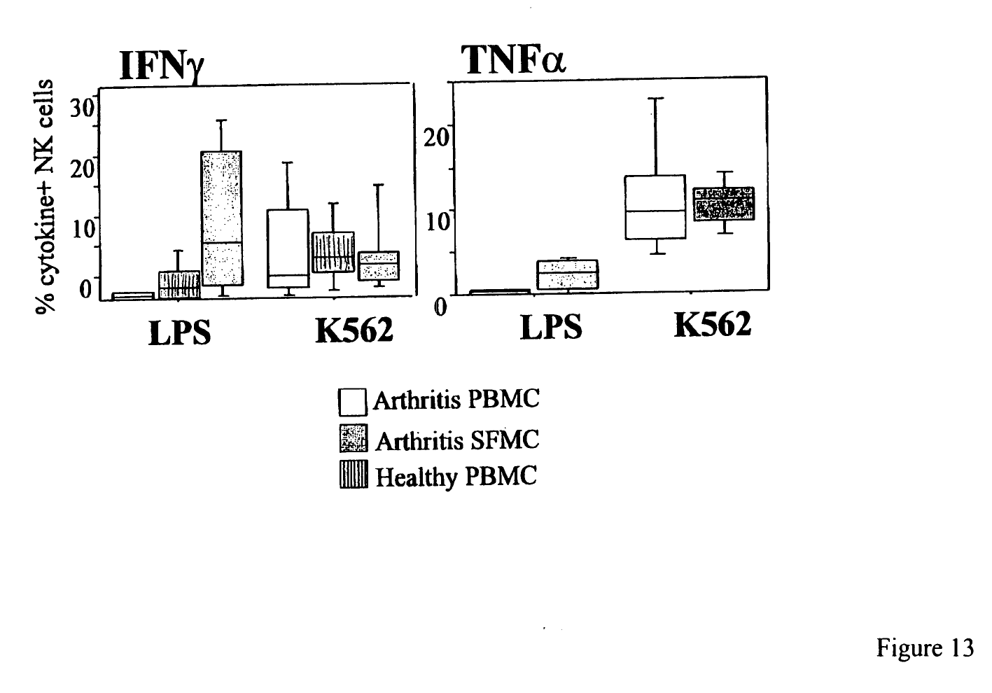

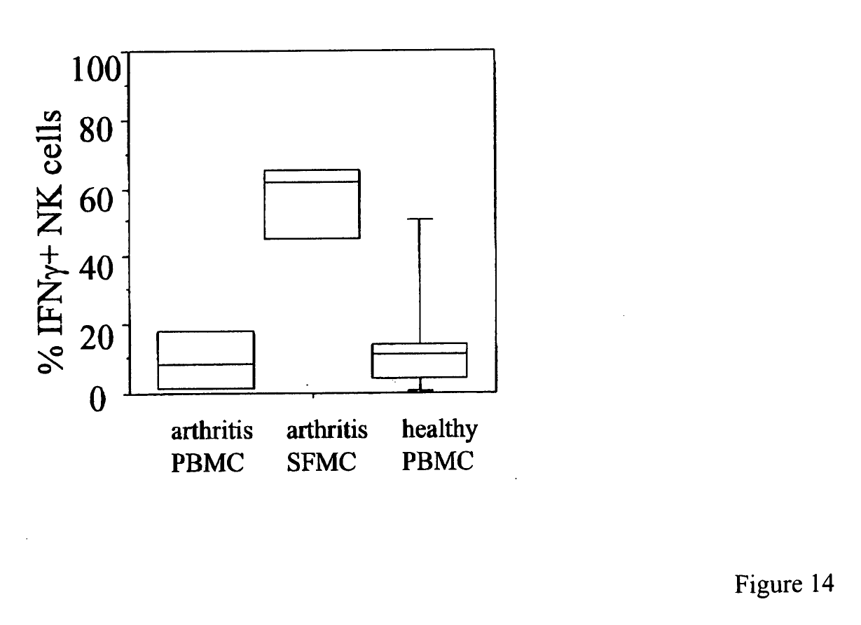

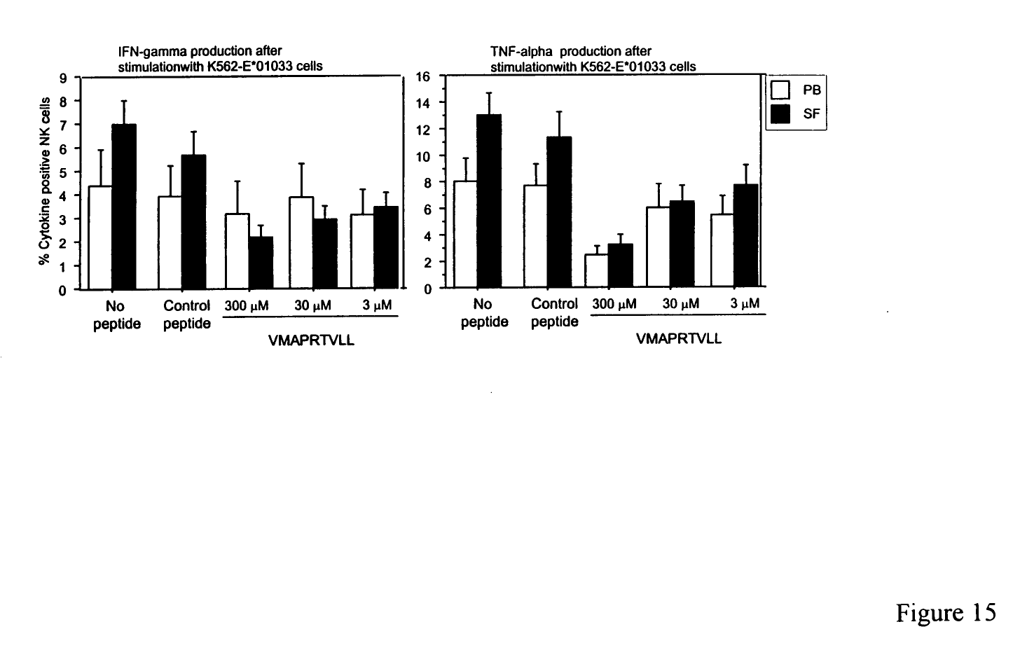

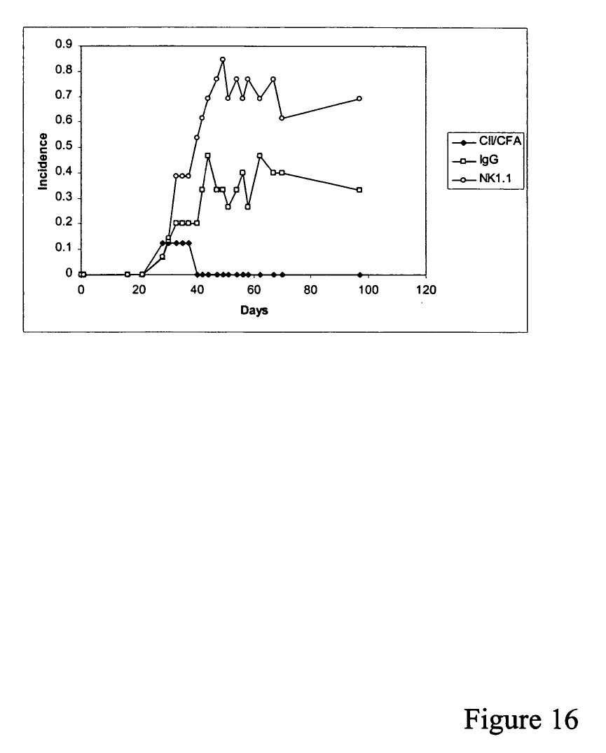

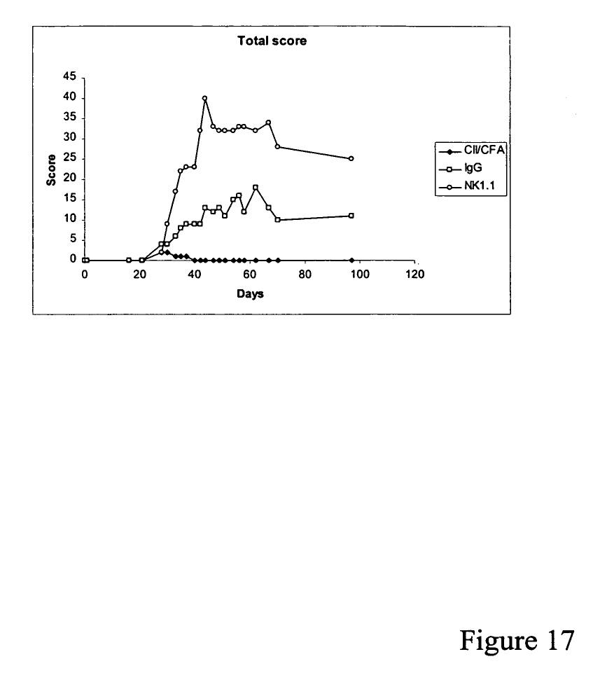

Supplemental to the foregoing Examples, FIG. 11 demonstrates that SF-NK cells bind to HLA-E in complex with an exemplary, VMAPRTVLL (SEC ID NO: 3) peptide. FIG. 12 shows that SF-NK cells bind to HLA-E in complex with VMAPRTVLL (SEC ID NO: 3) (B7sp) peptide but not to HLA-E in complex with QMRPVRSVL (SEC ID NO: 2) (hsp60sp) peptide. FIG. 13 demonstrates that SF-NK cells are stimulated to produce IFN-gamma and TNF-alpha upon exposure to lipopolysaccharide (LPS) as compared to PB-NK cells of either RA patients or healthy individuals. FIG. 14 shows that SF-NK cells are stimulated to produce IFN-gamma after exposure to IL-2 as compared to PB-NK cells. FIG. 15 demonstrates that HLA-E presenting B7 signal peptide (VMAPRTVLL) (SEC ID NO: 3) are sufficient to inhibit NK cell IFN-gamma and TNF-alpha cytokine production in these accepted model studies

The foregoing supplemental results show that human NK cells found in joint fluid from patients with chronic inflammatory arthritis belong to a phenotypically and functionally distinct subset of NK cells, similar to the earlier described CD56-bright peripheral blood NK cell subset. NK cells in the arthritic joint may add to the proinflammatory cascade by their potent production of IFN-gamma and TNF-alpha in response to other cytokines produced in the joint and these NK cell cytokine responses will be significantly down-modulated by cell contact with cells expressing HLA-E together with a protective peptide, a complex recognized by CD94/NKG2A inhibitory receptors. HLA-E in complex with a non-protective peptide, not recognized by CD94/NKG2A inhibitory receptors, is not capable of inhibiting NK cell cytokine production.

Summarizing the foregoing Examples, NK cells from SF of arthritic patients were found to phenotypically belong to a distinct subset of NK-cell, mainly lacking KIR molecules and homogenously expressing the inhibitory CD94/NKG2A heterodimer. The present disclosure is believed to be the first description of a unique disease-associated accumulation of a certain NK cell subset in any autoimmune disease in man.

Prior studies have established that KIR expression on normal PB-NK cells is clonally distributed and variably expressed among individuals (Lanier et al., Immunity 6:371-378, 1997, incorporated herein by reference). Moreover, both the surface levels of KIR and frequency of at least some KIR⁺ NK cell subsets in PB seem to be stable over time, and independent of the individual's HLA class I haplotype (Gumperz et al., J. Exp. Med. 183:1817-1827, 1996, incorporated herein by reference). Thus, NK cells expressing certain KIR isoforms may be present in individuals who lack the appropriate self-HLA class I molecule and can be absent in those who possess it (Id.) Therefore, certain individuals seem to possess NK cells that use either inhibitory KIRs or CD94/NKG2A for self recognition of MHC class I (Valiante et al., Immunity 7:739-751, 1997, incorporated herein by reference). Although some individuals rely on the more “broadly” reactive CD94/NKG2A system there is no apparent decrease in the expression of KIR on their PB-NK cells. Therefore, the apparently normal expression pattern of KIR3DL1, KIR2DL2,/KIR2DL3 and KIR3DL2 molecules on PB-NK cells of RA patients, and the specific accumulation of SF-NK cells of which the majority seem to lack expression of these KIR molecules and instead mainly express the CD94/NKG2A receptor, suggests that the inflamed joint provides an environment for only certain NK cell subsets.

The phenotypic similarities between the small subset of CD56^{bright} PB-NK cells and the major SF-NK cell subset, suggest that this minor PB subset is preferentially recruited to the inflamed joint in response to locally produced chemotactic factors, such as e.g. macrophage inflammatory protein-1α (MIP-1β, MIP-1α or RANTES, known to be present in the inflamed joint (Hosaka et al., Clin. Exp. Immunol. 97:451-457, 1994, incorporated herein by reference), and this projected mechanism will be evaluated to further refine the teachings herein. Based on a study showing that the CD56^{bright} PB-NK cell subset also expresses brighter levels of molecules important for leucocyte rolling on the vessel wall (e.g. CD62L) as well as molecules necessary for adhesion and extravasation of leucocytes into inflammatory sites (e.g. CD2, CD11c, CD44, CD49e, CD54)(26), it is likely that the CD56^{bright} CD94/NKG2A⁺ KIR⁻ NK cell subset is selectively recruited to the inflamed joint. In addition, cytokines (e.g. IL-15) present in the joint may promote preferential proliferation and/or be involved in the rescue from apoptosis of this particular subset.

In addition to the foregoing findings, the Examples herein demonstrate that SF-NK cells are functionally capable of recognizing HLA-E. Evidence is also provided that this recognition is the main functional receptor-ligand interaction preventing SF-NK cells from attacking autologous cells.

The presence of a uniformly expressed, broadly reactive system may be important for NK cells in the inflamed joint, since the CD94/NKG2A receptor itself indirectly recognizes the presence of a large fraction of HLA-class I molecules containing the permissive leader peptide (Braud et al., Nature 391:795-799, 1991, incorporated herein by reference). However, relying mainly on this receptor-ligand interaction may also render this system vulnerable, since self tolerance by SF-NK cells is maintained solely by HLA-E expression. Therefore, maintaining a high level of HLA-E expression on cells within the joint would be necessary in order to prevent SF-NK cell-mediated cytotoxicity. It may be presumed that as long as classical MHC class I molecules are produced at normal levels, sufficient amounts of protective leader-peptides would be generated for intracellular loading of HLA-E molecules and subsequent cell-surface localization to inhibit SF-NK cell responses. However, it has been reported that abberantly low levels of MHC class I expression are found on the cell surface of lymphocytes in patients with various autoimmune diseases, including RA (Fu et al., J. Clin. Invest. 91:2301-2307, 1993, incorporated herein by reference). It is proposed here that upon proper SF-NK-cell stimulation, these HLA-E levels would be sufficiently low, enough to induce NK cell responses after interaction with certain lymphocytes which could serve an important regulatory role in the synovial compartment. In this regard, it is interesting to note that patients with a genetic deficiency in the transporter associated to antigen processing (TAP), which consequently express no—or low amounts—of MHC class I cell surface molecules on all cells, overexpress a functional CD94/NKG2A receptor on their NK cells, suggesting that an adaptation to low levels of MHC class I is associated with the expression of this receptor (see, e.g., Zimmer et al., J. Exp. Med. 187:117-122, 1998, incorporated herein by reference). Furthermore, in vitro activated NK cells from these patients effectively lysed autologous LCL cells and fibroblasts, suggesting that tolerance of this subset could be broken, rendering these NK cells capable of autoimmune reactivity against cells expressing low levels of MHC class I (Id.)

Although many reports have shown that freshly isolated NK cells from RA patients generally show a reduced lytic activity (reviewed in Lipsky, Clin. Exp. Rheumatol. 4:303-305, 1982, incorporated herein by reference) and respond poorly with IFNα production when stimulated (Berg et al., Clin. Exp. Immunol. 1:174-182, 1999, incorporated herein by reference), other reports have shown that depletion of NK cells from SF mononuclear cell-cultures in vitro resulted in enhanced production of certain Ig-isotypes (Tovar et al., Arthritis Rheum. 29:1435-1439, 1986, incorporated herein by reference). This suggests that SF-NK cells are involved in the regulation of antibody production, which perhaps could be due to direct cytolysis of certain B cells or indirectly by cytokine production (e.g. TGFβ) which may in turn induce suppressive T cell responses (Horwitz et al., Immunol. Today 18:538-542, 1997, incorporated herein by reference.

Collectively, the foregoing results evince that the SF of patients with autoimmune arthritis contain a significantly increased fraction of NK cells of which the absolute majority express the CD94/NKG2A receptor and only a minority express KIR3DL 1, KIR3DL2/3 and KIR3DL2 molecules. Evidence is also provided that the SF-NK cells are capable of binding HLA-E, and that they functionally recognize HLA-E on transfected cells. Furthermore, the CD94/NKG2A receptor expressed on the polyclonal SF-NK cell line seems to be the main receptor involved in the regulation of self-MHC class I reactivity, as shown by using autologous LCL cells in blocking experiment.

### EXAMPLE VIII

Screening of Synthetic HLA-E Binding Peptides with “On/Off Switch” Capacity to Engage CD94-NKG2 Receptor-Pairs

HLA-E complexed with hsp60 leader peptide may be somewhat unstable and tend to dissociate when peptide loaded cells are transferred to 37° C. during NK cell cytotoxic assays. Identifying peptide variants that may enhance or reduce stability of these binding interactions will provide additional active agents for use within the methods and compositions of the invention, including for in vivo therapeutic uses. To refine these aspects of the invention, a large-scale screening of synthetic peptides or peptide analogues may be conducted using peptide variants characterized by subtle modification of the hsp60 peptide back-bone. This screening can be employed to isolate stable HLA-E binding peptides, which may show enhanced functional interaction with activating CD94/NKG2 receptor pairs. Isolation of such peptide analogues has future interest for therapy against a broad range of tumors. The following is a brief outline of an exemplary large-scale screening program to identify useful peptide variants within the invention.

Materials:

K5 62 cells transfected with either HLA-E*0101 or HLA-E*01033.

Comment: To speed up a large scale peptide-screening approach these cell lines could be cotransfected also with GFP-encoding plasmid (see below).
RPMI 1640 medium
Nonamer peptide library, modified peptides based on the hsp60 leader peptide back-bone, other potential synthetic or natural structural analogues binding to HLA-E peptide binding cleft.
26° C. incubator
37° C. incubator
Cell centrifuge with 96 well plate-holders
96 well round-bottomed plates
   Initial Screening for HLA-E Stabilization

Studies of peptide and HLA-E interactions have shown that the presence of an HLA-E binding peptide provided by the addition of a synthetic nonamer peptide in the culture medium, may sufficiently stabilize and upregulate HLA-E cell surface expression levels as measured by flow cytometry. To test whether synthetic peptides/peptide analogues bind HLA-E, we will stabilize HLA-E cell surface levels at 26° C. over-night using HLAE*01033 or HLA-E* 0101 transfected K562 cells.

Procedure to Screen Out HLA-E*0101 and HLA-E*01033 Binding Peptides/Peptide Analogues

HLA-E transfected K562 cells will be washed two times in RPMI medium without FCS and put up in 96 well round-bottomed plates at 2×10e5 cells/well in 200 microliter RPMI medium containing 300 microM peptide. Plates are incubated over night at 26° C., then washed two times in RPMI 1640 medium without FCS. An aliquot will be stained with anti-class I mAb and analyzed by flow cytometry for HLA class I expression levels. The remaining cells will be put back at 37° C. and stained 1, 2, 3, or 4 hrs, later to get an estimate of the stability of HLA-E peptide complexes. By this approach it will be able to screen out a panel of HLA-E binding peptides that will form a rather stable complex.

Procedure to Evaluate the Potential Function of HLA-E Peptide Complexes by their Potential to Engage either CD94/NKG2A (Inhibiting) or CD94/NKG2C (Activating) Receptor Pairs

Effector NK Cells to Use for Screening:

NKL and Nishi NK cell lines, which both bear inhibitory CD94/NKG2A receptor pairs, will be initially analyzed for the presence of NKG2C cDNA transcripts by RT-PCR or by the aid of a NKG2C specific mAb and cell surface staining followed by flow cytometry. If these cell lines are devoid of the activating NKG2C receptor chain, heterogenous polyclonal NK-cell populations established from the rheumatoid joint, which predominantly express a functional CD94/NKG2A receptor pair, will be analyzed for the presence of also the activating NKG2C receptor chain.

Procedure:

HLA-E transfected K562 cells, which have been stable co-transfected with GFP, will be loaded with our selected panel of HLA-E stabilizing peptides in 96 well plates, as detailed above. After washing, these target cell plates-will be incubated with effector NK cells 37° C. for 2-4 hrs, and NK-cell mediated cytotoxicity will be directly analyzed by flow cytometry without prior washing. The exact details and kinetic requirement for this assay procedure will be initially determined experimentally using exemplary HLA-B*0701 signal peptide (VMAPRTVLL) (SEC ID NO: 3) and hsp60 signal peptide (QMRPVRSVL) (SEC ID NO: 2) loaded HLA-E*0101 and HLA-E*01033 transfected GFP-positive K562 cells. The assay is based on that HLA-E transfected target cells that are protected from NK-cell mediated lysis will remain GFP-positive (green fluorescent) and stay in the viable gate, cells that are being lysed will loose green fluorescent and eventually end up outside the viable gate. The advantage of this assay is that one can quickly screen a rather large peptide library at once. A potential disadvantage may be that the threshold levels to determine whether a target cell is lysed at a significantly higher ratio as compared to control peptide-treated cells, may be difficult to assess. Therefore, it may be desirable to use this method initially to select away peptides or peptide analogs that show good protection from lysis. The effect of the remaining selected peptides is then tested by traditional methods (i.e. NK cell-mediated cytotoxicity using 51-Cr radioisotope labeled target cells). This experimental approach enables screening for novel peptides and peptide analogues that work as an “offswitch” for CD94/NKG2A inhibitory receptors, and potentially as an “on-switch” for CD94/NKG2 activating receptors. Finally, this experimental approach will enable screening for novel peptides and peptide analogs that form stable protective HLA-E complexes (i.e. “on-switch” for CD94/NKG2A inhibitory receptors).

### EXAMPLE IX

Demonstration that Qa-1^{b} (Murine Homologue of HLA-E) is Involved in Tumor Escape, and that Enhanced Rejection of Qa-1^{b} Expressing Tumors can be Achieved by the Administration of CD94-NKG2A Uncoupling Peptides.

HLA-E/hsp60 complexes are raised during cellular distress. This complex is not recognized by inhibitory CD94-NKG2A receptors. CD94-NKG2A is not only expressed on NK cells, but also on subsets of gamma/delta T cells and CD8+ cytotoxic T cells (CTLs). Downregulation of classical MHC class I molecules occurs in many tumors, possibly as an escape mechanism from immune detection. Therefore, e.g. a melanoma cell with downregulated classical MHC class I, but with retained expression of HLA-E, is probably a challenging target for the immune system, having inhibited both the CTL and the NK activity. By the use of hsp60 signal peptides or other proinflammatory HLA-E binding peptides (e.g., from stress proteins, heat shock proteins or other exemplary proteins disclosed herein), and analogs thereof, with strong capacity to bind HLA-E and which potentially can compete out protective MHC class I-peptides in the cleft of HLA-E, a novel therapeutic tool can be developed to induce the activation of NK cells and to lower the threshold for activation of CD94-NKG2A expressing CTLs against tumor cells that have escaped immune detection on the basis of retained protective HLAE expression.

Initially Qa-1b expressing tumor cells are loaded with a selected peptides or peptide analogues, and with hsp60 peptide (GMKFDRGYI (SEC ID NO: 11)—a known Qa-1b binding, CD94/NKG2A uncoupling peptide (see Lo et al., Nature Med. 6:215-218, 2000, incorporated herein by reference), as well as AMAPRTLLL (Qa-1b binding CD94/NKG2A coupling peptide (Kraft, J. Exp. Med. 192:613-623, 2000). Analyses will be conducted in NK-cell depleted (anti-NK1. I treated) and non-depleted mice to determine whether enhanced NK-cell dependent tumor rejection is observed by using CD94-NKG2A-uncoupling peptides, and likewise whether enhanced tumor establishment is observed using, CD94-NKG2A-coupling peptides.

### EXAMPLE XIII

Study of Murine NK Cells in Experimentally Induced Arthritis

In this model, the potential role for NK cells in the establishment and maintenance of collagen-induced arthritis (CIA) is determined. By using NK-cell depleting antibody (NK1.1) before induction of the disease and during established disease, the role of the presence and absence of NK cells in the pathogenesis of disease will be further clarified. Various tissues in NK cell depleted and non-depleted mice (e.g. spleen, lymph-nodes, blood, joint-tissue) are collected and analyzed for the presence of NK cells and their expression of various cell-surface markers. A principal goal of these analyses is to evaluate whether inflamed tissue in mouse CIA, like synovial fluid of human rheumatoid arthritis (RA), -accumulate certain unique subsets of NK cells expressing predominantly the CD94-NKG2A receptor pair specific for the HLA-E homolog in mice, termed Qa-1^{b}. These studies further clarify the role that non-classical HLA-E/Qa-1b play in regulating NK cells at inflammatory sites.

It has been shown that immunization with collagen type II results in collagen-induced arthritis in C57B1/6 mice, with joint histopathological changes similar to human RA (Cambell, et al. Eur. J. Immunol. 30:1568-1575, 2000). Importantly, the C57B1/6 mice carry the H-2b haplotype carrying the Qa1b-binding protective nonamer signal-peptide, termed qdm (AMAPRTLLL) and have NK cells that can be detected by the anti-NK1.1 antibody. This CIA model enables further clarification of the role of Qa1b+ peptide and its interaction with mouse CD94/NKG2 receptors expressed on NK cells and NK1.1-positive T cells.

Mice

C57BL/6 (H-2^{b}) mice were between 6-8 weeks of age at the starting time of the experiments. All experiments were carried out within the ethical guidelines for the Karolinska Institute.

Induction of Collagen Induced Arthritis

Complete Freund's adjuvant (CFA) was prepared by mixing 100 mg of heat killed M. tuberculosis H37Ra (Difco, Detroit, Mich.) in 20 ml of incomplete Freund's adjuvant (IFA) (Difco). Chick CII (Sigma, St. Louis, Mo.) was solubilized at the concentration of 2 mg/ml, in 10 mM acetic acid (Sigma) by overnight incubation at 4° C. Chick CII was then emulsified 1:1 in CFA. Mice were injected intradermally in the base of the tail with 100 μl of emulsion.

NK Cell Depletion at Induction of Arthritis

Mice were injected intraperitoneally with mouse anti-NK1.1 (PK 136, BD Biosciences) depleting mAb (200 μg/mouse in PBS) one day prior to immunization with CII in CFA. The efficiency of depletion is monitored 2 days following NK1.1 injection, by FACS analysis of blood, stained with a pan-NK cell antibody (DX5, BD Biosciences) to verify the efficiency of depletion. A second round of depletion was performed 10 days after the first depletion (i.e. 9 days after immunization with CII). As controls, animals were injected. intraperitoneally with 200 μg/mouse of mouse IgG (Sigma) or with the same volume (200 μl) of PBS, in parallel with the NK1.1.

Clinical Assessment of Arthritis

Clinical scoring was performed using a visual scale were 1 equals redness and swelling in one joint (typically a toe), score 2 equals redness and swelling in more than one joint and a score of 3 is attributed when the entire paw is affected. Each animal can be given a maximum score of 12.

Incidence of CIA

FIG. 16 shows the incidence of disease in rice treated with anti-NK1.1 antibodies (NK1.1), IgG control (IgG1) and PBS alone (CII/CFA). Since no booster collagen II injection was performed only a few mice in the control group (i.e. CII/CFA) established CIA (1 of 10 mice with CIA at day 28 which resolved at day 42). In contrast, 8 of 10 mice that have been injected with anti-NK1.1 antibody established CIA at day 42, while only 5 of 10 mice in the IgG control treated mice showed signs of disease at day 42.

Total Disease Score

FIG. 17 shows the total arthritic score of animal treated with anti-NK1.1 antibodies (NK1.1), IgG control (IgG1) and PBS alone (CII/CFA). Mice treated with anti-NK1.1 antibodies display severe CIA in contrast to IgG-treated and PBS-treated control mice.

These foregoing results indicate that the presence of cells expressing the NK1.1 marker are necessary to protect from induction of CIA.

Peptide Treatment to Modulate Arthritis

The Qa1b binding protective qdm-peptide (AMAPRTLLL) (SEC ID NO: 115), and a nonamer (position 10-18; QMRPVSRAL) (SEC ID NO: 116) hsp60 signal-peptide derived from mouse hsp60 (Accession ID: P19226) and a synthetic qdmR5V-peptide (AMAPVTLLL) (SEC ID NO: 117) will be administered to mice before and after injection of collagen type II. These experiments will clarify a potential regulatory role of Qa1b and CD94/NKG2A interaction in the modulation of collagen-induced arthritis in this model.

Experimental Therapeutic Approach Using Qa-1b Binding Peptides

Like HLA-E, Qa-1b binds predominantly nonamer peptides derived from MHC class Icsignal peptides. This Qa-1b/peptide complex forms a functional ligand for CD94-NKG2A inhibitory receptors. There is evidence that HLA-E presents a nonameric peptide from the heat-shock protein 60 (hsp60) signal peptide during cellular distress. This presentation seem to be independent of transporter associated with antigen presentation 1 and 2 (TAP 1/2), which otherwise is necessary for loading of MHC class I signal peptides onto nascent HLA-E/Qa-1b molecules. Notably, HLA-E/hsp60 signal peptide complexes are not recognized by the CD94-NKG2A inhibitory receptor-pairs that recognize HLA-E complexed with proper MHC class I-signal peptides (Braud et al., Nature 391:795-799, 1991, incorporated herein by reference). Hsp60 is known to be highly expressed in arthritic tissues, both in human and in experimental arthritis models (Kleinau et al., Scand. J. Immunol. 33:195, 1991; Karlsson-Parra et al., Scand. J. Immunol. 31:283, 1991; Boog et al., J. Exp. Med. 175:1805, 1992, each incorporated herein by reference). Potentially, inflammatory foci contain predominantly HLA-E/hsp60 signal peptide complexes, which could be one important triggering factor for local NK cells. As a first step in the model of experimental arthritis (i.e. CIA) the potential therapeutic effect of administered Qa-1b binding MHC class I signal peptides (i.e. AMAPRTLLL) (SEC ID NO: 115) which is known to form relatively stable Qa-1b/peptide complex that can be recognized by the inhibitory CD94-NKG2A receptor pair will be evaluated. Other mice will receive irrelevant control peptides, and yet another group will receive nonameric hsp60 peptides. These peptides will initially be administered during established CIA to evaluate the therapeutic potential. Such peptides will also be administered prior to the injection of collagen II. Clinical and histological assessment of arthritis will be followed. Based on the results of peptide-therapy in experimental models of arthritis, these findings will be translated into human clinical trials to develop a novel therapeutic strategy that specifically target HLA-E and its capacity to form a functional ligand for human CD94-NKG2 receptor pairs.

Restoring HLA-E molecules with proper protective peptides that are recognized by CD94/NKG2A receptors will be useful in therapeutic regulation of ongoing chronic immune responses. In light of the findings that NK cells bearing predominantly CD94/NKG2A receptors are accumulated in the inflamed synovial fluid of arthritic patients, it should now be possible to therapeutically administer proper HLA-E binding peptides according to the methods of the invention to restore sufficient CD94/NKG2A mediated responses in the inflamed joint. Moreover, local administration of HLA-E binding peptides that uncouple CD94/NKG2A binding is believed to be of therapeutic value during cancer treatment to enhance NK cell (and T cell) mediated anti-tumor responses. Thus, HLA-E binding peptides are provided that constitute a switch whereby NK-cell mediated recognition of widely expressed HLA-E ligands is turned either on or off.

Although the foregoing invention has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation.

What is claimed is:

1-12. (canceled)

13. A treatment method, comprising
administering a peptide, wherein said peptide has a methionine at position 2, a proline at position 4, and a leucine or isoleucine at position 9, and wherein said peptide is capable of binding to human leukocyte antigen E (HLA-E) to form a peptide/HLA-E complex.

14. The method of claim 13, wherein said administering comprises administering a peptide having at least 65% amino acid sequence identity to an amino acid sequence of a peptide from a heat shock protein.

15. The method of claim 14, wherein said peptide from a heat shock protein is a signal sequence from a heat shock protein.

16. The method of claim 13, wherein said administering comprises administering a peptide having at least 65% amino acid sequence identity to an amino acid sequence of a peptide from a signal sequence of a human leukocyte antigen A, B, C, or G.

17. The method of claim 13, wherein said administering comprises administering a peptide having nine amino acid residues, wherein position 1 is V,A, L or I; position 3 is A, V, L, or I; position 5 is R; position 6 is S or T; and position 7 and 8 are independently selected from is A, I, L, F or M.

18. The method of claim 13, wherein said administering comprises administering a peptide having nine amino acid residues, wherein position 1, position 5, and position 7 are independently selected from A, L, I, M, or V; position 3 is selected from V, L, I, or A; position 6 is selected from S or T; and position 8 is selected from L or I.

19. The method of claim 13, wherein said administering comprises a peptide having nine amino acid residues, wherein position 1 is Q or N; position 3, position 5, and position 7 are independently selected from K, H, or R; position 6 is S or T; position 8 is A, L, I, M, or V.

20. The method of claim 13, wherein said treatment is for a disease or condition amenable to treatment by modulating an immune response.

21. The method of claim 20, wherein said disease or condition is an autoimmune disease.

22. The method of claim 21, wherein said autoimmune disease is multiple sclerosis or rheumatoid arthritis.

23. The method of claim 20, wherein said disease or condition is Crohn's disease, ulcerative colitis, transplant rejection, systemic lupus erythematosus, acute myeloid leukemia, ovarian cancer, atherosclerosis and insulin-dependent diabetes mellitus.

24. The method of claim 20, wherein said disease is a viral disease.

25. The method of claim 13, wherein said administering comprises administering said peptide entrapped in a liposome.

26. The method of claim 13, wherein said complex interacts with a CD94/NKG2 receptor present on a natural killer cell or a T cell.

27. The method of claim 26, wherein said CD94/NKG2 cellular receptor is a CD94/NKG2A cellular receptor.

28. The method of 26, wherein said complex interacts with a CD94/NKG2 receptor to cause a protective immune response.

29. The method of claim 13, further comprising administering a second therapeutic agent.

30. The method of claim 29, wherein said second therapeutic agent is an anti-viral agent, an anti-inflammatory agent, an anti-cancer agent or an immunosuppressant.

31. The method of claim 13, wherein said peptide is administered with a biocompatible polymer carrier.

32. The method of claim 31, wherein said carrier is a biodegradable polymer.

33. The method of claim 31, wherein said polymer is an absorption-promoting polymer.
